SEQUENCE LISTING (22 sequences)

SEQ ID NO: 1 - protein, 11 aa, synthetic construct
  REGION 1..11: note = Synthetic polymer
DLSKEPSISRE

SEQ ID NO: 2 - protein, 7 aa, synthetic construct
  REGION 1..7: note = Synthetic polymer
SKEPSIS

SEQ ID NO: 3 - protein, 8 aa, synthetic construct
  REGION 1..8: note = Synthetic polymer
DYKDDDDK

SEQ ID NO: 4 - DNA, 20 bp, synthetic construct
  misc_feature 1..20: note = Synthetic polymer
  source 1..20: mol_type = other DNA
ttatccatcctctcgctgct

SEQ ID NO: 5 - DNA, 19 bp, synthetic construct
  misc_feature 1..19: note = Synthetic polymer
  source 1..19: mol_type = other DNA
aggtgctgcttccctgact

SEQ ID NO: 6 - DNA, 20 bp, synthetic construct
  misc_feature 1..20: note = Synthetic polymer
  source 1..20: mol_type = other DNA
atggatgttggcttgtcctc

SEQ ID NO: 7 - DNA, 20 bp, synthetic construct
  misc_feature 1..20: note = Synthetic polymer
  source 1..20: mol_type = other DNA
taagcagttgtagcagacca

SEQ ID NO: 8 - DNA, 20 bp, synthetic construct
  misc_feature 1..20: note = Synthetic polymer
  source 1..20: mol_type = other DNA
gacatggcaggagtgtttga

SEQ ID NO: 9 - DNA, 20 bp, synthetic construct
  misc_feature 1..20: note = Synthetic polymer
  source 1..20: mol_type = other DNA
tttccatagccccctttacc

SEQ ID NO: 10 - DNA, 20 bp, synthetic construct
  misc_feature 1..20: note = Synthetic polymer
  source 1..20: mol_type = other DNA
tctatggtgcggagattgtg

SEQ ID NO: 11 - DNA, 20 bp, synthetic construct
  misc_feature 1..20: note = Synthetic polymer
  source 1..20: mol_type = other DNA
cttgatgtgcccgtccttgt

SEQ ID NO: 12 - DNA, 20 bp, synthetic construct
  misc_feature 1..20: note = Synthetic polymer
  source 1..20: mol_type = other DNA
cggcctcaactatatcgcca

SEQ ID NO: 13 - DNA, 20 bp, synthetic construct
  misc_feature 1..20: note = Synthetic polymer
  source 1..20: mol_type = other DNA
gcgcttcactctttcacgac

SEQ ID NO: 14 - DNA, 23 bp, synthetic construct
  misc_feature 1..23: note = Synthetic polymer
  source 1..23: mol_type = other DNA
cttgtctacctctaccccgacat

SEQ ID NO: 15 - DNA, 21 bp, synthetic construct
  misc_feature 1..21: note = Synthetic polymer
  source 1..21: mol_type = other DNA
gatccatgtcaaacgtgagcg

SEQ ID NO: 16 - DNA, 20 bp, synthetic construct
  misc_feature 1..20: note = Synthetic polymer
  source 1..20: mol_type = other DNA
atcgcaaacaagctgacctg

SEQ ID NO: 17 - DNA, 20 bp, synthetic construct
  misc_feature 1..20: note = Synthetic polymer
  source 1..20: mol_type = other DNA
agatccaggtttgaggtggg

SEQ ID NO: 18 - DNA, 21 bp, synthetic construct
  misc_feature 1..21: note = Synthetic polymer
  source 1..21: mol_type = other DNA
aagccgtcatcgtgaagaaca

SEQ ID NO: 19 - DNA, 21 bp, synthetic construct
  misc_feature 1..21: note = Synthetic polymer
  source 1..21: mol_type = other DNA
cttgatcttggatcgcttggc

SEQ ID NO: 20 - protein, 6 aa, synthetic construct
  REGION 1..6: note = Synthetic polymer
DLSKEP

SEQ ID NO: 21 - protein, 5 aa, synthetic construct
  REGION 1..5: note = Synthetic polymer
SISRE

SEQ ID NO: 22 - DNA, 74 bp, synthetic construct
  misc_feature 1..74: note = Synthetic polymer
  source 1..74: mol_type = other DNA
gttctgagaaaagtagttctgagaaaagtagttctgagaaaagtagttctgagaaaagtagttctgagaaaagt

(19) United States
(12) Patent Application Publication
(10) Pub. No.: US 2025/0177473 A1
(43) Pub. Date: Jun. 5, 2025

(54) PEPTIDE REGULATORS OF METABOLISM

(71) Applicant: Metabico, Inc., Newton, MA (US)

(72) Inventors: Nikita MITKIN, Newton, MA (US); Vsevolod PAVSHINTSEV, Newton, MA (US); Anton MALYSHEV, Newton, MA (US); Igor DORONIN, Newton, MA (US); Gennady BABKIN, Newton, MA (US); Marianna SADAGURSKI, Newton, MA (US)

(21) Appl. No.: 18/838,784

(22) PCT Filed: Feb. 16, 2023
(86) PCT No.: PCT/US2023/062738

§ 371 Date: Aug. 15, 2024

Related U.S. Application Data
(60) Provisional application No. 63/311,303, filed on Feb. 17, 2022.

(51) Int. Cl. A61K 38/01; A61K 31/198; A61K 31/245; A61K 31/575; A61K 31/69; A61K 31/7076; A61K 38/06; A61K 38/12; A61K 38/47; A61K 38/57; A61P 3/10

(52) U.S. Cl. CPC A61K 38/018; A61K 31/198; A61K 31/245; A61K 31/575; A61K 31/69; A61K 31/7076; A61K 38/06; A61K 38/12; A61K 38/47; A61K 38/57; A61P 3/10

(57) ABSTRACT

The disclosure relates to synthetic peptides that are capable of modulating metabolism and find uses e.g. in treating metabolic diseases or disorders.

### CROSS-REFERENCE TO RELATED APPLICATIONS

This application claims the benefit of U.S. Provisional Patent Application No. 63/311,303, filed Feb. 17, 2022, the entire contents of which are hereby incorporated by reference in their entirety.

### FIELD

The present disclosure relates to compositions that include peptide therapeutic agents, to treat metabolic disorders such as type II diabetes and obesity.

### DESCRIPTION OF THE TEXT FILE SUBMITTED ELECTRONICALLY

This application contains a Sequence Listing in XML format submitted electronically herewith via Patent Center. The contents of the XML copy, created on Feb. 15, 2023, is named “MBO-001PC_sequence_listing.xml” and is 28,543 bytes in size. The Sequence Listing is incorporated herein by reference in its entirety.

### BACKGROUND

Metabolic disorders are a group of heterogeneous conditions with different epidemiology, causes, and clinical manifestations. Metabolic disorders affect all age groups and are of great social and clinical importance, as they lead to large financial costs for health systems all over the world. Among the most prevalent metabolic disorders, obesity and type 2 diabetes (T2D) affect hundreds of millions of individuals worldwide, and the numbers are being predicted to rise for the next few decades. Although various drugs for the treatment of metabolic disorders have been developed, a substantial medical need for novel, effective treatments for the vast majority of metabolic conditions still persists. For instance, sodium-glucose transport protein 2 (SGLT2) inhibitors, considered as the latest generation medications for T2D treatment, provide efficient blood glucose levels control and alleviation of insulin resistance state by inhibiting filtered glucose reabsorption in the kidneys. However, they have a wide range of side effects, including urinary tract infection and episodes of ketoacidosis (Hsia (2017). An update on SGLT2 inhibitors for the treatment of diabetes mellitus. Current opinion in endocrinology, diabetes, and obesity, 24(1), 73). SGLT2 inhibitors are contraindicated for patients with renal insufficiency (Scheen, A. J. (2015). Pharmacokinetics, pharmacodynamics, and clinical use of SGLT2 inhibitors in patients with type 2 diabetes mellitus and chronic kidney disease. Clinical pharmacokinetics, 54(7), 691-708), which is exceedingly frequently occurred in T2D patients (Thomas (2016). Changing epidemiology of type 2 diabetes mellitus and associated chronic kidney disease. Nature Reviews Nephrology, 12(2), 73-81). Another group of next-generation medications for T2D treatment, glucagon-like peptide-1 (GLP-1) receptor agonists, have severe gastrointestinal adverse effects (nausea, vomiting, and diarrhea) in 10-30% of patients leading to therapy discontinuation (Trujillo et al. (2021). GLP-1 receptor agonists: an updated review of head-to-head clinical studies. Therapeutic Advances in Endocrinology and Metabolism, 12, 2042018821997320). Also, inadequate blood glucose control and inability to induce weight loss in patients are reported for these medications as the frequent reasons for discontinuation (Sikirica et al. (2017). Reasons for discontinuation of GLP1 receptor agonists: datafrom a real-world cross-sectional survey of physicians and their patients with type 2 diabetes. Diabetes, metabolic syndrome, and obesity: targets and therapy, 10, 403).

As for obesity treatment, next-generation therapeutics are focused on mimicking central pathways regulating appetite and food intake. GLP1 receptor agonists are considered the most common novel anti-obesity drugs, but they share the same side effects problems as in the case of T2D treatment. Also, their effects last only when the drug is onboard, lacking long-term positive changes when it is no longer administered. Special attention is paid to mechanisms that affect appetite in patients carrying genetic pathologies associated with pro-opiomelanocortin or leptin receptor deficiency or in individuals with leptin resistance. In this area, a range of peptide-based agents (e.g., without limitation, agonists of MC4 melanocortin receptors) are being tested, but their efficiency remains low. According to a recent clinical trial, only 35% of patients achieved a 10% bodyweight reduction after a year of treatment (Haws et al. (2021). The efficacy and safety of setmelanotide in individuals with Bardet-Biedl syndrome or Alström syndrome: Phase 3 trial design. Contemporary Clinical Trials Communications, 22, 100780). Concerning SGLT2 inhibitors which are considered as a promising medication for T2D treatment, they demonstrate no significant efficiency in reduction of appetite, body- or fat-mass in overweighted non-diabetic subjects (Ryan (2020). Sodium Glucose Co-Transporter 2 Inhibition Does Not Favorably Modify the Physiological Responses to Dietary Counselling in Diabetes-Free, Sedentary Overweight, and Obese Adult Humans. Nutrients, 12(2), 510.)

Prediabetes (intermediate hyperglycemia) is an intermediate metabolic state with glycemic parameters above normal but below diabetic levels. Prediabetes is a condition that could be modulated with medications, lowering the risk of the development of diabetes. Currently, only metformin is prescribed as an off-label drug for patients with prediabetes in the US. Although there are no medications for prediabetes treatment approved by the US Food and Drug Administration (FDA), the American Diabetes Association (ADA) recommends considering metformin for patients with prediabetes and high body mass index (BMI). Metformin controls blood glucose levels and significantly improves cardiovascular outcomes in both prediabetic and diabetic individuals. On the other hand, metformin treatment lacks long-term effects on weight loss, making it ineffective in obesity treatment. It has substantial gastrointestinal side effects in a large number of cases that lead to the drug discontinuation. Additionally, according to current FDA guidelines, metformin is contraindicated in renal impairment patients (Inzucchi (2014). Metformin in patients with type 2 diabetes and kidney disease: a systematic review. Jama, 312(24), 2668-2675.), that limits its use in diabetic or prediabetic subjects due to high association of these states with kidney impairments (Kim (2019) Association between prediabetes (defined by HbA1C, fasting plasma glucose, and impaired glucose tolerance) and the development of chronic kidney disease: a 9-year prospective cohort study. BMC nephrology, 20(1), 1-6).

There remains a great demand for effective and safe therapeutics for the treatment of a wide range of metabolic disorders with novel mechanisms of action.

### SUMMARY

In aspects, the present disclosure provides compositions and methods that are useful for the treatment of various metabolic disorders including diabetes, lysosomal storage diseases, hypercholesterolemia, obesity, as well as inherited metabolic disorders.

In aspects, the present disclosure provides a composition comprising a synthetic peptide, the synthetic peptide comprising or consisting of an amino acid sequence derivable or derived from one or more milk hydrolysate proteins, wherein the synthetic peptide is capable of modulating metabolism.

In embodiments, the synthetic peptide comprises or consists of about 4 to about 12 amino acids.

In aspects, a biologically active peptide, in the form of a pharmaceutical composition is used for the treatment of diabetes, obesity, and other metabolic disorders.

In embodiments, a composition is provided that comprises a synthetic peptide, that is defined by the general formula 1: X₁X₂X₃X₄R₁R₂R₃R₄Y₁Y₂Y₃ (I).

In embodiments, X₁ is absent or a polar and negatively charged hydrophilic amino acid, optionally selected from aspartate (D) and glutamate (E);

X₂ is absent or a hydrophobic, aliphatic amino acid, optionally selected from glycine (G), alanine (A), leucine (L), isoleucine (I), methionine (M), and valine (V);

X₃ is absent or a polar and neutral charged hydrophilic amino acid, optionally selected from asparagine (N), glutamine (Q), serine (S), threonine (T), proline (P), and cysteine (C);

X₄ is absent or a polar and positively charged hydrophilic amino acid, optionally selected from arginine (R) and lysine (K);

R1 is selected from a polar and negatively charged hydrophilic amino acid, optionally selected from aspartate (D) and glutamate (E);

R₂ is selected from a polar and neutral charged hydrophilic amino acid, optionally selected from asparagine (N), glutamine (Q), serine (S), threonine (T), proline (P), and cysteine (C);

R₃ is selected from a polar and neutral charged hydrophilic amino acid, optionally selected from asparagine (N), glutamine (Q), serine (S), threonine (T), proline (P), and cysteine (C);

R₄ is selected from a hydrophobic, aliphatic amino acid, optionally selected from glycine (G), alanine (A), leucine (L), isoleucine (I), methionine (M), and valine (V);

Y₁ is absent or a polar and neutral charged hydrophilic amino acid, optionally selected from asparagine (N), glutamine (Q), serine (S), threonine (T), proline (P), and cysteine (C);

Y₂ is absent or a polar and positively charged hydrophilic amino acid, optionally selected from arginine (R) or lysine (K); and

Y₃ is absent a polar and negatively charged hydrophilic amino acid, optionally selected from aspartate (D) and glutamate (E).

In embodiments, X₁ is aspartate (D).

In embodiments, X₂ is leucine (L).

In embodiments, X₃ is serine (S).

In embodiments, X₄ is lysine (K).

In embodiments, R₁ is glutamate (E).

In embodiments, R₂ is proline (P).

In embodiments, R₃ is serine (S).

In embodiments, R₄ is isoleucine (I).

In embodiments, Y₁ is serine (S).

In embodiments, Y₂ is arginine (R).

In embodiments, Y₃ is glutamate (E).

In embodiments, R₁ is glutamate (E); R₂ is proline (P); R₃ is serine (S); and R₄ is isoleucine (I).

In embodiments, X₃ is serine (S); X₄ is lysine (K); R₁ is glutamate (E); R₂ is proline (P); R₃ is serine (S); R₄ is isoleucine (I); and Y₁ is serine (S).

In embodiments, X₁ is aspartate (D); X₂ is leucine (L); X₃ is serine (S); X₄ is lysine (K); R₁ is glutamate (E); R₂ is proline (P); R₃ is serine (S); R₄ is isoleucine (I); Y₁ is serine (S); Y₂ is arginine (R); and Y₃ is glutamate (E).

In embodiments, the regulatory peptides and their analogs described herein act as positive allosteric modulators of neuropeptide S receptor (NPSR1), antagonists of FFAR2 (GPR43), and GPR109A receptors, positive allosteric modulators of LPAR3 receptor, and/or inverse agonists of M2 muscarinic receptor. In embodiments the action of the peptides of the present disclosure through the described targets leads to induction of stimulatory G protein α-subunit (Gsa)-cAMP axis in different tissues resulted in activation of intracellular Stat3 signaling. Given the link between Gsa-cAMP-Stat3 signaling and mechanisms of appetite regulation, glucose homeostasis, insulin resistance, and fat mass decrease, the present peptides are effective at preventing or treating various metabolic disorders, including type 2 diabetes and obesity. Non-limiting examples of conditions that are treated using the described regulatory peptide include, embodiments, diabetes mellitus (DM) (e.g., without limitation, Type 1 diabetes, Type 2 diabetes, hybrid form of diabetes (immune-mediated diabetes of adults, ketosis-prone type 2 diabetes), hyperglycemia first detected during pregnancy (DM in pregnancy, gestational DM)), intermediate hyperglycemia (e.g., without limitation, impaired fasting glucose, impaired glucose tolerance, other specified intermediate hyperglycemia or unspecified intermediate hyperglycemia), another insulin-resistance syndrome, other specified or unspecified disorders of glucose regulation and pancreatic internal secretion, overweight (e.g., without limitation, overweight in infants, children or adolescents, overweight in adults, localised adiposity), obesity (e.g., without limitation, obesity due to energy imbalance including but not limited by obesity in children or adolescents and obesity in adults, drug-induced obesity, obesity hypoventilation syndrome, Prader-Willi syndrome, other specified obesity, unspecified obesity), feeding or eating disorders (e.g., without limitation, bulimia nervosa, binge eating disorder, other specified feeding or eating disorders), non-alcoholic fatty liver disease (e.g., without limitation, non-alcoholic fatty liver disease without non-alcoholic steatohepatitis, non-alcoholic steatohepatitis), hyperlipoproteinaemia (e.g., without limitation, hypercholesterolaemia, hypertriglyceridaemia, mixed hyperlipidaemia, other specified hyperlipoproteinaemia), inborn errors of metabolism (e.g., without limitation, inborn errors of carbohydrate metabolism, inborn errors of lipid metabolism, inborn errors of energy metabolism). In embodiments, diabetes is other specific types of diabetes: monogenic diabetes, disease of the exocrine pancreas, endocrine disorders, drug- or chemical-induced diabetes, infection-related diabetes, uncommon specific forms of immune-mediated diabetes, and other genetic syndromes sometimes associated with diabetes. In embodiments, other specified obesity includes but is not limited by obesity due to melanocortin-4 receptor deficiency and leptin-related genetic obesity.

In embodiments, the peptides are optionally chemically modified. In embodiments, the chemical modification is selected from amidation, methylation, and acetylation of one or more of the amino acids. Additional chemical modifications can include the addition of formyl, pyroglutamyl (pGlu), one or more fatty acids, urea, carbamate, sulfonamide, alkylamine, or any combination thereof. Additional chemical modifications include incorporating non-natural amino acids into certain positions in the peptide. Non-limiting examples of the non-natural amino acids include D-amino acids, N-methylated (or N-alkylated) amino acids, alpha-substituted alpha-amino acids, beta-substituted alpha-amino acids, beta-amino acids, and gamma-amino acids.

In embodiments, the composition includes a pharmaceutically acceptable carrier. In embodiments, the composition can further include a delivery vehicle which is, e.g., without limitation, a liposome, a nanoparticle, or a polysaccharide. In embodiments, the composition is administered to a subject determined to be in need of treatment via various routes, and in aspects, the composition is formulated for intranasal administration, oral administration, or subcutaneous administration.

### BRIEF DESCRIPTION OF THE FIGURES

FIG. 1 illustrates the influence of the selected peptides from bovine milk hydrolysate on mRNA expression levels of TSC1, TSC2, pS6K1, pAKT, IRS2, SREBP-1, and Stat3 genes in the primary mouse fibroblasts cell culture. The expression levels of the studied genes were estimated by real-time PCR. Expression levels were normalized to values for the housekeeping gene RPL27. The results are presented as the mean±standard error of the mean (SEM) for 3 biological replicates. *−p<0.05 in respect to Control. One-way ANOVA with Fisher's LSD post hoc test.

FIG. 2 illustrates the effect of different doses of the DLSKEPSISRE (SEQ ID NO: 1) peptide on the level of IRS2 gene mRNA expression in primary mouse fibroblasts. The expression levels of IRS2 gene were estimated by real-time PCR. Expression levels were normalized to the housekeeping gene RPL27. The results are presented as the mean±SEM for 3 biological replicates. *−p<0.05 in respect to Control. One-way ANOVA with Fisher's LSD post hoc test.

FIG. 3 illustrates the activation of IRS2 gene expression in response to DLSKEPSISRE (SEQ ID NO: 1) peptide both under normal and stress conditions (inflammatory stress and high-glucose medium), the effect has the same direction as the action of metformin. The expression levels of IRS2 gene were estimated by real-time PCR. Expression levels were normalized to values for the housekeeping gene RPL27. The results are presented as the mean±SEM for 3 biological replicates. *−p<0.05 in respect to Control+LPS. One-way ANOVA with Fisher's LSD post hoc test.

FIG. 4 illustrates that DLSKEPSISRE (SEQ ID NO: 1) peptide performs a significant anti-inflammatory effect comparable to metformin. The expression levels of the studied genes were estimated by real-time PCR. Expression levels were normalized to values for the housekeeping gene RPL27. The results are presented as the mean±SEM for 3 biological replicates. *−p<0.05 in respect to Control+LPS. One-way ANOVA with Fisher's LSD post hoc test.

FIG. 5 illustrates that DLSKEPSISRE (SEQ ID NO: 1) peptide pharmacophores SKEPSIS (SEQ ID NO: 2) and EPSI induce IRS2 gene expression in mouse primary fibroblasts in the same manner as a full-size peptide. Expression levels were normalized to values for the housekeeping gene RPL27. The results are presented as the mean±SEM for 3 biological replicates. *−p<0.05 in respect to Control. One-way ANOVA with Fisher's LSD post hoc test.

FIG. 6 illustrates the influence of SKEPSIS (SEQ ID NO: 2) and EPSI peptides on expression levels of pro-inflammatory cytokines in primary mouse fibroblasts induced with LPS. Expression levels were normalized to values for the housekeeping gene RPL27. The results are presented as the mean±SEM for 3 biological replicates. *−p<0.05 in respect to Control+LPS. One-way ANOVA with Fisher's LSD post hoc test.

FIG. 7 illustrates the influence of SKEPSIS (SEQ ID NO: 2) and EPSI peptides on IRS2 expression in primary mouse fibroblasts induced with LPS. IRS2 expression levels were estimated by real-time PCR. Expression levels were normalized to values for the housekeeping gene RPL27. The results are presented as the mean±SEM for 3 biological replicates. *−p<0.05 in respect to Control+LPS. One-way ANOVA with Fisher's LSD post hoc test.

FIG. 8 illustrates the activation of M67 (Stat1/Stat3) luciferase reporter in HepG2 cells in response to incubation with EPSI peptide. The results are presented as the mean±SEM for 3 biological replicates. *− p<0.05 in respect to Control. One-way ANOVA with Fisher's LSD post hoc test.

FIG. 9 illustrates the absence of the effect of EPSI peptide on the activity of NFATc1-, AP1-, Stat5- or Stat4-luciferase reporters in HepG2 cells. The results are presented as the mean±SEM for 3 biological replicates. *−p<0.05 in respect to Control. One-way ANOVA with Fisher's LSD post hoc test.

FIG. 10 illustrates the ability of EPSI peptide to induce the activity of M67 (Stat1/Stat3), luciferase reporter, in HepG2 cells in the presence of inhibitors of main Stat3-inducing receptors. The results are presented as the mean±SEM for 3 biological replicates. *−p<0.05 in respect to Control. One-way ANOVA with Fisher's LSD post hoc test.

FIG. 11 illustrates the dose-dependent action of EPSI peptide as an antagonist of FFAR2 receptor in HEK-293 cell line constitutively expressing FFAR2. The results are represented as % of inhibition of intracellular Ca2+ levels evoked by control reference agonist (1 mM sodium acetate). The results are presented as the mean±SEM for 3 replicates. *−p<0.05 in respect to the cells activated with control reference agonist without peptide application. One-way ANOVA with Fisher's LSD post hoc test.

FIGS. 12A-12B illustrates the ability of EPSI peptide to activate pStat3 in the hypothalamus of experimental mice in 60 minutes after its intranasal administration detected by immunohistochemical staining of brain slices with anti-pStat3 (Y705) antibodies. FIG. 12A shows pStat3-positive cells in the hypothalamic sections of experimental mice. Fluorescence microscopy. Scale segment—100 μm. FIG. 12B shows % of pStat3-positive cells in hypothalamic sections of experimental animals. Represented as the mean±SD. *− p<0.05, **−p<0.01—statistical significance based on a single-factor ANOVA. test.

FIG. 13 illustrates the weight of the food consumed at 30-minute Intervals in the “Home cage feed consumption” test. Intact male CBA and C57Bl/6 hybrids mice after deprivation of food for 12 hours (overnight). EPSI was injected i.n. in 5 mg/kg dose, leptin was injected i.p. in 1 mg/kg dose before testing. Each bar represents an average weight of the food consumed at 30-minute Intervals per group±SEM. Significant difference from the Control group is denoted by the “*” symbol (repeated measures ANOVA a posteriori analysis by Fisher's test, the parameter “Group and interval”; p<0.05).

FIG. 14 illustrates the weight of the food consumed in 1.5 hours of observation in the “Home cage feed consumption” test. Intact male CBA and C57Bl/6 hybrids mice after deprivation of food for 12 hours (overnight). EPSI was injected i.n. in 5 mg/kg dose, leptin was injected i.p. in 1 mg/kg dose before testing. Each bar represents an average weight of the food consumed in 1.5 hours of observation per group±SEM. Significant difference from the Control group is denoted by the “*” symbol (one-way ANOVA followed by Fisher's LSD test; p<0.05).

FIG. 15. illustrates the concentration of blood glucose (mmol/L) after providing a 30% sucrose solution to Sprague-Dawley rats (High sucrose diet, HSD). Each bar represents an average total blood glucose concentration±SEM. The statistical significance was calculated by multivariate analysis of variance (two-way ANOVA) with a posteriori analysis by Fisher's test; *−p<0.05 for the “Group” parameter; #-p<0.05 according to the “Day” parameter.

FIG. 16. illustrates the dynamics of glucose concentration (mmol/L) in the blood in the Glucose tolerance test after EPSI administration on 0 h, 2 h, 12 h, 24 h. (i.p. 1 and 10 mg/kg) before the intragastric glucose administration. Sprague-Dawley male rats after 5 weeks of 30% glucose solution consumption (High sucrose diet, HSD). Results are presented as mean values for the group.

FIG. 17 illustrates the area under the curve (AUC, mmol/L/min) of changes in blood glucose concentration in the “Glucose Tolerance” test after EPSI administration on 0 h, 2 h, 12 h, 24 h. (i.p. 1 and 10 mg/kg) before the intragastric glucose administration. Sprague-Dawley male rats after 5 weeks of 30% glucose solution consumption (High sucrose diet, HSD). Each bar represents an average AUC per group±SEM. Significant difference from the Control HSD group is denoted by the “#” symbol (one-way ANOVA followed by Fisher's LSD test; p<0.05).

FIG. 18 illustrates low-density lipoproteins (LDL) concentration in blood serum, mmol/L. Sprague-Dawley male rats after 6 weeks of 30% glucose solution consumption (High sucrose diet, HSD). EPSI administration 2 h, 12 h. before testing (i.p. 1 and 10 mg/kg). Each bar represents an average LDL concentration per group±SEM. Significant difference from the Control group is denoted by the “*” symbol; significant difference from the Control HSD group is denoted by the “#” symbol (one-way ANOVA followed by Fisher's LSD test; p<0.05).

FIG. 19 illustrates glucose concentration in blood serum, mmol/L. Sprague-Dawley male rats after 6 weeks of 30% glucose solution consumption (High sucrose diet, HSD). EPSI administration 2 h, 12 h. before testing (i.p. 1 and 10 mg/kg). Each bar represents an average glucose concentration per group±SEM. Significant difference from the Control group is denoted by the “*” symbol; significant difference from the Control HSD group is denoted by the “#” symbol (one-way ANOVA followed by Fisher's LSD test; p<0.05).

FIG. 20 illustrates insulin concentration in blood serum, mIU/L. Sprague-Dawley male rats after 6 weeks of 30% glucose solution consumption (High sucrose diet, HSD). EPSI administration 2 h, 12 h. before testing (i.p. 1 and 10 mg/kg). Each bar represents an average insulin concentration per group±SEM. Significant difference from the Control group is denoted by the “*” symbol; significant difference from the Control HSD group is denoted by the “#” symbol (one-way ANOVA followed by Fisher's LSD test; p<0.05).

FIG. 21 illustrates the Insulin resistance index (HOMA-IR). Sprague-Dawley male rats after 6 weeks of 30% glucose solution consumption (High sucrose diet, HSD). EPSI administration 2 h, 12 h. before testing (i.p. 1 and 10 mg/kg). Each bar represents an average insulin resistance index per group±SEM. Significant difference from the Control group is denoted by the “*” symbol; significant difference from the Control HSD group is denoted by the “#” symbol (one-way ANOVA followed by Fisher's LSD test; p<0.05).

FIG. 22 illustrates levels of the p-Akt (Thr308-phosphorylated form) normalized to the GAPDH in the liver of experimental animals in response to insulin administration. Sprague-Dawley male rats after 6 weeks of 30% glucose solution consumption (High sucrose diet, HSD). EPSI administration 2 h, 12 h. before testing (i.p. 1 and 10 mg/kg). Each bar represents an average level of the p-Akt (Thr308-phosphorylated form) per group±SEM. Significant difference from the Control group is denoted by the “*” symbol; significant difference from the Control HSD group is denoted by the “#” symbol (one-way ANOVA followed by Fisher's LSD test; p<0.05).

FIG. 23 illustrates levels of the p-Akt (Ser473-phosphorylated form) normalized to the GAPDH in the liver of experimental animals in response to insulin administration. Sprague-Dawley male rats after 6 weeks of 30% glucose solution consumption (High sucrose diet, HSD). EPSI administration 2 h, 12 h. before testing (i.p. 1 and 10 mg/kg). Each bar represents an average level of the p-Akt (Ser473-phosphorylated form) per group±SEM. Significant difference from the Control group is denoted by the “*” symbol; significant difference from the Control HSD group is denoted by the “#” symbol (one-way ANOVA followed by Fisher's LSD test; p<0.05).

FIG. 24 illustrates levels of the P-p70 S6 Kinase (Thr421/Ser424) normalized to the GAPDH in the liver of experimental animals in response to insulin administration. Sprague-Dawley male rats after 6 weeks of 30% glucose solution consumption (High sucrose diet, HSD). EPSI administration 2 h, 12 h. before testing (i.p. 1 and 10 mg/kg). Each bar represents an average level of the P-p70 S6 Kinase (Thr421/Ser424) per group±SEM. Significant difference from the Control group is denoted by the “*” (one-way ANOVA followed by Fisher's LSD test; p<0.05).

FIG. 25 illustrates the dynamics of glucose concentration (mmol/L) in the blood in the Glucose tolerance test after EPSI and metformin acute administration 2 h. before the intragastric glucose administration. EPSI i.n. 5 mg/kg and i.p. 5 mg/kg; metformin p.o. 5 mg/kg). Adult C57Bl/6 mice, high-fat diet (45% fat) for 4 months. STD—control group on a standard diet; HFD—control group on a high-fat diet. Results are presented as mean values for the group.

FIG. 26 illustrates the blood glucose concentration (mmol/l) in the “Glucose tolerance” test 15 minutes after intragastric glucose administration. EPSI i.n. 5 mg/kg and i.p. 5 mg/kg; metformin p.o. 5 mg/kg acute administration 2 h. before the test. Adult C57Bl/6 mice, high-fat diet (45% fat) for 4 months). Each bar represents an average level of the blood glucose concentration per group±SEM. Significant difference from the STD Control group is denoted by the “*” symbol; significant difference from the HFD control group is denoted by the “#” symbol (one-way ANOVA followed by Fisher's LSD test; p<0.05).

FIG. 27 illustrates the average body weight (gram) of adult C57Bl/6 mice after a high-fat diet (45% fat) for 4 months before the introduction of substances and after 4 weeks of daily single administration. EPSI i.n. 5 mg/kg and i.p. 5 mg/kg; metformin p.o. 5 mg/kg. Each bar represents an average body weight per group±SEM. The significant difference in experimental groups before and after drug administration is denoted by the “*” symbol (repeated-measures ANOVA followed by Fisher's LSD test; p<0.05).

FIG. 28 illustrates the dynamics of overweight changes in animals after a high-fat diet (45% fat) for 4 months. The values for the experimental groups are normalized to the “HFD Control” group values. Daily single administration (4 weeks) started after 122 days of the experiment. EPSI i.n. 5 mg/kg and i.p. 5 mg/kg; metformin p.o. 5 mg/kg.

FIG. 29 illustrates food consumption by animals (kcal/kg/day), average values per group, 4th week of the introduction of substances. Each bar represents an average level of food consumption per group±SEM. Significant difference from the STD Control group is denoted by the “*” symbol; significant difference from the HFD control group is denoted by the “*” symbol (one-way ANOVA followed by Fisher's LSD test; p<0.05).

FIG. 30 illustrates blood glucose concentration in experimental groups (mmol/l), 4 weeks of substance administration. Each bar represents an average level of the glucose concentration per group±SEM. Significant difference from the STD Control group is denoted by the “*” symbol; significant difference from the HFD control group is denoted by the “#” symbol (one-way ANOVA followed by Fisher's LSD test; p<0.05).

FIG. 31 illustrates insulin resistance index (HOMA-IR), 4 weeks of substance administration. Each bar represents an average level of the HOMA-IR per group±SEM. Significant difference from the STD Control group is denoted by the “*” symbol; significant difference from the HFD control group is denoted by the “#” symbol (one-way ANOVA followed by Fisher's LSD test; p<0.05).

FIG. 32 illustrates the concentration of TNF in blood serum (ng/ml), 4 weeks of substance administration. Each bar represents an average level of the TNF concentration per group±SEM. Significant difference from the STD Control group is denoted by the “*” symbol; significant difference from the HFD control group is denoted by the “#” symbol (one-way ANOVA followed by Fisher's LSD test; p<0.05).

FIG. 33. illustrates visceral fat weight (surrounding the epididymis), 4 weeks of substance administration. Each bar represents an average visceral fat weight per group±SEM. Significant difference from the STD Control group is denoted by the “*” symbol; significant difference from the HFD control group is denoted by the “#” symbol (one-way ANOVA followed by Fisher's LSD test; p<0.05).

FIG. 34 illustrates the linear size of visceral fat adipocytes (μm). 4 weeks of substance administration. Each bar represents an average linear size of visceral fat adipocytes per group±SEM. Significant difference from the STD Control group is denoted by the “*” symbol; significant difference from the HFD control group is denoted by the “#” symbol (one-way ANOVA followed by Fisher's LSD test; p<0.05).

### DETAILED DESCRIPTION

Peptide compositions are provided herein, which have use in, e.g., without limitation, treatment of diabetes, obesity, and other metabolic disorders. In aspects, peptide-based therapeutical compositions for a range of metabolic disorders are provided. The peptides have shown a prominent regulation of expression of genes involved in the pathogenesis of various metabolic conditions, which proposes high efficacy of peptide compositions. The peptides act, inter alia, as potent positive allosteric modulators of neuropeptide S receptor (NPSR1) which is a target of the described peptides. Additionally, inter alia, the peptides act as antagonists of FFAR2 (GPR43) and GPR109A receptors, positive allosteric modulators of LPAR3 receptor, and inverse agonists of M2 muscarinic receptor. The peptides induce intracellular Stat3 signaling in different tissues (e.g., without limitation, the brain, in particular, the hypothalamus) applying the described targets. The compositions in accordance with the present disclosure provide safe and effective treatment.

In embodiments, the present peptides of the described group act as potent positive allosteric modulators of NPSR1 (neuropeptide S receptor). Neuropeptide S is considered as a potent regulator of appetite and eating behavior (Botticelli (2021). The Neural Network of Neuropeptide S (NPS): Implications in food intake and gastrointestinal functions. Pharmaceuticals, 14(4), 293) and a stimulator of fatty acids oxidation in adipose tissue. Single nucleotide polymorphism in NPSR1 gene locus is associated with obesity, and serum levels of neuropeptide S are significantly decreased in obese individuals (Ahmad (2020). Neuropeptide S receptor gene Asn107 polymorphism in obese male individuals in Pakistan. PloS one, 15(12), e0243205). NPSR1 is a G-protein-coupled receptor that acts through both Gaq and Gas. It is highly expressed in the hypothalamus and can induce Stat3 signaling through Gas activation that could potentially serve as a pathway mimicking leptin action (Cline (2007). Anorexigenic effects of central neuropeptide S involve the hypothalamus in chicks (Gallus gallus). Comparative Biochemistry and Physiology Part A: Molecular & Integrative Physiology, 148(3), 657-663). In hypothalamus neuropeptide, S is known to reduce the activity of c-Fos transcription factor (Cline (2007). Anorexigenic effects of central neuropeptide S involve the hypothalamus in chicks (Gallus gallus). Comparative Biochemistry and Physiology Part A: Molecular & Integrative Physiology, 148(3), 657-663) that is typical for leptin action and could play the key role in the inhibition of ghrelin and orexin production, the main regulators of eating behavior (Ge (2020). Role of leptin in the regulation of food intake in fasted mice. Journal of cellular and molecular medicine, 24(8), 4524-4532). Additionally, the shows the ability of regulatory peptides of the described group to act as antagonists of FFAR2 (GPR43) and GPR109A receptors. Mechanistically, inhibition of both GPR109A and FFAR2 should be associated with reduced Gai signaling that results in induction of Gas signaling and elevated cAMP levels in different tissues (Priyadarshini (2018). Role of short-chain fatty acid receptors in intestinal physiology and pathophysiology. Comprehensive Physiology, 8(3), 109) typical for the action of agonists of metabolically relevant receptors considered as therapeutic targets (e.g., without limitation, GLP1R, GIP-R, AMY2, MC4R). Also, as described above, Gas-cAMP induction is typical for NPSR1 activation, therefore the action of the peptides as FFAR2/GPR109A antagonists and activators of NPSR1 should be synergistic. FFAR2 inhibition is considered a promising strategy of type 2 diabetes treatment (Tang (2015). Loss of FFA2 and FFA3 increases insulin secretion and improves glucose tolerance in type 2 diabetes. Nature medicine, 21(2), 173-177): FFAR2 is highly expressed in the pancreas, and its activation is associated with reduced insulin secretion that in the long-term leads to insulin resistance progression (Priyadarshini (2015). An acetate-specific GPCR, FFAR2, regulates insulin secretion. Molecular endocrinology, 29(7), 1055-1066). Inhibition of GPR109A, a protein that is primarily expressed in adipocytes and hepatocytes, results in alleviation of insulin resistance conditions (Heemskerk (2014). Long-term niacin treatment induces insulin resistance and adrenergic responsiveness in adipocytes by adaptive downregulation of phosphodiesterase 3B. American Journal of Physiology-Endocrinology and Metabolism, 306(7), E808-E813) and induction of lipolysis (Geisler (2021). The Role of GPR109a Signaling in Niacin Induced Effects on Fed and Fasted Hepatic Metabolism. International journal of molecular sciences, 22(8), 4001).

In embodiments, the present peptides induce Stat3 activity (increase the level of tyrosine 705 phosphorylation) in the brain, in particular in the hypothalamus. Probably, this type of action could apply the same pathways as in case of leptin, a hormone produced and released from adipose tissue, a key regulator of appetite, thermogenesis, and blood glucose. Activation of leptin receptor (LEPR) in hypothalamic cells is associated with induction of JAK-STAT signaling pathway which is involved in the regulation of eating behavior (Kwon (2016). Leptin signaling pathways in hypothalamic neurons. Cellular and Molecular Life Sciences, 73(7), 1457-1477). The level of phosphorylated Stat3 is believed to be the main indicator of both LEPR activation and induction of the LEPR-independent pathways responsible for appetite regulation (Ladyman (2013). JAK-STAT and feeding. Jak-stat, 2(2), e23675). Activation of pStat3 in the same hypothalamic neurons as it is observed in the case of leptin could be potentially associated with the ability of the described regulatory peptides to induce production of polypeptide hormone pro-opiomelanocortin (POMC) by POMC neurons with its further processing to mature α-melanocyte-stimulating hormone α-MSH (Baldini (2019). The melanocortin pathway and control of appetite-progress and therapeutic implications. Journal of Endocrinology, 241(1), R1-R33). α-MSH binding on melanocortin receptor 4 (MC4R) expressed on neurons of the paraventricular nucleus (PVN) of the hypothalamus results in a reduction in food intake and altered energy metabolism (Wallis (2020). The Genetic Basis of Obesity and Related Metabolic Diseases in Humans and Companion Animals. Genes, 11(11), 1378). The same mechanism of central action is described for the range of agents regulating Gas-cAMP signaling, the axis triggered by the regulatory peptides of the described group as described above. For instance, both GLP-1 receptor agonists (Peterfi (2021). Glucagon-Like Peptide-1 Regulates the Proopiomelanocortin Neurons of the Arcuate Nucleus both Directly and Indirectly via Presynaptic Action. Neuroendocrinology, 111(10), 986-997.) and AMY2 receptor agonists (Boccia (2020). Amylin brain circuitry. Peptides, 170366.) induce POMC production by POMC neurons and provide regulation of energy metabolism. It should be mentioned that the regulatory peptides of the described group presumably could act on MC4R-expressing neurons providing the effects similar to α-MSH by mimicking intracellular signaling pathways of MC4R which also appears to be a Gas-associated GPCR (Ghamari-Langroudi (2015). G-protein-independent coupling of MC4R to Kir7.1 in hypothalamic neurons. Nature, 520(7545), 94-98). Besides the central effects on appetite and eating behavior, described regulatory peptides could regulate blood glucose levels applying mechanisms similar to leptin. It is known that hypothalamic signaling of leptin through Stat3 is required for the acute effects of leptin on liver glucose fluxes and alleviation of insulin resistance (Buettner (2006). Critical role of STAT3 in leptin's metabolic actions. Cell metabolism, 4(1), 49-60).

In embodiments, the present peptides provide activation of intracellular insulin receptor signaling in peripheral tissues. In embodiments, the present peptides normalize the levels of p-AKT (Thr308) and p-AKT (Ser473) in the liver of rats kept on a high-sucrose diet that correlated with the decrease in the insulin resistance index. pAKT is one of the key participants in the intracellular signaling cascade of the insulin receptor: the insulin response is accompanied by activation of AKT—phosphorylation at Thr308 and Ser473. Activation of AKT is expressed in the induction of several processes: translocation of the glucose transporter GLUT4 to the cell surface (induction of pumping glucose from the blood into tissues), activation of glycogen synthesis by suppressing GSK3 activity, inhibition of gluconeogenesis, activation of the synthesis of fatty acids from glucose. Activation of these processes leads to a decrease in blood sugar levels, the intensity of glucose synthesis, and the induction of its pumping into tissues for further processing. The activation of AKT and the above processes show a strong therapeutic for T2D and other metabolic pathologies (in particular, non-alcoholic fatty liver disease (NAFLD)/non-alcoholic steatohepatitis (NASH)). For pathologies associated with the development of insulin resistance (T2D, NAFLD/NASH), the insulin response is characterized by decreased levels of AKT phosphorylation at Thr308 and Ser473 compared to healthy subjects (Sun et al. (2011) Bioactive Peptides in Milk and Dairy Products: A Review. Korean J Food Sci Anim Resour. 2015; 35(6):831-40; Karlsson et al. (2005) Insulin-Stimulated Phosphorylation of the Akt Substrate AS160 Is Impaired in Skeletal Muscle of Type 2 Diabetic Subjects: Diabetes. 54 (6):1692-1697; Xu et al. (2016) Metformin improves hepatic IRS2/PI3K/Akt signaling in insulin-resistant rats of NASH and cirrhosis. J Endocrinol. 229(2):133-44). In some cases, such a decrease may underlie the development of pathology (Kondapaka et al. (2004) 7-hydroxystaurosporine (UCN-01) inhibition of Akt Thr308 but not Ser473 phosphorylation: a basis for decreased insulin-stimulated glucose transport. Clin Cancer Res. 1; 10(21):7192-8). Decreased pAKT levels are also characteristic of animal models of metabolic pathologies, particularly animals on a high-fat diet (Frosig et al. (2013) AMPK and insulin action--responses to ageing and high fat diet. PLoS One. 6; 8(5):e62338.) and transgenic models (Lee et al. (2019) BRD7 deficiency leads to the development of obesity and hyperglycemia. Sci Rep. 29; 9(1):5327). In embodiments, the regulatory peptides induced the expression level of IRS2 gene in isolated murine fibroblasts. IRS2 is one of the main components of the insulin receptor intracellular signaling pathway, and its tyrosine phosphorylation is associated with increased insulin sensitivity (reduction of insulin resistance) which is considered a positive therapeutic effect. Activation of IRS2 through phosphorylation leads to the induction of glucose transport and increase in the activity of other insulin signaling pathway participants such as PI3 kinase and Akt (Kubota (2017). Imbalanced insulin actions in obesity and type 2 diabetes: key mouse models of the insulin signaling pathway. Cell metabolism, 25(4), 797-810). In the case of IRS2 transcription it is reported that its induction characterizes the changes in the functioning of insulin- and glucose-dependent intracellular pathways in general (Canettieri (2005). Dual role of the coactivator TORC2 in modulating hepatic glucose output and insulin signaling. Cell metabolism, 2(5), 331-338). Induction of IRS2 expression by the regulatory peptides of the described group could be associated with their ability to influence the insulin receptor intracellular signaling pathway.

Drugs aimed to stabilize insulin sensitivity normalize or induce AKT phosphorylation levels. Similar effects have been shown for metformin (Xu et al. (2016) Metformin improves hepatic IRS2/PI3K/Akt signaling in insulin-resistant rats of NASH and cirrhosis. J Endocrinol. 229(2): 133-44), glucagon receptor agonists (Kim et al. (2018) Hepatic glucagon receptor signaling enhances insulin-stimulated glucose disposal in rodents. Diabetes. 67(11): 2157-66), and GLP1 agonists (Tsuboi et al. (2016) The dipeptidyl peptidase IV inhibitor vildagliptin suppresses development of neuropathy in diabetic rodents: effects on peripheral sensory nerve function, structure, and molecular changes. J. Neurochem. 136(4):859-70). Furthermore, AKT is also involved in eating behavior regulation: AKT phosphorylation in the hypothalamus (in particular, induced by Exendin-4) may play an essential role in reducing food consumption (Yang et al. (2017) Exendin-4 reduces food intake via the PI3K/AKT signaling pathway in the hypothalamus. Sci. rep. 7(1):1-7). Thus, the target tissue for the effects promoted by pAKT, apart from the liver, muscles, and adipocytes, also includes the brain. In addition, the occurrence mechanisms of concomitant diabetic symptoms may be of additional interest. In particular, decreased pAKT levels in keratinocytes are associated with increased ulceration in the case of diabetes, and increased phosphorylation levels are associated with increased VEGF production and stimulation of wound healing (Goren et al. (2009) Akt1 controls insulin-driven VEGF biosynthesis from keratinocytes: implications for normal and diabetes-impaired skin repair in mice. J Invest Dermatol. 129(3):752-64). A similar mechanism has been described for retina dysfunction in diabetes (Kim et al. (2017) The Effects of Metformin on Obesity-Induced Dysfunctional Retinas. Invest Ophthalmol Vis Sci. 58(1):106-118).

70S6k1 kinase is an effector of the mTOR cascade. Its activation, particularly Thr421/Ser424 phosphorylation, directly triggers insulin signaling through the IRS/PI3K/AKT cascade (Khamzina et al. (2005) Increased activation of the mammalian target of rapamycin pathway in liver and skeletal muscle of obese rats: possible involvement in obesity-linked insulin resistance. Endocrinol. 146(3):1473-81). In addition to its functions of activating protein biosynthesis, p70S6k1 (Thr421/Ser424) is also involved in a reverse regulatory loop, providing inactivation of IRS1/2 through phosphorylation at Ser636 and Ser639 residues, which inhibits the ability of IRS1/2 to activate PI3K and AKT (Tremblay et al. (2001) A negative feedback mechanism leading to insulin resistance in skeletal muscle cells. J Biol Chem. 276(41):38052-60). Under normal conditions, with a rapid cellular response to insulin, an increased pAKT level correlates with an increased level of p70S6k1 activation, and an inverse regulatory loop provides a gradual attenuation of the cascade. However, under conditions of metabolic pathologies (high-fat diet, type 2 diabetes, etc.), the level of p70S6k1 (Thr421/Ser424) activation is increased in different tissues (e.g., without limitation, the liver and muscles), which leads to hyperactivation of the reverse regulatory loop and disturbances in normal insulin receptor signaling, including a decrease in AKT activation (Khamzina et al. (2005) Increased activation of the mammalian target of rapamycin pathway in liver and skeletal muscle of obese rats: possible involvement in obesity-linked insulin resistance. Endocrinol. 146(3):1473-81; Tremblay et al. (2001) A negative feedback mechanism leading to insulin resistance in skeletal muscle cells. J Biol Chem. 276(41):38052-60; Tremblay et al. (2005) Overactivation of S6 kinase 1 as a cause of human insulin resistance during increased amino acid availability. Diabetes. 54(9):2674-84; Korsheninnikova et al. (2006) Sustained activation of the mammalian target of rapamycin nutrient sensing pathway is associated with hepatic insulin resistance, but not with steatosis, in mice. Diabetologia. 49(12): 3049-57). This process is one of the mechanisms for the development of insulin resistance.

Thus, in embodiments, the present peptides improve insulin signaling by inducing the activity of insulin receptor intracellular pathways participants.

The inventors of the present disclosure describe, inter alia, regulatory peptides with novel structures and having the activity as positive modulators of neuropeptide S receptor (NPSR1), antagonists of FFAR2 (GPR43), and GPR109A receptors, positive modulators of LPAR3 receptor, and inverse agonists of M2 muscarinic receptor. These peptides showed prominent effects on lowering blood glucose, reducing body weight and fat mass in experimental animals, with efficacy similar to or greater than those of metformin. The inventors demonstrated, inter alia, a prominent efficiency of the peptides in reducing appetite and normalizing eating behavior that correlated with activation of Stat3 pathway in the hypothalamus which is considered the main link involved in food consumption control. The regulatory peptides demonstrated high efficiency in reducing insulin resistance index that correlated with induction of the activity of participants of intracellular insulin receptor signaling pathway in the liver. Also, the inventors observed the efficiency of the peptides in reducing general inflammation evoked as a result of a high-fat diet. The effects described above were confirmed by experiments on rodents applying the models of diet-induced obesity and T2D (in particular, high-sucrose diet and high-fat diet). Additionally, the regulatory peptides were able to induce expression of the main participant of intracellular insulin receptor signaling and also to reduce expression levels of the key pro-inflammatory cytokines elevated in the presence of lipopolysaccharide in primary murine fibroblasts in vitro. The positive modulation of NPSR1, antagonistic action on FFAR2 and GPR109A receptors, positive modulation of LPAR3 receptor, and inverse agonistic action on M2 muscarinic receptor by the studied peptides was confirmed using in vitro functional assays performed in the cell lines expressing the respective receptors. The action of the described regulatory peptides through the targets listed above was supported by the ability of the peptides to induce in HepG2 human hepatocellular carcinoma cell line intracellular Gsa-cAMP signaling and Stat3 signaling typical for these targets. The results are discussed in more detail in the Examples below.

The inventors of the present disclosure have discovered and analyzed previously undescribed milk hydrolysate-derived peptides in treating metabolic diseases, such as obesity and diabetes. A set of novel peptides were tested for in vitro activity in murine fibroblasts, and some of them have been shown to enhance IRS2 gene transcription. In aspects, some peptides were chosen after preliminary in vitro screening, and a subset of peptides was further tested in both in vitro and in vivo models of metabolic diseases. In aspects, peptides' activity under stressful conditions was assessed in vitro. It has confirmed that illustrative peptide DLSKEPSISRE (SEQ ID NO: 1) and its pharmacophores SKEPSIS (SEQ ID NO: 2) and EPSI have prominent effects on insulin sensitivity, glucose tolerance, and inflammatory response, and the effects were similar to those observed after metformin treatment, as discussed in more detail below.

The inventors evaluated the efficacy of the illustrative peptide, DLSKEPSISRE (SEQ ID NO: 1), pharmacophores SKEPSIS (SEQ ID NO: 2), and EPSI, as well as tested other (known) test substances, using murine fibroblasts, HepG2 human hepatocellular carcinoma cell line, BALB/c mice, and Sprague-Dawley rats as a model, as discussed in more details below in the Examples section. It is known that metabolic disorders are associated with impaired insulin signaling (Kubota (2017) Imbalanced insulin actions in obesity and T2D: key mouse models of the insulin signaling pathway. Cell Metab. 25(4): 797-810), and inflammation (Hotamisligil (2006) Inflammation and metabolic disorders. Nature. 444: 860-867). In vitro models of stress are a valuable tool in early drug discovery (Hallen (2009). Cell-based in vitro and ex vivo models in metabolic disease drug discovery: nice to have or critical path?Expert Opin Drug Discov. 4(4): 417-428). High glucose in the incubation medium replicates hyperglycemia in diabetic conditions, bacterial lipopolysaccharides (LPS) in the incubation medium induces an inflammatory response in cells, and incubation in serum-free medium imitates cell starvation in various pathological conditions. DLSKEPSISRE (SEQ ID NO: 1) peptide, as well as its pharmacophores SKEPSIS (SEQ ID NO: 2) and EPSI, upregulated IRS2 gene expression levels under normal conditions and stress (inflammation induction and high-glucose medium) in the same manner as metformin. Considering the fact that the induction of IRS2 expression level is typical for the action of the key anti-diabetic agents such as metformin (Ismail (2015). Molecular and immunohistochemical effects of metformin in a rat model of type 2 diabetes mellitus. Experimental and therapeutic medicine, 9(5), 1921-1930.) and GLP-1R agonists (Park (2006). Exendin-4 uses Irs2 signaling to mediate pancreatic β cell growth and function. Journal of Biological Chemistry, 281(2), 1159-1168), this effect of the described group of the peptides could indicate their ability to influence intracellular pathways responsible for the response on insulin and glucose levels. DLSKEPSISRE (SEQ ID NO: 1) peptide as well as its pharmacophores SKEPSIS (SEQ ID NO: 2) and EPSI demonstrated a pronounced anti-inflammatory effect by reducing LPS-induced upregulation of expression of pro-inflammatory cytokines TNF-α and IL-6 similarly to metformin. The described peptides reduced GSK3p gene transcription levels under normal conditions and starvation and inflammatory stress. Increased GSK3p expression is typical for diabetes (Takahashi-Yanaga (2013) Activator or inhibitor? GSK-3 as a new drug target. Biochem. Pharmacol. 86(2): 191-9), and metformin anti-diabetic action is partly associated with downregulation of GSK3p (Sarfstein (2013) Metformin downregulates the insulin/IGF-I signaling pathway and inhibits different uterine serous carcinoma (USC) cells proliferation and migration in p53-dependent or -independent manners. PloS one. 8(4): e61537). Reduction of GSK3p expression by the peptides should be considered as a strong hypoglycemic effect: it should be associated with activation of glycogen synthase, which under food deprivation encourages the cell to use the reserves rather than synthesize glycogen.

The inventors evaluated the ability of the illustrative peptides to induce Stat3 signaling in different tissues, e.g., without limitation, the brain and liver. The present peptides demonstrated the ability to induce the levels of pStat3 in the murine hypothalamus in vivo with an efficacy comparable to leptin used as a positive control. Stat3 signaling pathway in the hypothalamus is considered as the main link involved in the regulation of eating behavior (Kwon (2016). Leptin signaling pathways in hypothalamic neurons. Cellular and Molecular Life Sciences, 73(7), 1457-1477) that reveals the mechanism of appetite regulation by the bioactive peptides of the present disclosure. In embodiments, the regulatory peptides were able to induce Stat3 signaling in HepG2 human cell line of hepatic origin that indicates the comprehensive mechanism of action.

In embodiments, the present peptides were able to regulate appetite and reduce food consumption in rodents. This effect of the regulatory peptide corresponds to NPSR1 as their main target and activation of Stat3 signaling in the hypothalamus.

In embodiments, the present peptides were able to activate cellular Stat3 signaling applying Gsa protein in hepatic cells. This effect refers to the action of the peptides of the present disclosure as positive modulators of NPSR1 and antagonists of FFAR2 and GPR109A.

In embodiments, the present peptides provided pronounced hypoglycemic effect in the glucose tolerance test after single administration in rodent models of diet-induced obesity and T2D. This effect indicates the ability of the peptides of the present disclosure to normalize glucose levels in the subjects with metabolic disorders, which is considered a key therapeutic effect of anti-diabetic medications (Chaudhury (2017). Clinical review of antidiabetic drugs: implications for type 2 diabetes mellitus management. Frontiers in endocrinology, 8, 6).

In embodiments, chronic administration of the present peptides to the rodents with diet-induced metabolic disorders resulted in significantly reduced blood glucose levels compared to unhealthy control.

In embodiments, the present peptides reduced insulin concentration and insulin resistance index values in rodent models of diet-induced obesity and type 2 diabetes to healthy control levels, both as a result of single and chronic administration. This effect indicates the ability of the peptides of the present disclosure to alleviate insulin resistance conditions and to normalize insulin signaling.

In embodiments, the present peptides significantly reduced the bodyweight of the rodents with characteristics of metabolic disorders caused by a chronic high-fat diet. This effect indicates the peptides as effective agents for obesity treatment.

In embodiments, the present peptides significantly decreased visceral fat mass index and the linear size of adipocytes in the rodents with characteristics of metabolic disorders caused by a chronic high-fat diet. This effect indicates the peptides as effective agents for obesity treatment and the prevention of cardiovascular diseases.

In embodiments, the present peptides significantly reduced general inflammatory response estimated by blood levels of pro-inflammatory cytokine TNF-α in the rodents kept on a chronic high-fat diet to the levels of healthy control. Because chronic inflammation may represent a triggering factor in the origin and development of metabolic disorders (Esposito (2004). The metabolic syndrome and inflammation: association or causation?Nutrition, Metabolism and Cardiovascular Diseases, 14(5), 228-232.), this effect could indicate the protective function of the present peptides affecting one of the key mechanisms of metabolic disorders initiation and progression.

In embodiments, the present peptides induced the levels of participants of intracellular insulin receptor signaling pathway in the liver of the rodents with characteristics of metabolic disorders caused by a chronic high-fat diet. This effect refers to the ability of the peptides of the present disclosure to improve insulin signaling and alleviate insulin resistance conditions

The inventors of the present disclosure discovered and evaluated bioactive peptides that were estimated to have the activity as positive modulators of neuropeptide S receptor (NPSR1), antagonists of FFAR2 (GPR43) and GPR109A receptors, positive modulators of LPAR3 receptor, and inverse agonists of M2 muscarinic receptor and to have the activity profile of anti-diabetic and anti-inflammatory medications.

In aspects, the present disclosure provides a composition comprising a synthetic peptide, the synthetic peptide comprising or consisting of an amino acid sequence derivable or derived from one or more milk hydrolysate proteins, wherein the peptide is capable of modulating metabolism.

In embodiments, the synthetic peptide comprises or consists of about 4 to about 12 amino acids. In embodiments, the synthetic peptide comprises or consists of about 4 to about 8 amino acids. In embodiments, the synthetic peptide comprises or consists of about 12 amino acids, or about 11 amino acids, or about 10 amino acids, or about 9 amino acids, or about 8 amino acids, or about 7 amino acids, or about 6 amino acids, or about 5 amino acids, or about 4 amino acids.

In aspects, the inventors of the present disclosure conducted various experiments in cell cultures and animal models as described herein, and, as a result, a group of peptides defined by a following general formula was identified: X₁X₂X₃X₄R₁R₂R₃R₄Y₁Y₂Y₃ (I).

In embodiments, X₁ is absent or a polar and negatively charged hydrophilic amino acid, optionally selected from aspartate (D) and glutamate (E);
X₂ is absent or a hydrophobic, aliphatic amino acid, optionally selected from glycine (G), alanine (A), leucine (L), isoleucine (I), methionine (M), and valine (V);
X₃ is absent or a polar and neutral charged hydrophilic amino acid, optionally selected from asparagine (N), glutamine (Q), serine (S), threonine (T), proline (P), and cysteine (C);
X₄ is absent or a polar and positively charged hydrophilic amino acid, optionally selected from arginine (R) and lysine (K);
R₁ is selected from a polar and negatively charged hydrophilic amino acid, optionally selected from aspartate (D) and glutamate (E);
R₂ is selected from a polar and neutral charged hydrophilic amino acid, optionally selected from asparagine (N), glutamine (Q), serine (S), threonine (T), proline (P), and cysteine (C);
R₃ is selected from a polar and neutral charged hydrophilic amino acid, optionally selected from asparagine (N), glutamine (Q), serine (S), threonine (T), proline (P), and cysteine (C);
R₄ is selected from a hydrophobic, aliphatic amino acid, optionally selected from glycine (G), alanine (A), leucine (L), isoleucine (I), methionine (M), and valine (V);
Y₁ is absent or a polar and neutral charged hydrophilic amino acid, optionally selected from asparagine (N), glutamine (Q), serine (S), threonine (T), proline (P), and cysteine (C);
Y₂ is absent or a polar and positively charged hydrophilic amino acid, optionally selected from arginine (R) or lysine (K); and
Y₃ is absent a polar and negatively charged hydrophilic amino acid, optionally selected from aspartate (D) and glutamate (E).

In embodiments, X₁ is aspartate (D).

In embodiments, X₂ is leucine (L).

In embodiments, X₃ is serine (S).

In embodiments, X₄ is lysine (K).

In embodiments, R₁ is glutamate (E).

In embodiments, R₂ is proline (P).

In embodiments, R₃ is serine (S).

In embodiments, R₄ is isoleucine (1).

In embodiments, Y₁ is serine (S).

In embodiments, Y₂ is arginine (R).

In embodiments, Y₃ is glutamate (E).

In embodiments, R₁ is glutamate (E); R₂ is proline (P); R₃ is serine (S); and R₄ is isoleucine (1).

In embodiments, X₃ is serine (S); X₄ is lysine (K); R₁ is glutamate (E); R₂ is proline (P);

R₃ is serine (S); R₄ is isoleucine (I); and Y₁ is serine (S).

In embodiments, X₁ is aspartate (D); X₂ is leucine (L); X₃ is serine (S); X₄ is lysine (K); R₁ is glutamate (E); R₂ is proline (P); R₃ is serine (S); R₄ is isoleucine (1); Y₁ is serine (S); Y₂ is arginine (R); and Y₃ is glutamate (E).

In embodiments, the synthetic peptide consists of amino acids that do not include an aromatic, polar and positively charged hydrophilic amino acid, optionally a histidine (H).

In embodiments, the synthetic peptide consists of amino acids that do not include a hydrophobic, aromatic amino acid, optionally selected from phenylalanine (F), tryptophan (W), and tyrosine (Y).

In embodiments, the peptide is chemically modified. In embodiments, a peptide in accordance with the present disclosure is an active ingredient of the composition. In other embodiments, the active ingredient of the composition is an analog of the peptide, which is an N-terminal modified analog or a C-terminal modified analog. The peptide in accordance with the present disclosure is optionally chemically modified. In embodiments, the chemical modification is selected from amidation, methylation, and acetylation of one or more of X₁X₂X₃X₄R₁R₂R₃R₄Y₁Y₂Y₃, as described herein for Formula I. In other embodiments, other various types of peptide backbone and/or side chain modifications are performed. In embodiments, chemical modifications can include addition of formyl, pyroglutamyl (pGlu), one or more fatty acids, urea, carbamate, sulfonamide, alkylamine, or any combination thereof, to one or more of X₁, X₂, X₃, X₄, R₁, R₂, R₃, R₄, Y₁, Y₂, and Y₃, as described herein for Formula I.

In embodiments, the synthetic peptide is a “pseudo-peptide” where the regular peptide bond (CO—NH) is replaced with one of an isosteric or isoelectronic analog. For example, the reduced amide (CH2-NH) is isosterically introduced into the peptide. In embodiments, the peptide is made in the form of azapeptide, where α-Carbon of the peptide backbone is replaced with nitrogen (without changing the amino acids residues). As a further example of a chemical modification, the synthetic peptide in accordance with the present disclosure is a retro-inverso peptide where a D-amino acid is used in a reversed sequence. As yet another example, in embodiments, the synthetic peptide in accordance with the present disclosure is peptidomimetic having its side chains appended to the nitrogen atom of the peptide backbone, rather than to the α-carbons. In this way, the synthetic peptide is, in embodiments, a peptoid, or poly-N-substituted glycine.

In embodiments, the synthetic peptide is optionally modified by incorporating non-natural amino acids into certain positions in the peptide. Non-limiting examples of the non-natural amino acids include D-amino acids, N-methylated (or N-alkylated) amino acids, alpha-substituted alpha-amino acids, beta-substituted alpha-amino acids, beta-amino acids, and gamma-amino acids.

In embodiments, the synthetic peptide is modified by cyclization of the peptide. In embodiments, the synthetic peptide is modified such that the peptide is a beta-turn mimetics peptide. In embodiments, phenylalanine (F) in the peptide, if present, is replaced with nitro-, amino-, fluoro-phenylalanine, or other inhibitors of proteases.

In embodiments, the chemical modification is selected from amidation, methylation, and acetylation of one or more of X₁, X₂, X₃, X₄, R₁, R₂, R₃, R₄, Y₁, Y₂, and Y₃, as described herein for Formula I.

In embodiments, the chemical modification is selected from addition of formyl, pyroglutamyl (pGlu), a fatty acid, urea, carbamate, sulfonamide, alkylamine, or any combination thereof, to one or more of X₁, X₂, X₃, X₄, R₁, R₂, R₃, R₄, Y₁, Y₂, and Y₃, as described herein for Formula I.

In embodiments, the chemical modification incorporates non-natural amino acids into the peptide.

In embodiments, the non-natural amino acids are selected from D-amino acids, N-methylated (or N-alkylated) amino acids, alpha-substituted alpha-amino acids, beta-substituted alpha-amino acids, beta-amino acids, and gamma-amino acids.

In embodiments, the composition further comprises a pharmaceutically acceptable carrier.

In embodiments, the composition further comprises a delivery vehicle. In embodiments, the delivery vehicle is selected from a liposome, a nanoparticle, and a polysaccharide. In embodiments, the polysaccharide is selected from cyclodextrin, chitosan, cellulose, and alginate.

The composition in accordance with the present disclosure is formulated for various routes of administration. Non-limiting examples of routes of administration include inhalation, intranasal, oral, intravenous, intramuscular, and subcutaneous.

In embodiments, the composition is formulated for intranasal administration. In embodiments, the composition comprises at least one inhibitor of nasal mucosa proteases. In embodiments, the inhibitor is selected from bestatine, comostate amylase, leupeptin, aprotinin, bacitracin, amastatine, boroleucine, puromycin, a bile salt, and a fusidic acid (e.g., without limitation, disodium ethylene-diaminetetraacetate). Intranasal delivery is a non-invasive route of administration for the therapeutic peptides and provides an alternative to intravenous or subcutaneous injections.

In embodiments, the composition is formulated for administration by inhalation. In embodiments, the composition formulated for administration by inhalation is administered using an intranasal device. The intranasal device is, for example, a dry powder intranasal device configured to deliver a therapeutic agent to a subject in the form of a dry powder. In embodiments, the intranasal device is configured for use outside of a clinical setting, such that a therapeutic agent is self-administered by a subject.

In embodiments, the composition is formulated for intravenous administration. In embodiments, the composition is formulated for oral administration. In embodiments, the composition is formulated for parenteral administration. In embodiments, the composition is formulated for subcutaneous administration.

In embodiments, the composition is formulated for intramuscular administration. In embodiments, the composition is formulated for sublingual or buccal administration. In embodiments, the composition is formulated for intradermal administration. In embodiments, the composition is formulated for transdermal administration.

In embodiments, the present disclosure provides a pharmaceutical composition comprising a therapeutically effective amount of the composition of the present disclosure and at least one pharmaceutically acceptable carrier, diluent, or excipient.

In embodiments, the synthetic peptide is a regulatory peptide. In embodiments, the synthetic peptide is a biologically active peptide.

In embodiments, the synthetic peptide is capable of modulating neuropeptide S receptor 1 (NPSR1)

In embodiments, the synthetic peptide is an antagonist of free fatty acid receptor 2 (FFAR2).

In embodiments, the synthetic peptide is an antagonist of G-protein-coupled receptor 43 (GPR43).

In embodiments, the synthetic peptide is an antagonist of G protein-coupled receptor 109A (GPR109A).

In embodiments, the synthetic peptide is a positive allosteric modulator of lysophosphatidic acid receptor 3 (LPAR3).

In embodiments, the synthetic peptide is an inverse agonist of muscarinic acetylcholine receptor 2 subtype (M2).

In embodiments, the synthetic peptide induces stimulatory G protein α-subunit (Gsa)-cAMP axis in different tissues, optionally resulting in activation of intracellular Stat3 signaling. In embodiments, the synthetic peptide induces intracellular Stat3 signaling. In embodiments, the synthetic peptide induces intracellular Stat3 signaling in the brain, optionally in the hypothalamus.

In embodiments, the synthetic peptide modulates appetite regulation, glucose homeostasis, insulin resistance, and/or fat mass decrease.

In embodiments, the synthetic peptide aregulates expression of genes involved in the pathogenesis of various metabolic conditions.

In embodiments, the synthetic peptide activates insulin receptor substrate 2 (IRS2) gene expression.

In embodiments, the synthetic peptide triggers a downstream anti-inflammatory effect.

In embodiments, the synthetic peptide reduces expression levels of one or more pro-inflammatory cytokines elevated in the presence of lipopolysaccharide (LPS), optionally selected from interleukin 6 (IL-6) and tumor necrosis factor α (TNFα).

In embodiments, the synthetic peptide lowers blood glucose and/or reduces body weight and fat mass. In embodiments, the synthetic peptide regulates appetite and/or an eating behavior. In embodiments, the synthetic peptide reduces an insulin resistance index. In embodiments, the synthetic peptide reduces general inflammation, optionally due to a high-fat diet. In embodiments, the synthetic peptide modulates insulin sensitivity, glucose tolerance, and/or inflammatory response.

In embodiments, the synthetic peptide normalizes glucose levels in a subject with a metabolic disorder. In embodiments, the synthetic peptide reduces insulin concentration in a subject when administered. In embodiments, the synthetic peptide reduces insulin resistance index values in a subject when administered.

In embodiments, the synthetic peptide alleviates insulin resistance conditions and/or normalizes insulin signaling when administered.

In embodiments, the synthetic peptide reduces the body weight of a subject when administered. In embodiments, the synthetic peptide decreased visceral fat mass index and the linear size of adipocytes in a subject when administered.

In embodiments, the present disclosure provides a food product comprising the synthetic peptide of the present disclosure, wherein the synthetic peptide is an active ingredient in the food product. In embodiments, the food product is selected from bars, shakes, juices, yogurts, drinks, or the like. In embodiments, the food composition includes any non-active ingredients.

In embodiments, the peptide, or more than one peptide, in accordance with the present disclosure is included as an active ingredient in a foodstuff. In these embodiments, the peptide is included in a composition that is a food preparation. The food composition can include any non-active ingredients. Furthermore, the food composition can include, in addition to the peptide(s) in accordance with the present disclosure, other active ingredients that do not affect the effectiveness of the peptide.

In embodiments, the present disclosure provides a method for treating a related condition in a patient in need thereof, comprising administering a therapeutically effective amount of the composition of the present disclosure to a patient in need thereof.

In embodiments, the condition is a metabolic disease or disorder.

In embodiments, the condition is an NPSR1-mediated condition.

In embodiments, the condition is selected from diabetes mellitus (DM) (optionally, selected from Type 1 diabetes, Type 2 diabetes, hybrid form of diabetes (optionally, selected from immune-mediated diabetes of adults, ketosis-prone type 2 diabetes), hyperglycemia first detected during pregnancy (optionally, selected from DM in pregnancy and gestational DM)), intermediate hyperglycemia (optionally, selected from impaired fasting glucose, impaired glucose tolerance, other specified intermediate hyperglycemia, and unspecified intermediate hyperglycemia), another insulin-resistance syndrome, other specified or unspecified disorders of glucose regulation and pancreatic internal secretion, overweight (optionally, selected from overweight in infants, children or adolescents, overweight in adults, and localized adiposity), obesity (optionally, selected from obesity due to energy imbalance including but not limited by obesity in children or adolescents and obesity in adults, drug-induced obesity, obesity hypoventilation syndrome, Prader-Willi syndrome, other specified obesity, and unspecified obesity), feeding or eating disorders (optionally, selected from bulimia nervosa, binge eating disorder, and other specified feeding or eating disorders), non-alcoholic fatty liver disease (optionally, selected from non-alcoholic fatty liver disease without non-alcoholic steatohepatitis and non-alcoholic steatohepatitis), hyperlipoproteinaemia (optionally, selected from hypercholesterolaemia, hypertriglyceridaemia, mixed hyperlipidaemia, and other specified hyperlipoproteinaemia), and inborn errors of metabolism (optionally, selected from inborn errors of carbohydrate metabolism, inborn errors of lipid metabolism, and inborn errors of energy metabolism).

In embodiments, the diabetes is selected from monogenic diabetes, disease of the exocrine pancreas, endocrine disorders, drug- or chemical-induced diabetes, infection-related diabetes, uncommon specific forms of immune-mediated diabetes, and other genetic syndromes sometimes associated with diabetes.

In embodiments, the metabolic disorder is type 2 diabetes. In embodiments, the metabolic disorder is feeding or eating disorder.

In embodiments, the metabolic disorder is intermediate hyperglycemia selected from impaired fasting glucose, impaired glucose tolerance, other specified intermediate hyperglycemia or unspecified intermediate hyperglycemia.

In embodiments, the metabolic disorder is an insulin-resistance syndrome, or other specified disorders of glucose regulation and pancreatic internal secretion, or unspecified disorders of glucose regulation and pancreatic internal secretion.

In embodiments, the metabolic disorder is overweight or obesity. In embodiments, the metabolic disorder is feeding or eating disorder.

In embodiments, the metabolic disorder is non-alcoholic fatty liver disease optionally selected from non-alcoholic fatty liver disease without non-alcoholic steatohepatitis and non-alcoholic steatohepatitis.

In embodiments, the metabolic disorder is hyperlipoproteinaemia optionally selected from hypercholesterolaemia, hypertriglyceridaemia, mixed hyperlipidaemia and other specified hyperlipoproteinaemia.

In embodiments, the metabolic disorder is an inborn error of metabolism optionally selected from inborn errors of carbohydrate metabolism, inborn errors of lipid metabolism, inborn errors of energy metabolism.

In embodiments, the synthetic peptide is administered in combination with at least one additional therapeutic agent.

In embodiments, the present disclosure provides a method for modulating one or more of NPSR1 receptor, GPR109A (HCAR2) receptor, FFAR2 receptor, CHRM2 receptor, and LPAR3 receptor in a cell by contacting the cell with the composition of the present disclosure.

The regulatory peptide in accordance with the present disclosure is in the form of a pharmaceutical composition. The composition is administered to a subject in need of treatment, e.g., without limitation, a subject diagnosed with a disorder manifesting in diabetes and/or obesity and/or other metabolic condition.

In embodiments, the peptide modulates the NPSR1 receptor, GPR109A (HCAR2) receptor, FFAR2 receptor, CHRM2 receptor, or LPAR3 receptor.

In embodiments, a pharmaceutical composition is provided in accordance with any of the embodiments or any combination of the embodiments described herein, the pharmaceutical composition comprising a therapeutically effective amount of the composition and at least one pharmaceutically acceptable carrier, diluent, or excipient.

In embodiments, a method for modulating NPSR1 receptor, GPR109A (HCAR2) receptor, FFAR2 receptor, CHRM2 receptor, or LPAR3 receptor in a cell is provided. The method comprises contacting the cell with the composition in accordance with any of the embodiments or any combination of the embodiments described herein.

In embodiments, a method for treating a metabolic disorder in a patient in need thereof is provided, the method comprising administering a therapeutically effective amount of the composition in accordance with any of the embodiments described herein to a patient in need thereof. The metabolic disorder is diabetes mellitus (DM). In embodiments, the diabetes is selected from Type 1 diabetes, Type 2 diabetes, a hybrid form of diabetes (immune-mediated diabetes of adults, ketosis-prone type 2 diabetes), hyperglycemia first detected during pregnancy (DM in pregnancy, gestational DM). In embodiments, diabetes is other specific types of diabetes: monogenic diabetes, disease of the exocrine pancreas, endocrine disorders, drug- or chemical-induced diabetes, infection-related diabetes, uncommon specific forms of immune-mediated diabetes, and other genetic syndromes sometimes associated with diabetes.

In embodiments, the metabolic disorder is intermediate hyperglycemia including but not limited by impaired fasting glucose, impaired glucose tolerance, other specified intermediate hyperglycemia, or unspecified intermediate hyperglycemia.

In embodiments, the metabolic disorder is another insulin-resistance syndrome, or other specified disorders of glucose regulation and pancreatic internal secretion, or unspecified disorders of glucose regulation and pancreatic internal secretion.

In embodiments, the metabolic disorder is overweight or obesity. In embodiments, overweight is selected from overweight in infants, children, or adolescents, overweight in adults, or localized adiposity. In embodiments, obesity is represented as obesity due to energy imbalance including but not limited to obesity in children or adolescents and obesity in adults. In embodiments, the obesity is drug-induced obesity, obesity hypoventilation syndrome, Prader-Willi syndrome, other specified obesity, and unspecified obesity. Other specified obesity includes but is not limited to obesity due to melanocortin-4 receptor deficiency and leptin-related genetic obesity.

In embodiments, the metabolic disorder is a feeding or eating disorder. In embodiments feeding or eating disorder is selected from bulimia nervosa, binge eating disorder, or other specified feeding or eating disorders.

In embodiments, the metabolic disorder is non-alcoholic fatty liver disease including but not limited to non-alcoholic fatty liver disease without non-alcoholic steatohepatitis and non-alcoholic steatohepatitis.

In embodiments, the metabolic disorder is hyperlipoproteinaemia including but not limited to hypercholesterolaemia, hypertriglyceridaemia, mixed hyperlipidaemia and other specified hyperlipoproteinaemia.

In embodiments, the metabolic disorder is inborn errors of metabolism including but not limited by inborn errors of carbohydrate metabolism, inborn errors of lipid metabolism, inborn errors of energy metabolism.

In embodiments, the present disclosure provides a method of treating DM in a patient in need thereof comprising administering an effective amount of a composition comprising a regulatory peptide. In embodiments, the regulatory peptide is administered in combination with an additional therapeutic agent.

In embodiments, the present disclosure provides a method of treating intermediate hyperglycemia in a patient in need thereof comprising administering an effective amount of a composition comprising a regulatory peptide. In embodiments, the regulatory peptide is administered in combination with an additional therapeutic agent.

In embodiments, the present disclosure provides a method of treating another insulin-resistance syndrome, or other specified disorders of glucose regulation and pancreatic internal secretion in a patient in need thereof comprising administering an effective amount of a composition comprising a regulatory peptide. In embodiments, the regulatory peptide is administered in combination with an additional therapeutic agent.

In embodiments, the present disclosure provides a method of treating overweight or obesity in a patient in need thereof comprising administering an effective amount of a composition comprising a regulatory peptide. In embodiments, the regulatory peptide is administered in combination with an additional therapeutic agent.

In embodiments, the present disclosure provides a method of treating feeding or eating disorder in a patient in need thereof comprising administering an effective amount of a composition comprising a regulatory peptide. In embodiments, the regulatory peptide is administered in combination with an additional therapeutic agent.

In embodiments, the present disclosure provides a method of treating non-alcoholic fatty liver disease in a patient in need thereof comprising administering an effective amount of a composition comprising a regulatory peptide. In embodiments, the regulatory peptide is administered in combination with an additional therapeutic agent.

In embodiments, the present disclosure provides a method of treating hyperlipoproteinaemia in a patient in need thereof comprising administering an effective amount of a composition comprising a regulatory peptide. In embodiments, the regulatory peptide is administered in combination with an additional therapeutic agent.

In embodiments, the present disclosure provides a method of treating inborn errors of metabolism in a patient in need thereof comprising administering an effective amount of a composition comprising a regulatory peptide. In embodiments, the regulatory peptide is administered in combination with an additional therapeutic agent.

In embodiments, the present disclosure includes treatment of DM and/or the symptoms thereof. DM is a disorder characterized by fasting plasma glucose ≥7.0 mmol/L or 2-hour post-load plasma glucose ≥11.1 mmol/L or Hba1c ≥48 mmol/mol, and usually accompanied by frequent urination, increased thirst, and increased appetite. Acute complications of DM can include e.g., without limitation, diabetic ketoacidosis, hyperosmolar hyperglycemic state, or death. Serious long-term complications include e.g., without limitation, cardiovascular disease, stroke, chronic kidney disease, foot ulcers, damage to the nerves, damage to the eyes, and cognitive impairment.

Accordingly, the methods and compositions of the present disclosure are useful for the treatment of DM and/or the symptoms thereof. Any type of DM may be treated using the methods and compositions of the disclosure.

In embodiments, the present disclosure is useful for the treatment of DM, or intermediate hyperglycemia, or another insulin-resistance syndrome combined with overweight or obesity, feeding or eating disorder, non-alcoholic fatty liver disease, hyperlipoproteinaemia, inborn errors of metabolism, or any combinations of these diseases in the same subject. In embodiments, the present disclosure provides a method for treating DM, or intermediate hyperglycemia, or another insulin-resistance syndrome, or overweight or obesity, or feeding or eating disorder, or non-alcoholic fatty liver disease, or hyperlipoproteinaemia, or inborn errors of metabolism, or any combinations of these diseases by administering an effective amount of a composition comprising a regulatory peptide to a patient in need thereof. The patient may also receive pre-existent and/or combination therapy that comprises one or more of the additional therapeutic agents described herein.

A non-limiting example of therapeutic agent used for treatment of metabolic disorders include sodium-glucose transport protein 2 (SGLT2) inhibitors.

Non-limiting examples of therapeutic agents used for treatment of obesity include bupropion-naltrexone (CONTRAVE), Liraglutide (SAXENDA), Orlistat (ALLI and XENICAL), Phentermine-topiramate (QSYMIA), phentermine (ADIPEX and LOMAIRA), lorcaserin (BELVIQ), semaglutide (WEGOVY), setmelanotide (IMCIVREE), and/or other medications that suppress the desire to eat (e.g., without limitation, phentermine, benzphetamine, diethylpropion, and phendimetrazine).

Non-limiting examples of therapeutic agents used for treatment of type 2 diabetes (T2D) include alpha-glucosidase inhibitors (e.g., without limitation, acarbose (PRECOSE) and miglitol (GLYSET)), biguanides (e.g., without limitation, metformin, metformin-canagliflozin (INVOKAMET), metformin-dapagliflozin (XIGDUO XR), metformin-empagliflozin (SYNJARDY), metformin-glipizide, metformin-glyburide (GLUCOVANCE), metformin-linagliptin (JENTADUETO), metformin-pioglitazone (ACTOPLUS), metformin-repaglinide (PRANDIMET), metformin-rosiglitazone (AVANDAMET), metformin-saxagliptin (KOMBIGLYZE XR), and metformin-sitagliptin (JANUMET)), Dopamine agonist (e.g., without limitation, bromocriptine (CYCLOSET)), dipeptidyl peptidase-4 (DPP-4) inhibitors (e.g., without limitation, alogliptin (NESINA), alogliptin-metformin (KAZANO), alogliptin-pioglitazone (OSENI), linagliptin (TRADJENTA), linagliptin-empagliflozin (GLYXAMBI), linagliptin-metformin (JENTADUETO), saxagliptin (ONGLYZA), saxagliptin-metformin (KOMBIGLYZE XR), sitagliptin (JANUVIA), sitagliptin-metformin (JANUMET and JANUMET XR), and sitagliptin and simvastatin (JUVISYNC), glucagon-like peptide-1 receptor agonists (GLP-1 receptor agonists) (e.g., without limitation, albiglutide (TANZEUM), dulaglutide (TRULICITY), exenatide (BYETTA), exenatide extended-release (BYDUREON), liraglutide (VICTOZA), and semaglutide (OZEMPIC)), meglitinides (e.g., without limitation, nateglinide (STARLIX), repaglinide (PRANDIN), and repaglinide-metformin (PRANDIMET)), sodium-glucose transporter (SGLT) 2 inhibitors (e.g., without limitation, dapagliflozin (FARXIGA), dapagliflozin-metformin (XIGDUO XR), canagliflozin (INVOKANA), canagliflozin-metformin (INVOKAMET), empagliflozin (JARDIANCE), empagliflozin-linagliptin (GLYXAMBI), empagliflozin-metformin (SYNJARDY), ertugliflozin (STEGLATRO)), sulfonylureas (e.g., without limitation, glimepiride (AMARYL), glimepiride-pioglitazone (DUETACT), glimepiride-rosiglitazone (AVANDARYL), Gliclazide, glipizide (GLUCOTROL), glipizide-metformin (METAGLIP), glyburide (DIABETA, GLYNASE, MICRONASE), glyburide-metformin (GLUCOVANCE), chlorpropamide (DIABINESE), tolazamide (TOLINASE), and tolbutamide (ORINASE, TOL-TAB)), thiazolidinediones (e.g., without limitation, rosiglitazone (AVANDIA), rosiglitazone-glimepiride (AVANDARYL), rosiglitazone-metformin (AMARYL M), pioglitazone (ACTOS), pioglitazone-alogliptin (OSENI), pioglitazone-glimepiride (DUETACT), and pioglitazone-metformin (ACTOPLUS MET, ACTOPLUS MET XR)).

Non-limiting examples of therapeutic agents used for treatment of eating disorders include olanzapine (ZYPREXA), fluoxetine (PROZAC), topiramate (TOPAMAX); lisdexamfetamine (VYVANSE), and bupropion (WELLBUTRIN).

In embodiments, the present disclosure provides compositions and methods in accordance with any of the described embodiments that further comprise an additional agent and methods of administering the additional agent to a subject. In embodiments, the present disclosure pertains to co-administration and/or co-formulation. Any of the compositions described herein may be co-formulated and/or co-administered with one or more suitable agents.

In embodiments, the additional agent may be conjugated to the peptides in accordance with the present disclosure.

In embodiments, a method for treating type 2 diabetes (T2D) in accordance with any of the embodiments or any combination of the embodiments described herein is provided, the method further comprising administering an additional therapeutic agents used for treatment of type 2 diabetes optionally selected from one or more of metformin, metformin-canagliflozin (INVOKAMET), metformin-dapagliflozin (XIGDUO XR), metformin-empagliflozin (SYNJARDY), metformin-glipizide, metformin-glyburide (GLUCOVANCE), metformin-linagliptin (JENTADUETO), metformin-pioglitazone (ACTOPLUS), metformin-repaglinide (PRANDIMET), metformin-rosiglitazone (AVANDAMET), metformin-saxagliptin (KOMBIGLYZE XR), and metformin-sitagliptin.

In embodiments, a method for treating eating disorders in accordance with any of the embodiments or any combination of the embodiments described herein is provided, the method further comprising administering an additional therapeutic agents used for treatment of eating disorders optionally selected from one or more of olanzapine (ZYPREXA) and fluoxetine (PROZAC).

In embodiments, the present compositions may be fused to other moieties, e.g., without limitation, an additional agent or a moiety to extend half-life in vivo. Apart from increasing stability, such moieties may also increase solubility of the molecule they are fused to. A moiety that increases solubility (e.g., without limitation, prevents aggregation) may provide easier handling of the compositions, and particularly improve stability and shelf-life. A well-known example of such moiety is PEG (polyethylene glycol). This moiety is particularly envisaged, as it is used as linker as well as solubilizing moiety. Other examples include peptides and proteins or protein domains, or even whole proteins (e.g., without limitation, GFP). In this regard, it should be noted that, like PEG, one moiety can have different functions or effects. For instance, a flag tag (DYKDDDDK; SEQ ID NO: 3) is a peptide moiety that is used as a label, but due to its charge density, it will also enhance solubilization. PEGylation has already often been demonstrated to increase solubility of biopharmaceuticals (e.g., without limitation, Veronese and Mero (2008) The impact of PEGylation on biological therapies, BioDrugs.; 22(5)315-29). Adding a peptide, polypeptide, protein, or protein domain tag to a molecule of interest has been extensively described in the art. Examples include, but are not limited to, peptides derived from synuclein (e.g., without limitation, Park et al., Protein Eng. Des. Sel. 2004; 17:251-260), SET (solubility enhancing tag, Zhang et al., Protein Expr Purif 2004; 36:207-216), thioredoxin (TRX), Glutathione-S-transferase (GST), Maltose-binding protein (MBP), N-Utilization substance (NusA), small ubiquitin-like modifier (SUMO), ubiquitin (Ub), disulfide bond C (DsbC), Seventeen kilodalton protein (Skp), Phage T7 protein kinase fragment (T7PK), Protein G BI domain, Protein A IgG ZZ repeat domain, and bacterial immunoglobulin binding domains (Hutt et al., J Biol Chem.; 287(7):4462-9, 2012). The nature of the tag will depend on the application, as determined by the skilled person. For instance, for transgenic expression of the molecules described herein, it might be envisaged to fuse the molecules to a larger domain to prevent premature degradation by the cellular machinery. Other applications may envisage fusion to a smaller solubilization tag (e.g., without limitation, less than 30 amino acids, or less than 20 amino acids, or even less than 10 amino acids) in order not to alter the properties of the molecules too much. Additional chemical modifications can include, e.g., without limitation, addition of formyl, pyroglutamyl (pGlu), one or more fatty acids, urea, carbamate, sulfonamide, alkylamine, or any combination thereof.

Apart from extending half-life, the present compositions (e.g., without limitation, one or more peptides in accordance with embodiments of the present disclosure) may be fused to moieties that alter other or additional pharmacokinetic and pharmacodynamic properties. For instance, it is known that fusion with albumin (e.g., without limitation, human serum albumin), albumin-binding domain or a synthetic albumin-binding peptide improves pharmacokinetics and pharmacodynamics of different therapeutic proteins (Langenheim and Chen, Endocrinol.; 203(3):375-87, 2009). Another moiety that is often used is a fragment crystallizable region (Fc) of an antibody. The nature of these moieties are determined by the person skilled in the art depending on the application.

In embodiments, the peptides of the present disclosure are administered as the sole pharmaceutical agent or in combination with one or more other pharmaceutical agents where the combination causes no unacceptable adverse effects.

The amount of the active ingredient to be administered in the treatment of one or more conditions can vary according to such considerations as the particular peptide and dosage unit employed, the mode of administration, the period of treatment, the age, weight, and sex of the patient treated, and the nature and extent of the condition treated. The composition in accordance with the present disclosure is administered to a subject at the appropriate dose via a certain route.

In embodiments, a dose of the peptide to be administered will generally range from about 0.001 mg/kg to about 200 mg/kg body weight, from about 0.01 mg/kg to about 100 mg/kg body weight, from about 0.01 mg/kg to about 50 mg/kg body weight, from about 0.01 mg/kg to about 40 mg/kg body weight, from about 0.01 mg/kg to about 30 mg/kg body weight, from about 0.01 mg/kg to about 20 mg/kg body weight, from about 0.01 mg/kg to about 5 mg/kg body weight, from about 0.01 mg/kg to about 10 mg/kg body weight, from about 0.1 mg/kg to about 10 mg/kg body weight, from about 0.1 mg/kg to about 20 mg/kg body weight, from about 0.1 mg/kg to about 30 mg/kg body weight, from about 0.1 mg/kg to about 40 mg/kg body weight, from about 0.1 mg/kg to about 50 mg/kg body weight. Clinically useful dosing schedules will range from one to three times a day dosing. A pharmaceutical composition with the regulatory peptides described herein can also be administered as a single dose. Because of the safety and effectiveness of the composition, the single dose of the composition is effective in alleviating symptoms of metabolic disorders. Treatment schedules can also be developed for a more prolonged treatment course. For example, in embodiments, a pharmaceutical composition in accordance with embodiments of the present disclosure is administered during more than one day, for instance, from 2 days to 60 days, or from 2 days to 50 days, or from 2 days to 40 days, or from 2 days for 30 days, and the daily dose is within any of the above ranges. The administration for more than one day is used for the treatment of chronic symptoms or disorders, which is any of various metabolic disorders, including but not limited to type 2 diabetes, prediabetes (intermediate hyperglycemia), and obesity.

A “subject” is a mammal, e.g., a human (e.g., a female or a male human), mouse, rat, guinea pig, dog, cat, horse, cow, pig, or non-human primates, such as a brown bear, monkey, chimpanzee, baboon or rhesus, and the terms “subject” and “patient” are used interchangeably herein.

The peptides described herein are administered in the form of sprays, for example, intranasal sprays.

The peptides described herein are administered in the form of drops, for example, intranasal drops or oral drops.

The peptides described herein are administered in the form of injections, for example, intravenous, subcutaneous, intramuscular, or intradermal injections.

The peptides described herein are administered in the form of tablets, capsules, chewable capsules, time-release or sustained-release tablets and capsules, powders, granules, teas, drops, or syrups for oral administration.

The peptides described herein are administered in the form of sublingual and buccal drug forms.

The peptides described herein are administered in the form of patches for transdermal administration.

The present disclosure further provides kits that can simplify the administration of any agent described herein. An illustrative kit of the present disclosure comprises any composition described herein in unit dosage form. In one embodiment, the unit dosage form is a container, such as a pre-filled syringe, which is sterile, containing any agent described herein and a pharmaceutically acceptable carrier, diluent, excipient, or vehicle. The kit can further comprise a label or printed instructions instructing the use of any agent described herein. The kit may also include a lid speculum, topical anesthetic, and a cleaning agent for the administration location. The kit can also further comprise one or more additional agents described herein. In one embodiment, the kit comprises a container containing an effective amount of a composition of the present disclosure and an effective amount of another composition, such those described herein.

The present disclosure is further illustrated by the following non-limiting examples.

In embodiments, potential targets of EPSI are selected from: INSR, IGF1R, LEPR, IL-6 receptor, IL-11 receptor, IL-12 receptor, IL-27 receptor, TNFRSF1A, LIFR, OSMR, EPOR, EGFR, GHR, IFNGR, and IFNAR.

The list of receptors was compiled based on a previous screening experiment, and also includes targets promising from the point of view of the therapy of metabolic pathologies (type 2 diabetes and NASH/NAFLD) and capable of activating Stat3: receptors associated with gp130 (IL27, IL6, IL11, LIFR, etc.); GCGR; GLP-1R; GIP-R; amylin receptor; GPR120; FGFRfamily; FXR (farnesoid); PPARs; LEPR.

### EXAMPLES

### Example 1: In Vitro Screening for Novel Regulatory Peptides to Treat Metabolic Disorders

### 1.1. Study Objective

The study objective was to evaluate the effect of individual peptides from bovine milk hydrolysate on the expression levels of key metabolically related genes in primary mouse fibroblasts.

### 1.2. Materials and Methods

### 1.2.1. Source of Biologically Active Peptides

Peptide composition of bovine milk hydrolysate was established using HPLC-MS/MS approach. The list of peptides was annotated manually; the primary criterion for selecting peptides was their novelty. As a result of the annotation, we have chosen 16 peptides derived from milk proteins CasA, CasB, CasK, LacB, CASA2, GlyCam1, OSTP, and PIGR proteins. DLSKEPSISRE (SEQ ID NO: 1) was among the list of peptides originating from GlyCam1 (UniProtKB— P80195) protein.

### 1.2.2. Cell Culture

The study was carried out in primary mouse fibroblasts. Fibroblasts were isolated from skin tissues of twenty C57BL/6 mice in accordance with the standard protocol (Seluanov (2010). Establishing primary adult fibroblast cultures from rodents. JoVE. 44). Fibroblasts were cultured in a complete DMEM/F12 medium containing 15% FBS for two days, after which they were incubated with individual peptides for 24 hours. The dose of each peptide was 0.5 mg/ml. The experiment was carried out in 2 biological replicates.

### 1.2.3. RNA Isolation and RT-PCR Protocol

The isolation of total RNA from cells was performed using the “ExtractRNA” reagent (Evrogen, Russia) analog of Trizol, according to the manufacturer's protocol. The quality of the isolated total RNA was assessed by measuring the concentration of RNA and the ratio of optical absorption at wavelengths of 260 nm and 280 nm.

The synthesis of the first strand of cDNA was performed using a commercially available MMLVRT Kit (Evrogen, Russia) according to the manufacturer's protocol.

For the screening, we have chosen genes playing the key role in the regulation of metabolically related intracellular processes:
1) Genes of mTOR signaling pathways: TSC1, TSC2, pS6K1;
2) Genes of insulin receptor signaling pathways: pAKT, IRS2; and
3) Genes for common transcriptional regulators of carbohydrate and lipid metabolism: Stat-3—a general regulator of metabolically-relevant intracellular processes; SREBP-1—a transcriptional regulator of genes responsible for lipid metabolism.

Design of the primers for the analysis of expression levels of studied genes was performed using Primer-BLAST primer designing tool (NCBI-NIH). The sequences of the primers used:

TSC1
(Fw-
(SEQ ID NO: 4)
TTATCCATCCTCTCGCTGCT,
Rv-
(SEQ ID NO: 5)
AGGTGCTGCTTCCCTGACT),
TSC2
(Fw-
(SEQ ID NO: 6)
ATGGATGTTGGCTTGTCCTC,
Rv-
(SEQ ID NO: 7)
TAAGCAGTTGTAGCAGACCA),
pS6K1
(Fw-
(SEQ ID NO: 8)
GACATGGCAGGAGTGTTTGA,
Rv-
(SEQ ID NO: 9)
TTTCCATAGCCCCCTTTACC,
pAKT
(Fw-
(SEQ ID NO: 10)
TCTATGGTGCGGAGATTGTG,
Rv-
(SEQ ID NO: 11)
CTTGATGTGCCCGTCCTTGT),
IRS2
(Fw-
(SEQ ID NO: 12)
CGGCCTCAACTATATCGCCA,
Rv-
(SEQ ID NO: 13)
GCGCTTCACTCTTTCACGAC),
Stat3
(Fw-
(SEQ ID NO: 14)
CTTGTCTACCTCTACCCCGACAT,
Rv-
(SEQ ID NO: 15)
GATCCATGTCAAACGTGAGCG),
SREBP-1
(Fw-
(SEQ ID NO: 16)
ATCGCAAACAAGCTGACCTG,
Rv-
(SEQ ID NO: 17)
AGATCCAGGTTTGAGGTGGG,
RPL27
(Fw-
(SEQ ID NO: 18)
AAGCCGTCATCGTGAAGAACA,
Rv-
(SEQ ID NO: 19)
CTTGATCTTGGATCGCTTGGC).

Real-time PCR was performed using commercially available kit qPCR mix-HS-SYBR+Low Rox (Evrogen, Russia) and 7500 real-time PCR System (Applied Biosystems). The results were normalized using primers for the housekeeping gene RPL27. We analyzed expression levels of TSC1, TSC2, pS6K1, pAKT, IRS2, SREBP-1, Stat3 genes. The amplification quality control was performed by recording the melting curves of the amplification products.

### 1.3. Results and Discussion

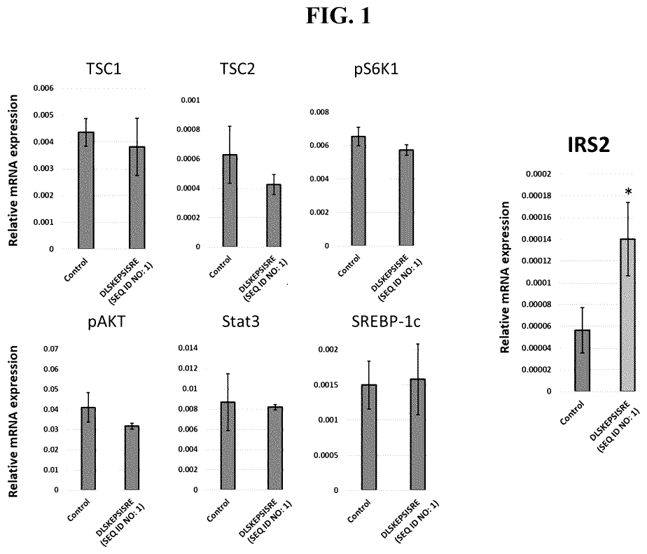

Of all the peptides screened, only DLSKEPSISRE (SEQ ID NO: 1) showed significant activity, which resulted in the change of IRS2 gene expression level. The results of the assessment of the activity of DLSKEPSISRE (SEQ ID NO: 1) peptide are represented on the FIG. 1. Incubation of fibroblasts with DLSKEPSISRE (SEQ ID NO: 1) (GlyCam1) peptide led to a significant IRS2 transcription induction. IRS2 is one of the main components of the insulin receptor intracellular signaling pathway (Kubota (2017). Imbalanced insulin actions in obesity and type 2 diabetes: key mouse models of insulin signaling pathway. Cell metab. 25(4): 797-810). Such type of effect is typical for the main anti-diabetic drugs including metformin (Ismail (2015). Molecular and immunohistochemical effects of metformin in a rat model of type 2 diabetes mellitus. Experimental and therapeutic medicine, 9(5), 1921-1930.) and GLP-1R agonists (Park (2006). Exendin-4 uses Irs2 signaling to mediate pancreatic β cell growth and function. Journal of Biological Chemistry, 281(2), 1159-1168) and show the ability of DLSKEPSISRE (SEQ ID NO: 1) peptide to influence the cellular response on glucose and insulin. Other chosen peptides demonstrated no effects on the expression levels of the studied genes.

### 1.4. Illustrative Conclusion

Peptide DLSKEPSISRE (SEQ ID NO: 1) (GlyCam1 fragment) affected the insulin signaling pathway in primary murine fibroblasts.

### Example 2. Evaluation of DLSKEPSISRE (SEQ ID NO: 1) Peptide Dose-Response Relationship in Mouse Fibroblasts

### 2.1. Study Objective

The study objective was to assess the optimal efficient dose for DLSKEPSISRE (SEQ ID NO: 1) in vitro.

### 2.2. Materials and Methods

The study was carried out in primary mouse fibroblasts. Isolation of fibroblasts was carried out with techniques described earlier (See Example 1). Fibroblasts were incubated with DLSKEPSISRE (SEQ ID NO: 1) peptide at different concentrations (0.005 mg/ml; 0.05 mg/ml; 0.5 mg/ml; 2.5 mg/ml) for 24 hours. Each treatment group was represented in two biological replicates.

Isolation of mRNA, synthesis of the first strand of cDNA, and analysis of expression levels of RPL27 and IRS2 genes by real-time PCR were performed with techniques described earlier (See Example 1).

### 2.3. Results and Discussion

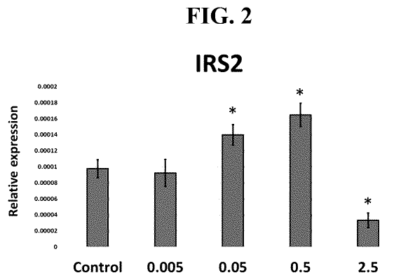

The results showing the relative expression levels of IRS2 gene in primary mouse fibroblasts after incubation with different doses of DLSKEPSISRE (SEQ ID NO: 1) peptide are shown in FIG. 2. We observed a stimulating effect of peptide DLSKEPSISRE (SEQ ID NO: 1) on IRS2 expression similar to those in the screening study. The effect was present at the peptide concentrations in the incubation medium of 0.05 mg/ml and 0.5 mg/ml, but not at 0.005 mg/ml. At 2.5 mg/ml DLSKEPSISRE (SEQ ID NO: 1) showed inhibition of IRS2 transcription. Though, the confluence of the cells was identical to the rest of the groups, meaning the absence of cytotoxic effect at the highest dose.

### 2.4. Illustrative Conclusion

DLSKEPSISRE (SEQ ID NO: 1) peptide (GlyCam1 fragment) showed a stimulatory effect on IRS2 expression levels in primary murine fibroblasts in a dose of 0.05 and 0.5 mg/ml. For the following studies, we have chosen a 0.05 mg/ml dose of DLSKEPSISRE (SEQ ID NO: 1), as more physiologically relevant.

### Example 3. The Effects of DLSKEPSISRE (SEQ ID NO: 1) Peptide in the In Vitro Models of Stress

### 3.1. Study Objective

The study objective was to evaluate DLSKEPSISRE (SEQ ID NO: 1) peptide effects on transcription regulation of metabolic-related genes in primary murine fibroblasts under stressful conditions.

### 3.2. Materials and Methods

The study was carried out in primary mouse fibroblasts. Isolation of fibroblasts was carried out with techniques described earlier (See Example 1). Fibroblasts were cultured for 3 days. The following stressors were applied separately: bacterial lipopolysaccharides (LPS), high glucose in the incubation medium, and serum-free incubation medium.

LPS is a major outer surface membrane component that induces an inflammatory response in cells. Fibroblasts were exposed to 0.5 μg/mL LPS for 24h, together with either DLSKEPSISRE (SEQ ID NO: 1) (0.05 mg/mL) or metformin (1 mM, medication to treat type II diabetes).

High glucose levels imitate conditions in diabetes. Fibroblasts were incubated with 5 mM glucose for 24 hours and then with 25 mM glucose for 24 hours together with either DLSKEPSISRE (SEQ ID NO: 1) (0.05 mg/mL) or metformin (1 mM).

Serum-free medium imitates cell starvation in the absence of growth factors. Cells were cultured on serum-free medium for 24 hours with added DLSKEPSISRE (SEQ ID NO: 1) (0.05 mg/mL) or metformin (1 mM).

Isolation of mRNA, synthesis of the first strand of cDNA, and RT-PCR were performed with techniques described earlier (See Example 1). Expression levels of IRS2, TNF-α, IL-6, and GSK3p (WNT-pathway) were analyzed.

Experimental groups (each group is represented in 3 biological replicates):
1) Control
2) Control+LPS
3) Control+Glucose
4) Control on serum-free medium

5)
DLSKEPSISRE (SEQ ID NO: 1)
6)
DLSKEPSISRE (SEQ ID NO: 1) + LPS
7)
DLSKEPSISRE (SEQ ID NO: 1) + Glucose
8)
DLSKEPSISRE
(SEQ ID NO: 1) on serum-free medium

9) Metformin
10) Metformin+LPS
11) Metformin+Glucose
12) Metformin on serum-free medium

### 3.3. Results and Discussion

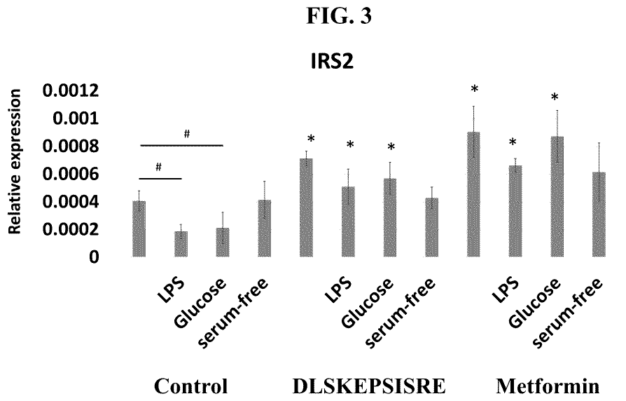

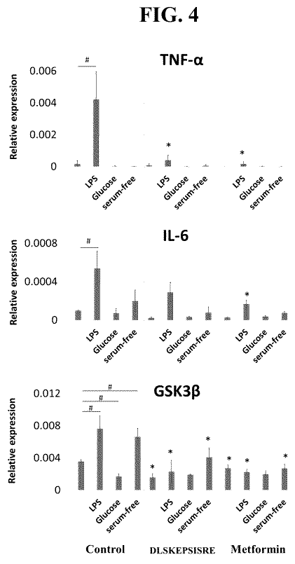

The effects of DLSKEPSISRE (SEQ ID NO: 1) peptide on transcription levels of studied genes under stressful conditions are shown in FIG. 3 and FIG. 4.

LPS significantly induced expression levels of pro-inflammatory cytokines TNF-α and IL-6 (FIG. 4) that indicate successful initiation of inflammatory response. Neither high glucose medium nor serum-free medium was able to change expression levels of pro-inflammatory cytokines (FIG. 4). Induction of GSK3p under starvation and inflammatory stress could be associated with impairments in insulin signaling (FIG. 4), and this effect is similar to that observed in different tissues in diabetic subjects (Takahashi-Yanaga (2013) Activator or inhibitor?GSK-3 as a new drug target. Biochem. Pharmacol. 86(2): 191-9). In the case of LPS treatment, this effect could be associated with a reduction in IRS2 expression (FIG. 3) which acts as a key participant of the insulin receptor signaling pathway. High glucose levels in the medium could be also associated with impairment of insulin receptor signaling that supposedly could be resulted in decreased IRS2 expression (FIG. 3).

DLSKEPSISRE (SEQ ID NO: 1) peptide upregulated IRS2 gene expression levels under normal conditions and stress (inflammation induction and high-glucose medium). The same effect was observed in metformin-treated groups (FIG. 3). Induction of IRS2 expression level is typical for the key anti-diabetic drugs such as metformin (Ismail (2015). Molecular and immunohistochemical effects of metformin in a rat model of type 2 diabetes mellitus. Experimental and therapeutic medicine, 9(5), 1921-1930.) and GLP-1R agonists (Park (2006). Exendin-4 uses Irs2 signaling to mediate pancreatic β cell growth and function. Journal of Biological Chemistry, 281(2), 1159-1168) and could serve as an indicator of activation of important metabolically-relevant intracellular pathways (Jhala (2003). cAMP promotes pancreatic β-cell survival via CREB-mediated induction of IRS2. Genes & development, 17(13), 1575-1580).

DLSKEPSISRE (SEQ ID NO: 1) peptide demonstrated a pronounced anti-inflammatory effect by reducing LPS-induced upregulation of expression of pro-inflammatory cytokines TNF-α and IL-6 (FIG. 3), similarly to metformin.

DLSKEPSISRE (SEQ ID NO: 1) peptide reduced GSK3p gene transcription levels under normal conditions and starvation and inflammatory stress. Increased GSK3p expression was previously described in diabetes (Takahashi-Yanaga (2013) Activator or inhibitor?GSK-3 as a new drug target. Biochem. Pharmacol. 86(2): 191-9), and metformin anti-diabetic action is partly associated with downregulation of GSK3p (Sarfstein (2013) Metformin downregulates the insulin/IGF-I signaling pathway and inhibits different uterine serous carcinoma (USC) cells proliferation and migration in p53-dependent or -independent manners. PloS one. 8(4): e61537.), and this mechanism is being considered a good therapeutic strategy that improves glucose metabolism in tissues. The increase in GSK3p expression during cell cultivation on the serum-free medium is associated with the inhibition of glycogen synthase, which under starvation conditions encourages the cell to use the reserves rather than synthesize glycogen. Inhibition of GSK3p strongly reduces the inflammatory response and the production of proinflammatory cytokines (Lappas (2014) GSK3p is increased in adipose tissue and skeletal muscle from women with gestational diabetes where it regulates the inflammatory response. PloS One, 9(12), e115854.).

### 3.4. Illustrative Conclusion

DLSKEPSISRE (SEQ ID NO: 1) peptide demonstrates beneficial hypoglycemic and anti-inflammatory effects in the in vitro models of stress.

### Example 4. Identification of DLSKEPSISRE (SEQ ID NO: 1) Peptide Pharmacophore

### 4.1. Study Objective

The study objective was to screen the functional activity of selected fragments of DLSKEPSISRE (SEQ ID NO: 1) peptide on mouse fibroblasts (expression level of IRS2) in comparison with the activity of the full-length peptide.

### 4.2. Materials and Methods

The study was carried out in primary mouse fibroblasts. Isolation of fibroblasts was carried out with techniques described earlier (See Example 1). Incubation of fibroblasts with individual peptides was carried out for 24 hours. All peptides were applied in concentration 100 μM which corresponds to DLSKEPSISRE (SEQ ID NO: 1) efficient dose of 0.05 mg/mL. Each treatment group was represented in two biological replicates.

The following DLSKEPSISRE (SEQ ID NO: 1) pharmacophores were tested: DLSKEP (SEQ ID NO: 20), SISRE (SEQ ID NO: 21), SKEPSIS (SEQ ID NO: 2), DLSK, LSKE, SKEP, ISRE, PSIS, SISR, EPSI, KEPS.

Isolation of mRNA, synthesis of the first strand of cDNA, and RT-PCR were performed with techniques described earlier (See Example 1). Expression levels of IRS2 and RPL27 genes were analyzed.

### 4.3. Results and Discussion

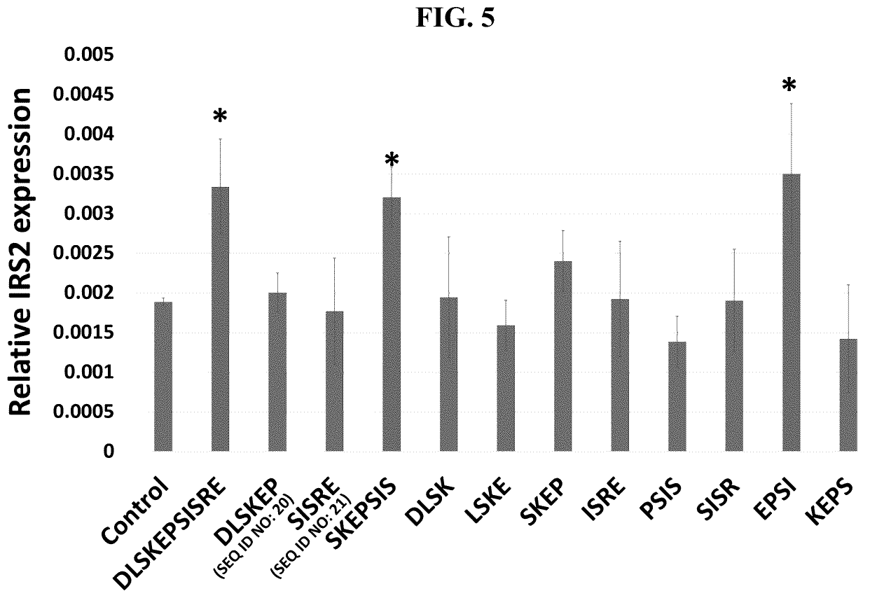

The effects of DLSKEPSISRE (SEQ ID NO: 1) peptide and its pharmacophores on transcription levels of IRS2 gene in primary mouse fibroblasts are represented in FIG. 5.

DLSKEPSISRE (SEQ ID NO: 1) peptide pharmacophores SKEPSIS (SEQ ID NO: 2) and EPSI induce IRS2 gene expression levels in mouse primary fibroblasts in the same manner as a full-size peptide. Other tested peptides did not show any significant activity.

### 4.4. Illustrative Conclusion

Peptides SKEPSIS (SEQ ID NO: 2) and EPSI demonstrated efficiency comparable to DLSKEPSISRE (SEQ ID NO: 1). The obtained data indicate EPSI as an active fragment of DLSKEPSISRE (SEQ ID NO: 1).

### Example 5. Testing of Anti-Inflammatory Activity of DLSKEPSISRE (SEQ ID NO: 1) Peptide Pharmacophores SKEPSIS (SEQ ID NO: 2) and EPSI

### 5.1. Study Objective

The study objective was to test the anti-inflammatory activity of SKEPSIS (SEQ ID NO: 2) and EPSI peptides in primary mouse fibroblasts.

### 5.2. Materials and Methods

The study was carried out in primary mouse fibroblasts. Isolation of fibroblasts was carried out with techniques described earlier (See Example 1). Incubation of fibroblasts with 100 μM individual peptides and 0.5 μg/mL LPS was carried out for 24 hours. Each treatment group was represented in two biological replicates.

Isolation of mRNA, synthesis of the first strand of cDNA, and RT-PCR were performed with techniques described earlier (See Example 1). Expression levels of RPL27, TNF-α, IL-6, and IRS2 genes were analyzed.

### 5.3. Results and Discussion

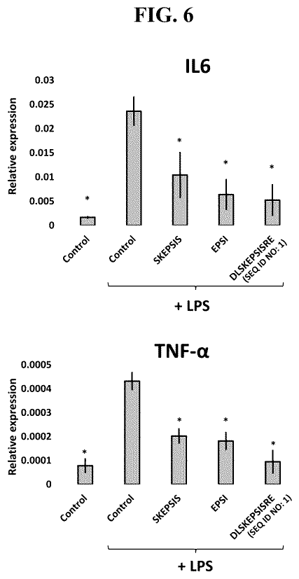

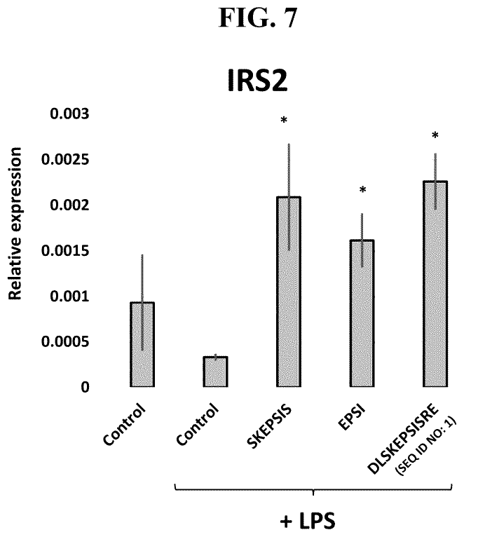

The influence of DLSKEPSISRE (SEQ ID NO: 1) pharmacophores SKEPSIS (SEQ ID NO: 2) and EPSI on the expression levels of pro-inflammatory cytokines and IRS2 genes during LPS-induced inflammatory response are represented on FIG. 6 and FIG. 7 respectively.

LPS significantly induced expression levels of TNF-α and IL-6 pro-inflammatory cytokines in mouse fibroblasts that indicate efficient activation of inflammatory response.

SKEPSIS (SEQ ID NO: 2) and EPSI performed significant anti-inflammatory effects comparable with DLSKEPSISRE (SEQ ID NO: 1) activity, by reducing LPS-evoked levels of TNF-α and IL-6 pro-inflammatory cytokines.

Both SKEPSIS (SEQ ID NO: 2) and EPSI induced IRS2 expression despite LPS-induced inflammatory response.

### 5.4. Illustrative Conclusion

EPSI and SKEPSIS (SEQ ID NO: 2) perform significant anti-inflammatory effects comparable with DLSKEPSISRE (SEQ ID NO: 1) activity.

### Example 6. Identification of Intracellular Signaling Pathway Responsible for the Effects Induced by EPSI Peptide

### 6.1. Study Objective

The objective of the study was to estimate the ability of EPSI peptide to influence the activity of selected signaling pathways.

### 6.2. Materials and Methods

### 6.2.2. Potential Targets Clusterization and Selection of Appropriate Reporter Systems

All studied receptors (potential targets) were clustered by the types of their signaling pathways and eventually by the transcription factors which act as the effectors of these pathways in the nucleus. An additional advantage of such type of clusterization is connected with the fact that the tested pathways are common and identification of the pathway could help in further identification of the target, even it is not mentioned in our list.

### Cluster 1: MAPK Pathway

Canonical MAPK pathway—one of the key activators of IRS2 expression (Udelhoven (2009). Identification of a region in the human IRS2 promoter essential for stress-induced transcription depending on SP1, NF1 binding, and ERK activation in HepG2 cells. Journal of molecular endocrinology, 44(2), 99-113).

c-Fos/c-Jun (AP1)—transcription factors described as the key effectors of the pathway. gp130—associated receptors are also able to induce this signaling pathway, but it should be accompanied by Stat activation (in case of the absence of Stat activation, receptors of the gp130 family should be excluded from the consideration). NFATc1—activates IRS2 expression, including signals from the insulin receptor. IRS2 promoter contains direct NFATc1 binding sites (Demozay (2011). Specific glucose-induced control of insulin receptor substrate-2 expression is mediated via Ca2+-dependent calcineurin/NFAT signaling in primary pancreatic islet p-cells. Diabetes, 60(11), 2892-2902).

INSR—canonic MAPK pathway; IGF1R—canonic MAPK pathway; EGFR—canonic MAPK pathway; TNFRSF1A—p38/MAPK (desired effect—inhibition of the receptor and p38/MAPK pathway activity that should be resulted in pERK-1/2 and the respective transcription factors activation).

Luciferase reporters used in the study:

NFATc1-reporter—effector of p38/MAPK pathway (Clipstone (1992). Identification of calcineurin as a key signaling enzyme in T-lymphocyte activation. Nature, 357(6380), 695-697).

AP1-reporter (c-Jun/c-Fos heterodimer)—effector of canonic MAPK+JNK pathway (Vasanwala (2002). Repression of AP-1 function: a mechanism for the regulation of Blimp-1 expression and B lymphocyte differentiation by the B cell lymphoma-6 protooncogene. The Journal of Immunology, 169(4), 1922-1929).

Cluster 2: key activators of pStat3 and pStat1 in different combinations

Stat3 is known to perform direct binding with IRS2 promoter that is shown in CHIP assay (Awazawa (2011). Adiponectin enhances insulin sensitivity by increasing hepatic IRS-2 expression via a macrophage-derived IL-6-dependent pathway. Cell metabolism, 13(4), 401-412).

IL6—Stat1/Stat3 (and any of homodimers); IL11—Stat1/Stat3 (and any of homodimers); IL27—Stat1/Stat3 (and any of homodimers); LIFR—Stat3/Stat3; IFNGR—Stat1/Stat1; IFNAR—Stat1/Stat2 (and any of homodimers)

Luciferase reporters used in the study:
M67 luciferase reporter. Could be activated by Stat1:Stat1; Stat1:Stat3; and Stat3:Stat3 (Besser (1999). A single amino acid substitution in the v-Eyk intracellular domain results in activation of Stat3 and enhances cellular transformation. Molecular and cellular biology, 19(2), 1401-1409).

The use of Stat1 inhibitor is required to distinguish the effects of Stat1 and Stat3. Pravastatin (Sigma Aldrich, USA) served as an appropriate inhibitor (Miklossy (2013). Therapeutic modulators of STAT signaling for human diseases. Nature reviews Drug discovery, 12(8), 611-629).
Cluster 3: pStat5
OSMR—Stat1, 3, 5; EPOR—Stat5; GHR—Stat1, 3, 5

Luciferase reporters used in the study:
Stat5-reporter: was assembled based on the described NFkB-reporter (Mitkin (2015). p53-dependent expression of CXCR5 chemokine receptor in MCF-7 breast cancer cells. Scientific reports, 5(1), 1-9) by cloning of five consensus Stat5-binding sites (sequence: GTTCTGAGAAAAGTAGTTCTGAGAAAAGTAGTTCTGAGAAAAGTAGTTCTGAGAAAAGTAGTTCTGAGAA AAGT (SEQ ID NO: 22)) using restriction sites XhoI and HindIII.
Cluster 4: pStat4
IL12—Stat4/Stat4

Luciferase reporters used in the study:

IFN-γ promoter (Gonsky (2000). Mucosa-specific targets for regulation of IFN-γ expression: lamina propria T cells use different cis-elements than peripheral blood T cells to regulate transactivation of IFN-γ expression. The Journal of Immunology, 164(3), 1399-1407).

### 6.2.3. The Cell Line Used in the Study

HepG2 human hepatocyte carcinoma cell line (HB-8065, ATCC, USA).

HepG2 cells were cultured in a complete DMEM medium containing 15% FBS.

Receptors INSR, IGF1R, IL-6r, IL-11r, IL-27r, TNFRSF1A, LIFR, OSMR, EPOR, EGFR, IFNGR, IL12RB, and IFNAR are highly expressed in HepG2 cells according to RNAseq data represented in Broad Institute Cancer Cell Line Encyclopedia (Barretina (2012). The Cancer Cell Line Encyclopedia enables predictive modeling of anticancer drug sensitivity. Nature, 483(7391), 603-607).

GHR is also highly expressed in HepG2 (Kim (2012). Orphan nuclear receptor small heterodimer partner negatively regulates growth hormone-mediated induction of hepatic gluconeogenesis through inhibition of signal transducer and activator of transcription 5 (STAT5) transactivation. Journal of Biological Chemistry, 287(44), 37098-37108).

### 6.2.4. Experimental Groups

HepG2+NFATc1-reporter
HepG2+NFATc1-reporter+EPSI
HepG2+NFATc1-reporter+TNFa (30 ng/ml)
HepG2+AP1-reporter
HepG2+AP1-reporter+EPSI
HepG2+AP1-reporter+insulin (100 nM)
HepG2+M67-reporter
HepG2+M67-reporter+EPSI
HepG2+M67-reporter+IL6 (50 ng/ml)
HepG2+M67-reporter+EPSI+Pravastatin (10 μM)
HepG2+Stat5-reporter
HepG2+Stat5-reporter+EPSI
HepG2+Stat5-reporter+erythropoietin (10 U/ml)
HepG2+Promoter IFN-γ
HepG2+Promoter IFN-γ+EPSI
HepG2+Promoter IFN-γ+IL12 (5 ng/ml)

Each group was represented in 3 biological replicates.

### 6.2.5. Experimental Procedures

HepG2 cells were electroporated with 10 μg of purified plasmid DNA and 0.1 μg of pRL-CMV Renilla luciferase control reporter vector (Promega, Madison, WI, USA) using the Neon Transfection System (Thermo Fisher Scientific, Waltham, MA, USA) and the following regimens: three 20 ms 1230 V pulses.

We performed incubation of transfected HepG2 cells with EPSI peptide (0.05 mg/ml) or with control agonists for 16 hours.

Luciferase activity was measured in Luminometer 20/20n (TurnerBioSystems, Sunnwale, USA) using Dual-Luciferase Reporter Assay System (Promega, Madison, USA) following the manufacturer's protocol.

### 6.3. Results and Discussion

EPSI induced the activity of M67 reporter (Stat1/Stat3).

Signaling pathway screened: Stat1:Stat1; Stat1:Stat3; Stat3:Stat3

Control compounds: IL6, 50 ng/ml (Liu (2016). Interleukin-6-stimulated progranulin expression contributes to the malignancy of hepatocellular carcinoma cells by activating mTOR signaling. Scientific reports, 6(1), 1-14); Pravastatin (Stat1 inhibitor), 10 μM (Menter (2011). Differential effects of pravastatin and simvastatin on the growth of tumor cells from different organ sites. PloS one, 6(12), e28813)

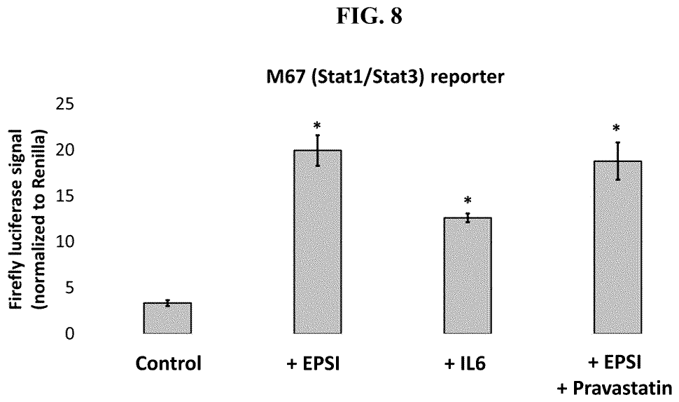

Incubation of HepG2 cells with EPSI peptide led to activation of the M67 reporter (FIG. 8), which indicates the ability of the peptide to induce Stat1/Stat3 activity and unambiguously indicates the corresponding group of receptors.

Application of pravastatin (Stat1 inhibitor) reduced the level of M67 reporter activation provided by the EPSI peptide, which indicates the ability of EPSI to activate at least Stat3. These data are in good agreement with the result of testing EPSI in vivo, where it was able to increase the level of pStat3 (tyr705) in mice hypothalamus as a result of intranasal administration (Example 10).

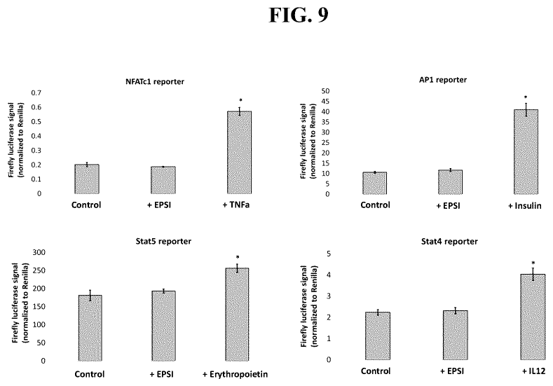

EPSI demonstrated no activity towards NFATc1-, AP1-, Stat5- or Stat4-reporters (FIG. 9).

### 6.4. Illustrative Conclusions

1) Reporter systems used in the study were successfully validated: all reporter systems demonstrated significant activation in HepG2 cells in response to the application of respective control agonists.
2) EPSI was not able to induce the activity of NFATc1-, AP1-, Stat5- and Stat4-reporters in HepG2 cells, which indicates EPSI does not activate the respective signaling pathways and does not act as an agonist of the studied receptors groups.
3) Incubation of HepG2 cells with EPSI peptide led to the activation of M67 reporter that indicates the ability of the peptide to induce Stat1/Stat3 activity and indicates the corresponding group of receptors.
4) Application of pravastatin (Stat1 inhibitor) did not decrease the level of M67 reporter activation provided by EPSI peptide that indicates the ability of EPSI to induce at least Stat3. These data are in good agreement with the result of testing EPSI in vivo, where it was able to increase the level of pStat3 (tyr705) in mice hypothalamus as a result of intranasal administration (Example 10).

### Example 7. Testing of the Influence of Antagonists of Specific Receptors on the Ability of EPSI Peptide to Induce M67 (Stat1/Stat3) Luciferase Reporter

### 7.1. Study Objective

The objective of the study was to assess the ability of EPSI peptide to activate M67 (Stat1/Stat3) luciferase reporter in HepG2 cells in the presence of antagonists of the specific receptors.

### 7.2. Materials and Methods

### 7.2.2. List of Receptor-Specific Antagonists Used in the Study

gp130 (IL27, IL6, IL11, LIFR). Inhibitor SC144 interrupts Stat3 phosphorylation by gp130 (Xu (2013). Discovery of a novel orally active small molecule gp130 inhibitor for the treatment of ovarian cancer. Molecular cancer therapeutics, 12(6), 937-949). gp130 Inhibitor: SC144 (Sigma-Aldrich, 5063870001).
GCGR. Potent and selective, non-competitive antagonist: L-168,049 (Tocris, Cat. No. 2311).
GLP-1R. Noncompetitive glucagon-like peptide-1 (GLP-1) receptor antagonist: VU 0650991 (Tocris, Cat. No. 6355).
GIP-R. [Pro3]-GIP (Human), (Phoenix Peptide, 027-51).
Amylin receptor. AC187 (Abcam, ab141150).
GPR120. AH 7614 (APExBIO Technology, Catalog No. B7792).
GPR119. GPR119 is a Gsa-protein associated receptor (Li (2018). GPR119 agonism increases glucagon secretion during insulin-induced hypoglycemia. Diabetes, 67(7), 1401-1413). Stat3 phosphorylation is performed in response to Gsa signaling (Ram (2001). G protein-coupled receptor signaling through the Src and Stat3 pathway: role in proliferation and transformation. Oncogene, 20(13), 1601-1606.). Selective Gsa antagonist: NF 449 (Tocris, Cat. No. 1391)
FGFR. Common inhibitor of all FGFR family receptors (FGFR1, 2, 3, 4): Erdafitinib (JNJ-42756493), (MedChemExpress USA, Cat. No.: HY-18708)
FXR (farnesoid). DY 268 (Tocris Cat. No. 5656)
PPAR-alpha: GW 6471 (Tocris, Cat. No. 4618)
PPAR-gamma: T0070907 (Selleck Chemicals, Catalog No. S2871)
PPAR-delta: GSK3787 (Selleck Chemicals, Catalog No. S8025)
LEPR. Leptin qA Human (ProSpec, CYT-353)

### 7.2.3. The Cell Line Used in the Study

HepG2 human hepatocyte carcinoma cell line (HB-8065, ATCC, USA).
HepG2 cells were cultured in a complete DMEM medium containing 15% FBS.

### 7.2.4. Experimental Groups

HepG2+M67-reporter
HepG2+M67-reporter+EPSI
HepG2+M67-reporter+EPSI+gp130
HepG2+M67-reporter+EPSI+L-168,049
HepG2+M67-reporter+EPSI+VU 0650991
HepG2+M67-reporter+EPSI+L-168,049+VU 0650991+[Pro3]-GIP
HepG2+M67-reporter+EPSI+AC187
HepG2+M67-reporter+EPSI+AH 7614
HepG2+M67-reporter+EPSI+NF 449
HepG2+M67-reporter+EPSI+Erdafitinib
HepG2+M67-reporter+EPSI+DY 268
HepG2+M67-reporter+EPSI+GW 6471+T0070907+GSK3787
HepG2+M67-reporter+EPSI+Leptin qA

Each group was represented in 3 biological replicates.

### 7.2.5. Experimental Procedures

HepG2 cells were electroporated with 10 μg of purified plasmid DNA of M67 reporter and 0.1 μg of pRL-CMV Renilla luciferase control reporter vector (Promega, Madison, WI, USA) using the Neon Transfection System (Thermo Fisher Scientific, Waltham, MA, USA) and the following regimens: three 20 ms 1230 V pulses.

Transfected HepG2 cells were co-incubated with EPSI peptide (0.05 mg/mL) and antagonists of the selected receptors.

Detection of luciferase signals was performed 16 hours after the compounds' application. Luciferase activity was measured in Luminometer 20/20n (TurnerBioSystems, Sunnwale, USA) using Dual-Luciferase Reporter Assay System (Promega, Madison, USA) following the manufacturer's protocol.

### 7.3. Results and Discussion

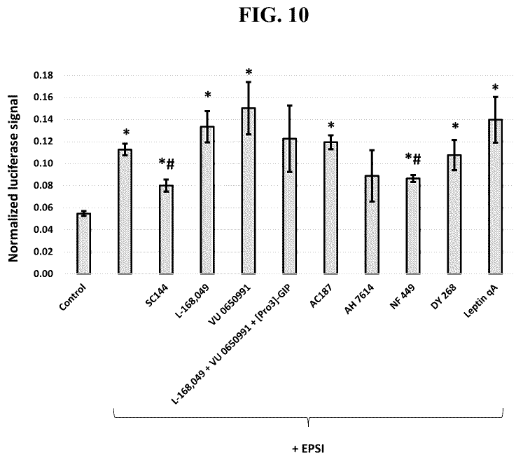

Incubation of HepG2 cells with EPSI peptide led to activation of Stat3-dependent M67 luciferase reporter (FIG. 10) that correlates with previous data (Example 6) and with phosphorylation of Stat3 (Tyr705) in the hypothalamus of experimental mice in vivo (Example 10).

gp130 (SC144) and Gsa (NF 449) antagonists partially inversed the activating effect of EPSI peptide that indicates these classes of targets as potential effectors of the peptide action.

In the case of gp130-associated targets, the most interesting effect should relate to their hypothalamic action: activation of these receptors has resulted in intensified Stat3-signaling that induces leptin-dependent pathways and alleviate leptin resistance conditions. Therefore, agonists of the receptors of gp130 family normalize eating behavior and are considered as anti-obesogenic agents (Cron (2016). The role of gp130 receptor cytokines in the regulation of metabolic homeostasis. Journal of Experimental Biology, 219(2), 259-265). Mechanistically, activation of the receptors of gp130 family leads to induction of IRS2 expression (Awazawa (2011). Adiponectin enhances insulin sensitivity by increasing hepatic IRS-2 expression via a macrophage-derived IL-6-dependent pathway. Cell metabolism, 13(4), 401-412) and phosphorylation of Akt (Steinberg (2009). Ciliary neurotrophic factor stimulates muscle glucose uptake by a PI3-kinase-dependent pathway that is impaired with obesity. Diabetes, 58(4), 829-839).

Gsa protein serves as an effector of the range of GPCRs considered as the targets for therapy of metabolic disorders: GPR119, GIP-R, GLP1R, GCGR, MC4R, etc. Gsa is known to promote the effect of incretins by inducing glucose-stimulated insulin secretion in the pancreas and production of incretins themselves by intestinal and pancreatic cells (Zhu (2013). GPR119 agonists: a novel strategy for type 2 diabetes treatment. Diabetes mellitus-insights and perspectives). In adipose tissue Gsa-mediated activation of cAMP is associated with induced lipolysis that could play an important role in obesity treatment (Guilherme (2008). Adipocyte dysfunctions linking obesity to insulin resistance and type 2 diabetes. Nature reviews Molecular cell biology, 9(5), 367-377). In muscle tissue normalization of Gsa-cAMP signaling disturbed during metabolic diseases results in improved insulin sensitivity and glucose homeostasis, reduction of lipid accumulation, induction of fatty acid oxidation, and energy expenditure (Yang (2016) Targeting cAMP/PKA pathway for glycemic control and type 2 diabetes therapy. Journal of molecular endocrinology, 57(2), R93-R108). Gsa is known to be involved in the induction of IRS2 expression (Portha (2011) Activation of the GLP-1 receptor signaling pathway: a relevant strategy to repair a deficient beta-cell mass. Experimental diabetes research, 2011) that may serve as one of the mechanistic explanations of its action on insulin sensitivity. Gsa-cAMP signaling in hypothalamus is involved in food intake regulation that results in alleviation of obesity and insulin resistance conditions (Yang (2016). Targeting cAMP/PKA pathway for glycemic control and type 2 diabetes therapy. Journal of molecular endocrinology, 57(2), R93-R108). This axis underlies central effects of main metabolic regulators with high therapeutic potential such as incretins and melanocortin (Chen (2009). Central nervous system imprinting of the G protein Gsa and its role in metabolic regulation. Cell metabolism, 9(6), 548-555).

Incubation of HepG2 cells with Erdafitinib (which was used in the study as an FGFR antagonist) led to total cell death. This effect of Erdafitinib should be considered as a result of its action as a chemotherapeutic agent and did not allow us to estimate the role of FGFRs in EPSI-induced effects.

Incubation of HepG2 cells with three PPAR antagonists (GW 6471, T0070907, and GSK3787) led to significant induction of Stat3 reporter activity. This effect comes to an agreement with the published data (Bright (2008). Targeting PPAR as a therapy to treat multiple sclerosis. Expert opinion on therapeutic targets, 12(12), 1565-1575.) but does not allow us to estimate whether the effect of EPSI is triggered by PPARs.

### 7.4. Illustrative Conclusion

The obtained results indicate the classes of receptors associated with gp130 and Gsa protein, as potential targets of EPSI peptide.

### Example 8. Identification of EPSI Peptide Targets in a Comprehensive Functional GPCR Panel

### 8.1. Study Objective

The objective of the study was to perform the high-throughput screening of EPSI peptide action on 163 different GPCRs.

### 8.2. Materials and Methods

EPSI peptide was tested in the dose of 100 μM in 326 functional assays (163 GPCRs both in agonist and antagonist modes) combined in Full Functional GPCR Panel (item P343) provided by Eurofins Cerep SA.

Each cell-based assay was performed by application of the peptide to cell lines expressing individual receptors with subsequent measuring Ca2+, IP1, or cAMP depending on the receptor type. In the case of antagonist mode assays, the peptide was applied together with the control agonist.

Cellular agonist effect was calculated as a % of control response to a known reference agonist for each target and cellular antagonist effect was calculated as a % inhibition of control reference agonist response for each target.

### 8.3. Results and Discussion

EPSI peptide induced activating effect provided by 10 nM of control agonist NPS (neuropeptide S) by 56.4±8.5% in HEK-293 cells expressing NPSR1 (the receptor of neuropeptide S). Activation of NPSR1 was measured by the changes in intracellular Ca2+ levels. This action of EPSI peptide could characterize its profile as a strong positive allosteric modulator (PAM) of NPSR1 since it demonstrated no effect in an agonist mode.

Neuropeptide S is considered as a potent regulator of appetite and eating behavior (Botticelli (2021). The Neural Network of Neuropeptide S (NPS): Implications in food intake and gastrointestinal functions. Pharmaceuticals, 14(4), 293) and a stimulator of fatty acids oxidation in adipose tissue (Ahmad (2020). Neuropeptide S receptor gene Asn107 polymorphism in obese male individuals in Pakistan. PloS one, 15(12), e0243205). NPSR1 is a GPCR that acts through both Gaq and Gas. It is highly expressed in hypothalamus and can induce Stat3 signaling through Gas activation that could serve as a pathway mimicking leptin action (Cline (2007). Anorexigenic effects of central neuropeptide S involve the hypothalamus in chicks (Gallus gallus). Comparative Biochemistry and Physiology Part A: Molecular & Integrative Physiology, 148(3), 657-663).

EPSI peptide acted as an antagonist of GPR109A receptor reducing by 37.1±2.1% the signal evoked by 300 nM of nicotinic acid in RBL cells expressing GPR109A.

EPSI peptide acted as antagonist of FFAR2 (GPR43) receptor reducing by 28.0±1.5% the signal induced by 1 mM of sodium acetate in HEK-293 cells expressing FFAR2.

Mechanistically, inhibition of both GPR109A and FFAR2 should be associated with reduced Gαi signaling that results in induction of Gas signaling and elevated cAMP levels in different tissues (Priyadarshini (2018). Role of short-chain fatty acid receptors in intestinal physiology and pathophysiology. Comprehensive Physiology, 8(3), 109.) typical for the action of agonists of metabolically relevant receptors (e.g., without limitation, GLP1R, GIP-R, AMY2, MC4R). FFAR2 inhibition is considered as a promising strategy of type 2 diabetes treatment (Tang (2015). Loss of FFA2 and FFA3 increases insulin secretion and improves glucose tolerance in type 2 diabetes. Nature medicine, 21(2), 173-177): FFAR2 is highly expressed in the pancreas, and its activation is associated with reduced insulin secretion that in the long term leads to insulin resistance progression (Priyadarshini (2015). An acetate-specific GPCR, FFAR2, regulates insulin secretion. Molecular endocrinology, 29(7), 1055-1066). Inhibition of GPR109A, which is primarily expressed in adipocytes and hepatocytes, results in alleviation of insulin resistance conditions (Heemskerk (2014). Long-term niacin treatment induces insulin resistance and adrenergic responsiveness in adipocytes by adaptive downregulation of phosphodiesterase 3B. American Journal of Physiology-Endocrinology and Metabolism, 306(7), E808-E813) and induction of lipolysis (Geisler (2021). The Role of GPR109a Signaling in Niacin Induced Effects on Fed and Fasted Hepatic Metabolism. International journal of molecular sciences, 22(8), 4001).

EPSI peptide induced activating effect provided by 30 nM of control agonist Lysophosphatidic acid by 29.2±3.0% in CHO cells expressing LPAR3. This action of EPSI peptide could characterize its profile as a positive allosteric modulator (PAM) of LPAR3 since it demonstrated no effect in an agonist mode.

EPSI peptide reduced cAMP levels in CHO cells expressing M2 muscarinic receptor demonstrating the efficiency of 26.8±6.8% compared to the control agonist 3 μM acetylcholine (which acts oppositely by inducing cAMP levels). In antagonist mode EPSI demonstrated no effects on acetylcholine-induced activation of M2 receptor that indicates EPSI as a potential inverse agonist of M2 receptor.

### 8.4. Illustrative Conclusion

EPSI peptide acted as a positive allosteric modulator of NPSR1 and LPAR3 receptors, as an antagonist of GPR109A and FFAR2 receptors, and as an inverse agonist of M2 muscarinic receptor.

### Example 9. Dose-Dependent Action of EPSI as an Antagonist of FFAR2 (GPR43) Receptor

### 9.1. Study Objective

The objective of the study was to test the ability of EPSI peptide to provide antagonistic action on FFAR2 (GPR43) receptor in a dose-dependent manner

### 9.2. Materials and Methods

EPSI peptide was tested in the doses of 0.03, 0.16, 4, and 20 μM in FFAR2 cell-based functional antagonistic assay provided by Eurofins Cerep SA. The assay was performed in HEK-293 cell line expressing FFAR2 and stimulated with 1 mM sodium acetate (control reference agonist) for FFAR2 signaling induction. Fluorimetric measurement of intracellular Ca2+ levels was performed after the peptide application. Cellular antagonist effect was calculated as a % inhibition of control reference agonist response.

### 9.3. Results and Discussion

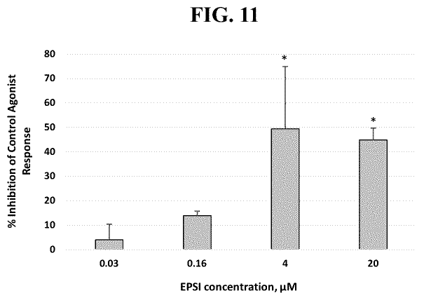

EPSI peptide acted as an antagonist of FFAR2 (GPR43) receptor reducing the signal induced by 1 mM of sodium acetate in HEK-293 cells expressing FFAR2 in a dose-dependent manner. 4 and 20 μM appeared to be effective doses leading to significant inhibition of FFAR2 activity, while in case of 0.03 and 0.16 μM the effects were insignificant (FIG. 11).

### 9.4. Illustrative Conclusion

EPSI acts as an antagonist of FFAR2 (GPR43) receptor in a dose-dependent manner.

### Example 10. Analysis of the Influence of EPSI Peptide on the Activity of Stat3 Signaling in the Mouse Brain

### 10.1. Study Objective

The objective of the study was to estimate the ability of EPSI peptide to induce STAT3 activity in the mouse hypothalamus.

### 10.2. Materials and Methods

### 10.2.1. Laboratory Animals

Fifteen C57Bl/6J 15 weeks old mice (5 animals per group) were used in the study.

### 10.2.2. Compounds

We performed food deprivation 24 hours prior to the administration of the compounds. The animals of the experimental group were administered with 10 mg/kg EPSI intranasally (EPSI was dissolved in 10 μL of saline per mouse, 5 μL in each nostril). Positive control animals were administered with 5 mg/mL leptin intraperitoneally (leptin was dissolved in 100 μL of saline per mouse). Negative control mice were administered with 100 μL of saline intraperitoneally. The mice were euthanized 60 minutes after the administration of the compounds.

### 10.2.3. Brain Fixation and Cryoprotection

Narcosis was performed using the combination of Zoletil (20 mg/kg) and Rometar (5 mg/kg). Next, transcardial perfusion was performed with saline to wash out the blood, then with a 10% formalin solution. After complete fixation, the animal bodies were decapitated, the brain was extracted and additionally fixed in 10% formalin, after which it was washed in PBS 3 times for 30 minutes and placed overnight in a 30% sucrose solution in PBS until the tissue was completely impregnated.

### 10.2.4. Histological Samples Preparation

The brain samples were placed in a mold, and poured with the freezing medium. Then the samples were frozen in nitrogen vapor and 10 micron thick slices were made using Thermo Scientific Microm HM525 cryostat. The slices were immediately mounted on slides. 4-5 glasses with 8 slices on each were obtained from each brain.

10.2.5. Immunohistochemistry staining

The slices were additionally fixed in 10% formalin for 10 minutes, after that, they were washed in PBS 5 times for 8 minutes. Then the preparations were incubated in a solution of hydrogen peroxide (1% H2O2 with 0.3% NaOH in PBS) for 20 minutes, in 0.3% glycine solution for 10 minutes, and in 0.03% SDS for 10 minutes. After that, the preparations were incubated for an hour in a blocking solution (PBS with 10% goat serum and 0.3% Triton-X-100). Then the slices were circled with a hydrophobic marker and applied with primary pSTAT3 (Y705) antibodies (Cell Signaling Technology) diluted in a block solution in a ratio of 1:300. Incubation was carried out overnight (16-18 hours) at +4° C. in a humid chamber. Then the preparations were washed 3 times in PBS and incubated with secondary AlexaFluor 488 antibodies diluted in a 1:600 block solution overnight (16-18 hours) at +4° C. in a humid chamber. After incubation, the preparations were washed 3 times in PBS and mounted in Vectashield mounting medium with DAPI under a cover glass.

### 10.2.6. Images Preparation and Statistic Analysis

The images were received using an inverted microscope Nikon Eclipse Ni. The number of positive cells was calculated using ImageJ software. The data are represented as the ratio of the number of pStat3-positive cells to the total number of nuclei (stained with DAPI).

The results were statistically processed in GraphPad Prism 8.0.1 software. A single-factor ANOVA test was used for analysis, paired comparisons were carried out based on the Dunnet test. The result was considered statistically significant at p≤0.05.

10.3. Results and discussion

Leptin is Known to Regulate Appetite, Thermogenesis, and Blood Glucose Level by Influencing the Activity of media-basal hypothalamic neurons. Activation of leptin receptor (LEPR) in hypothalamic cells is associated with the induction of a series of signaling pathways. JAK-STAT signaling pathway is considered as the main LEPR effector and as the link involved in the regulation of eating behavior (Kwon (2016). Leptin signaling pathways in hypothalamic neurons. Cellular and Molecular Life Sciences, 73(7), 1457-1477). The level of phosphorylated Stat3 is considered as the main indicator of both LEPR activation and induction of the pathways responsible for appetite regulation (Ladyman (2013). JAK-STAT and feeding. Jak-stat, 2(2), e23675). During the current study, we investigated the ability of EPSI peptide to induce STAT3 signaling compared to leptin as the positive control.

For the pStat3-positive cells staining and visualization, we used previously published protocol (Roujeau (2014). New pharmacological perspectives for the leptin receptor in the treatment of obesity. Frontiers in endocrinology, 5, 167) with modifications.

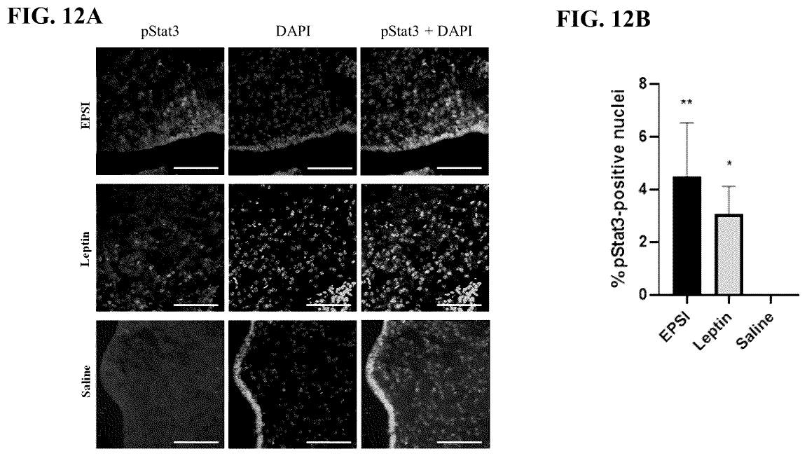

pStat3-positive cells were found in hypothalamic sections of both the mice treated with EPSI and Leptin (FIG. 12A). In hypothalamic sections of negative control mice received saline no pStat3-positive cells were observed.

Applying ImageJ software we quantified pStat3-positive cells and calculated the ratio of the number of pStat3-positive cells to the total number of nuclei (stained with DAPI).

Leptin induced pStat3 activation in 3% of the cells that correspond to previously published data (Roujeau (2014). New pharmacological perspectives for the leptin receptor in the treatment of obesity. Frontiers in endocrinology, 5, 167). Administration of EPSI peptide induced more intense translocation of Stat3 to the nucleus compared to leptin and resulted in 4.5% of pStat3-positive cells (FIG. 12B). These data indicate that EPSI is able to activate hypothalamic pathways responsible for appetite regulation at list in the same manner as leptin.

### 10.4. Illustrative Conclusion

The efficiency of hypothalamic pStat3 activation by EPSI peptide is comparable to leptin which indicates EPSI is a regulator of eating behavior.

### Example 11. Study on the Effect of Acute Administration of EPSI Peptide on the Appetite of Mice

### 11.1. Study Objective

The study objective was to evaluate the effects of intranasal EPSI administration on appetite in the test for food consumption in a home cage. In addition, to compare the observed effects with the comparison drug leptin.

### 11.2. Materials and Methods

### 11.2.1. Animals and Treatment

The work was performed on 30 mature male mice of the first generation of CBA and C57Bl/6 hybrids weighing 22-24 grams. The animals were divided into treatment groups (Table 1).

Solutions for intranasal (i.n.) administration were prepared by dissolving the peptide in saline. The volume of i.n. administration: 1 μl per 1 g of animal weight. Solutions for intraperitoneal (i.p.) administration were prepared by dissolving the drug in saline. The volume of i.v. administration: 10 μl per 1 g of animal weight. Solutions were prepared directly on the day of the experiment before the experiment.

TABLE 1
Experimental groups

| | Number of | |
|---|---|---|
| Group Name | Animals | Tested Substances |
| 1. Control | 10 | i.n. and i.p. administration of saline prior to |
| | | testing |
| 2. EPSI | 10 | i.n. administration of a 5 mg/kg EPSI |
| | | peptide; i.p., administration of saline prior to |
| | | testing |
| 3. Comparison | 10 | i.p. administration of a 1 mg/kg leptin; i.n. |
| Drug Leptin | | administration of saline prior to testing |

### 11.2.2. Experimental Design

All manipulations with animals were performed at the end of the adaptation period of 14 days.

Day 1—Adaptation of animals to the home cage food consumption test. In the evening, animals were deprived of food for 12 hours (overnight).

Day 2—Administration of the test substances and the food consumption test in a home cage for 1.5 hours. Before and after the test, the animals were weighed.

### 11.2.3. Behavioral Test for Appetite: Food Consumption in a Home Cage

This test was performed to assess the appetite of animals after the treatment with tested compounds. The test was performed in 2 days.

Day 1—The day before the test, the animals were adapted to the experimental conditions. For this, a cage similar to the home cage was used, in the center of which an empty Petri dish was placed. The adaptation lasted 90 minutes. In the evening, animals were deprived of food overnight.

Day 2—The test was performed in a cage used for adaptation, in the center of which two pre-weighed food pellets were placed in a Petri dish. After 30, 60, and 90 minutes, the food from the cage was weighed.

The use of a home cage (as well as preliminary adaptation a day before testing) as a stage for the test allowed us to evaluate the food motivation of the animals (appetite, the severity of hunger) under standard conditions, without affecting the anxiety component characteristic of the new conditions (Anahita et al., 1997 with changes).

### 11.2.4. Statistical Analysis

Statistical data processing was carried out using nonparametric criteria (Mann-Whitney) for samples with a distribution other than normal. Normally distributed data were analyzed with one-way ANOVA (only Treatment factor) or repeated measures ANOVA (dynamics) with post hoc Fisher test. The data are presented as mean±standard error of the mean (SEM). Differences between groups were considered significant at p<0.05.

### 11.3. Results

### 11.3.1. Food Consumption in a Home Cage

After overnight fasting and before the test, animals from experimental groups were weighed. No differences in body weight between the experimental groups were found. The average mouse weight was 24.93±0.494 g.

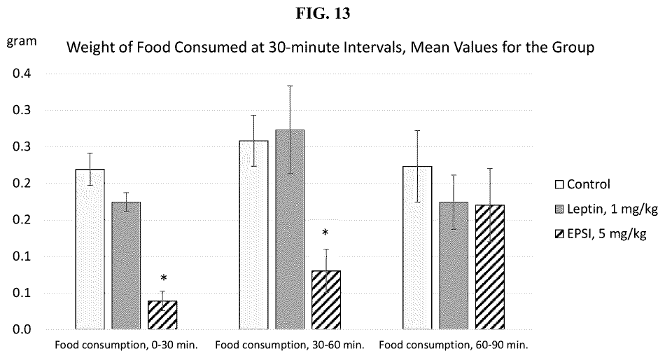

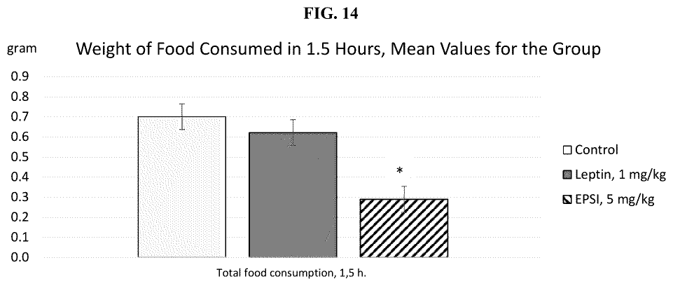

Mice treated with 1 mg/kg leptin did not differ from the control animals based on the amount of food consumed over 90 minutes of the experiment (FIGS. 13-14). At the same time, the peptide EPSI at 5 mg/kg had a pronounced anorexigenic effect, reducing food intake relative to the control group at the interval of 0-30 minutes (0.04±0.014 g and 0.22±0.022 g in the control group), 30-60 minutes (0.08±0.03 and 0.26±0.035 g), as well as total food consumed (0.29±0.066 and 0.7±0.064 g, respectively).

### 11.4. Discussion

The peptide EPSI when administered at a dose of 5 mg/kg, showed an anorexigenic effect in the home cage eating test, reducing food consumption during the first hour of observation

The effect of EPSI is similar to the effect of glucagon-like peptide-1 (GLP-1), which permanently reduces animal food intake, starting to act immediately after injection (Dalboge et al., 2020).

### 11.5. Illustrative Conclusions

Intranasal administration of EPSI at a dose of 5 mg/kg resulted in a pronounced reduction of food intake in non-stressful conditions of the home cage, which indicates an anorexigenic effect of the peptide.

### Example 12. The Study of the Effects of EPSI Peptide on the Severity of Symptoms of Metabolic Disorder in Laboratory Rats Caused by a High-Sucrose Diet

### 12.1. Study Objective

The study objective was to study the effect of intraperitoneal administration of EPSI peptide on the severity of symptoms of metabolic disorder in laboratory Sprague-Dawley rats on a high-sucrose diet (HSD).

The work was performed in 3 stages.

Stage 1. The study of the effect of i.p. administration of EPSI in the glucose tolerance test in animals with symptoms of metabolic disorder after HSD.

Stage 2. The study of the effect of i.p. administration of the EPSI peptide in animals with symptoms of metabolic disorder after HSD on the blood and liver biochemical parameters.

Stage 3. Analysis of EPSI peptide effect on phosphorylation levels of proteins participating in intracellular insulin signaling in the liver of animals with symptoms of metabolic disorder induced by HSD.

### 12.2. Materials and Methods

### 12.2.1. Experimental Design

12.2.1.2. Stage 1. The Study of the Effect of i.p. Administration of EPSI in the Glucose Tolerance Test in Animals with Symptoms of Metabolic Disorder Under HSD.

Fifty adult Sprague-Dawley male rats were used

An additional drinker with a 30% sucrose solution was placed in the cage with rats, starting from the first day, for at least five weeks (high sucrose diet, HSD).

From the 8th day, the weight of the animals was measured once every two days. Once a week (8, 15, 22, 28 days), the concentration of glucose in the blood of animals was measured using an ACCU-CHEK Active glucometer (Roche, IN, United States). The sucrose consumption was evaluated by weighing the drinker (once in 2 days, the average per cage). The development of the metabolic condition in rats was considered when a significant increase in blood glucose concentration for two consecutive measurements relative to the values in the control group was registered.

Animals with symptoms of a metabolic disorder induced by HSD were then tested for glucose tolerance. For this, the animals were deprived of food and drinkers with sucrose solution 12 hours before glucose administration. Peptide EPSI was introduced at the corresponding time points prior to glucose i.g. injection (Table 2). At the end of the test, the animals were returned to the cage with food and drinker with 30% sucrose solution.

The animals' blood glucose levels were measured at 0 (basal glucose level after food deprivation and before drug administration) and 15, 30, 60, 120 minutes after i.g. administration of glucose.

TABLE 2
Experimental groups (Stage 1)

| | Animal | |
|---|---|---|
| Group name | N | Test substance |
| 1. Control | 5 | Regular diet. I.p. saline administration right |
| | | before glucose administration EPSI |
| 2. Control HSD | 5 | HSD. I.p. saline administration right before |
| | | glucose administration |
| 3. EPSI, | 5 | HSD. I.p. administration of EPSI at a dose of |
| 1 mg/kg, 0 h. | | 1 mg/kg right before glucose administration |
| 4. EPSI, | 5 | HSD. I.p. administration of EPSI at a dose of |
| 1 mg/kg, 2 h. | | 1 mg/kg 2 hours before glucose |
| | | administration |
| 5. EPSI, | 5 | HSD. I.p. administration of EPSI at a dose of |
| 1 mg/kg, 12 h. | | 1 mg/kg 12 hours before glucose |
| | | administration |
| 6. EPSI, | 5 | HSD. I.p. administration of EPSI at a dose of |
| 1 mg/kg, 24 h. | | 1 mg/kg 24 hours before glucose |
| | | administration |
| 7. EPSI, | 5 | HSD. I.p. injection of EPSI at a dose of 10 |
| 10 mg/kg, 0 h. | | mg/kg right before glucose administration |
| 8. EPSI, | 5 | HSD. I.p. injection of EPSI at a dose of 10 |
| 10 mg/kg, 2 h. | | mg/kg 2 hours before glucose administration |
| 9. EPSI, | 5 | HSD. I.p. administration of EPSI at a dose of |
| 10 mg/kg, 12 h. | | 10 mg/kg 12 hours before glucose |
| | | administration |
| 10. EPSI, | 5 | HSD. I.p. administration of EPSI at a dose of |
| 10 mg/kg, 24 h. | | 10 mg/kg 24 hours before glucose |
| | | administration |

EPSI and glucose were administered in a volume of 1 μl per 1 g of weight at the appropriate concentrations.

12.2.1.3. Stage 2. The Study of the Effect of i.p. Administration of the EPSI Peptide in Animals with Symptoms of Metabolic Disorder after HSD on the Blood and Liver Biochemical Parameters.

Twenty-four adult Sprague-Dawley male rats were used from stage 2.

After stage 2, animals were kept on the HSD for another week. After that, rats were deprived of food and sucrose-containing drinking bottles for 12 hours. Peptide EPSI was introduced prior to blood collection at the corresponding time points (Table 3). Blood was collected after decapitation. Analysis of concentrations of glucose, triglycerides, cholesterol, low-density lipoprotein (LDL), high-density lipoprotein (HDL), and insulin was carried out in blood serum.

TABLE 3
Experimental groups (Stage 2)

| | Number of | |
|---|---|---|
| Group name | animals | Test substance |
| 1. Control | 2 | Regular diet. i.p., saline administration |
| | | according to the scheme of administration |
| | | of EPSI, blood collection 2 hours after |
| | | treatment |
| 2. Control HSD | 5+ | 6-weeks HSD, i.p. administration of saline |
| | | according to the peptide administration |
| | | scheme, blood collection 2 hours after |
| | | treatment |
| 3. EPSI, | 4 | 6-weeks HSD, i.p. peptide administration |
| 1 mg/kg, 2 h. | | at a dose of 1 mg/kg, blood collection 2 |
| | | hours after treatment |
| 4. EPSI, | 4 | 6-weeks HSD, i.p. peptide administration |
| 1 mg/kg, 12 h. | | at a dose of 1 mg/kg, blood collection 12 |
| | | hours after treatment |
| 5. EPSI, | 4 | 6-weeks HSD, i.p. peptide administration |
| 10 mg/kg, 2 h. | | at a dose of 10 mg/kg, blood collection 2 |
| | | hours after treatment |
| 6. EPSI, | 5 | 6-weeks HSD, i.p. peptide administration |
| 10 mg/kg, 12 h. | | at a dose of 10 mg/kg, blood collection 12 |
| | | hours after treatment |

EPSI was administered in a volume of 1 μl per 1 g of weight at the appropriate concentrations.

### 12.2.1.4. Stage 3. Analysis of the Phosphorylation Levels of AKT and 70S6k1 Proteins in the Liver of Experimental Animals.

Twenty-six rats were administered EPSI at a dose of 1 and 10 mg/kg 2 and 12 hours before euthanasia. Then these rats were subjected to injection anesthesia with a combination of Zoletil and Xylazine. On anesthesia, the animals were injected with insulin solution (10 U/kg body weight, volume 100 μl/kg body weight; Kanisulin, 40 U/ml, Intervet International Gmb, Germany) into the vena cava, and after minutes, liver samples were dissected (at least 100 mg of tissue was snap-frozen in liquid nitrogen, and then stored at −80). Then the animals were euthanized by an overdose of Zoletiliand Xylazine combination. 2 hours after

TABLE 4
Experimental groups (Stage 3)

| | Number of | |
|---|---|---|
| Group name | animals | Test substance |
| 1. Control | 3 | Regular diet. i.p., saline administration |
| | | according to the scheme of administration |
| | | of EPSI, insulin solution injection, liver |
| | | sample collection 2 hours after treatment |
| 2. Control HSD | 5 | 6-weeks HSD, i.p. administration of saline |
| | | according to the peptide administration |
| | | scheme, insulin solution injection, liver |
| | | sample collection 2 hours after treatment |
| 3. EPSI, | 4 | 6-weeks HSD, i.p. peptide administration |
| 1 mg/kg, 2 h. | | at a dose of 1 mg/kg, insulin solution |
| | | injection, liver sample collection 2 |
| | | hours after treatment |
| 4. EPSI, | 5 | 6-weeks HSD, i.p. peptide administration |
| 1 mg/kg, 12 h. | | at a dose of 1 mg/kg, insulin solution |
| | | injection, liver sample collection 2 |
| | | hours after treatment |
| 5. EPSI, | 4 | 6-weeks HSD, i.p. peptide administration |
| 10 mg/kg, 2 h. | | at a dose of 10 mg/kg, insulin solution |
| | | injection, liver sample collection 2 |
| | | hours after treatment |
| 6. EPSI, | 5 | 6-weeks HSD, i.p. peptide administration |
| 10 mg/kg, 12 h. | | at a dose of 10 mg/kg, insulin solution |
| | | injection, liver sample collection 2 |
| | | hours after treatment |

The following proteins were analyzed in liver samples by Western Blot analysis (WB): P-Akt Ser473, P-Akt Thr308, P-p70 S6 Kinase (Thr421/Ser424). Results were normalized to expression levels of reference protein GAPDH. The calculation was performed using the Image Lab 5.2.1 software (BioRad Inc., USA).

Antibodies used (manufactured by Cell Signaling Technology, Inc):
Phospho-Akt (Ser473) (D9E) XP® Rabbit mAb #4060
Phospho-Akt (Thr308) (D25E6) XP® Rabbit mAb #13038
Phospho-p70 S6 Kinase (Thr421/Ser424) Antibody #9204
GAPDH (14C10) Rabbit mAb #2118

### 12.2.2. Animals

12.2.2.1. Species, sex, age, number of animals

Stage 1. Sprague-Dawley male rats (N=50) were used in this study. The body weight at the beginning of the experiment was about 400 g. The animals were obtained after the RC VEC No. study under protocol 165 in agreement with the veterinarian.

Stage 2 and 3. Sprague-Dawley male rats (N=50) were used after stage 1). Bodyweight at the beginning of this stage was about 400-450 g. All animals were free of species-specific pathogens (SPF status according to the FELASA list, 2014).

### 12.2.2.2. Animal Source

Nursery of laboratory animals Charles River Laboratories Ink.

### 12.2.2.3. Adaptation

All tests and operations with animals were performed at the end of the adaptation period, which is more than 14 days after the previous manipulations, according to Protocol No. 165.

### 12.2.2.4. Housing

The animals were kept in the RC VEC; in conditions of free access to water and food, with a light mode 12/12 (light turns on at 09:00), in rooms with an air exchange rate of at least 15 rev/h, an air temperature of 20-24° C., humidity 30-70%. Animals were kept in groups of 5 cages in accordance with the seating standards produced by Techniplast (Italy), and before the start of stage 2, the animals were placed 2-3 per cage. The animals had unlimited access to food (granulated autoclavable feed produced by Tosno feed mill, Russia) and water free of microorganisms throughout the study. Lignocel BK 8/15 wood chips (Safe, France) were used as bedding. Requirements for drinking water for animals and the microbiological safety of water, feed, and bedding in the RC VEC. All materials supplied to the animals were decontaminated.

### 12.2.3. Test Description

### 12.2.3.1. Glucose Tolerance Test.

This technique is used to assess the ability of a test substance to affect the metabolism of carbohydrates, including glucose. For this purpose, the animals were deprived of food for 12 hours. Then a glucose solution was injected i.g. at a dose of 2 g/kg. Using a glucose meter (for example, ACCU-CHEK Advantage II Glucose Monitor, Roche, IN, United State; or a similar model), the glucose concentration in a blood drop obtained from the tail vein was measured at 0, 15, 30, 60, and 120 minutes after administration glucose. The data obtained were used to plot the dependence of blood glucose concentration on time, and the area under the curve (AUC) was also calculated (Ren et al. (2019) Novel GLP-1 Analog Supaglutide Stimulates Insulin Secretion in Mouse and Human Islet Beta-Cells and Improves Glucose Homeostasis in Diabetic Mice. Front Physiol. 25; 10:930.).

### 12.2.3.2. Development of Metabolic Disorder Caused by the HSD.

This technique is used to develop a metabolic disorder in animals by providing a source of carbohydrates (sucrose) to the standard diet. The animals were provided with a 30% sucrose solution in addition to water. From day eight, the weight of the animals was measured once every two days and the amount of liquid drunk. In addition, once a week (8, 15, 22, 28 days), the blood glucose concentration was measured in animals using an ACCU-CHEK Advantage II Glucose Monitor (Roche, IN, United States). A metabolic disorder was formed after a significant increase in blood glucose (for two consecutive measurements) relative to the values for the control group. It is important to note that this method was not a way to develop obesity or diabetes. Instead, the goal was to develop symptomatic mild disorders of carbohydrate metabolism (in particular glucose) and induce the development of glucose tolerance (Apolzan et al. (2012) Differential effects of chow and purified diet on the consumption of sucrose solution and lard and the development of obesity. Physiol Behav. 18; 105(2):325-31).

12.2.3.3. The Injection of Substances into the Vena Cava

Insulin solution (10 U/kg body weight, volume 100 μl/kg body weight, Kanisulin, 40 U/ml, Intervet International Gmb, Germany) was injected into the inferior vena cava, followed by liver tissue sampling. For this, animals were subjected to anesthesia with a combination of Zoletil and Xylazine. Then, using scissors, a longitudinal incision was made on the abdominal side, the contents of the abdominal cavity were gently pushed aside. The rat vena cava is a large vessel that runs along the spine. Next, the insulin solution was injected using 0.5 ml syringes with a 30G needle. (Wang et al. (1999) In vivo insulin signaling in the myocardium of streptozotocin-diabetic rats: opposite effects of diabetes on insulin stimulation of glycogen synthase and c-Fos. Endocrinology. 140(3):1141-50). At the end of the procedures, the animals were euthanized with an excess amount of injection anesthesia with a combination of Zoletil and Xylazine, i.e., manipulations were performed without the animal coming out of anesthesia.

### 12.2.3.4. The Analysis of Serum Insulin Concentrations

The insulin concentration in the blood serum samples of animals was measured using the enzyme-linked immunosorbent assay using a standard kit (Rat Insulin ELISA (10-1250-01), Mercodia, USA), according to manufacturer protocol.

### 12.2.3.5. The Analysis of Blood Lipid Profile

Glucose, triglycerides, cholesterol, LDL, HDL were measured using a laboratory biochemical analyzer BioSystems A25 (Spain) using standard analytical kits, according to manufacturer protocol.

### 12.2.3.6. Homeostasis Model Assessment of Insulin Resistance (HOMA-IR)

HOMA-IR was evaluated for potentially impaired glucose tolerance and diabetes. HOMA-IR is calculated as (Fasted glucose (mmol/L)*Fasted insulin (mU/L))/22.5.

### 12.2.4. Statistical Analysis

Statistical analysis was performed using nonparametric tests (Mann-Whitney) for samples with a distribution other than normal or using one-way analysis of variance (ANOVA) with post hoc comparison by Fisher's test for samples with a normal distribution. The results are presented as a mean±standard error of the mean (SEM). A difference between groups was considered significant at p<0.05.

### 12.3. Study Results

### 12.3.1. Stage 1

At this stage, the effects of EPSI (1 and 10 mg/kg) were evaluated in rats given the HSD for five weeks.

Animals under HSD develop a metabolic condition with increased glucose levels in the blood and insulin resistance.

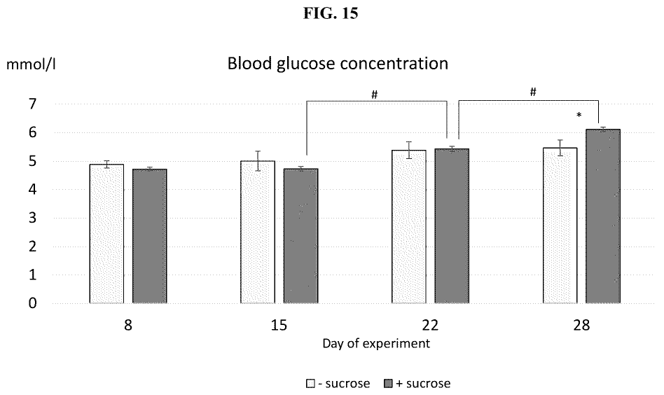

As shown in FIG. 15, a significant increment of blood glucose was noted at day 22 of HSD compared to day 15. Furthermore, a significant increase of blood glucose in the HSD group compared to the control group was obtained on day 28 of the diet. At the same time, no changes in animals' weight were found during the observation period.

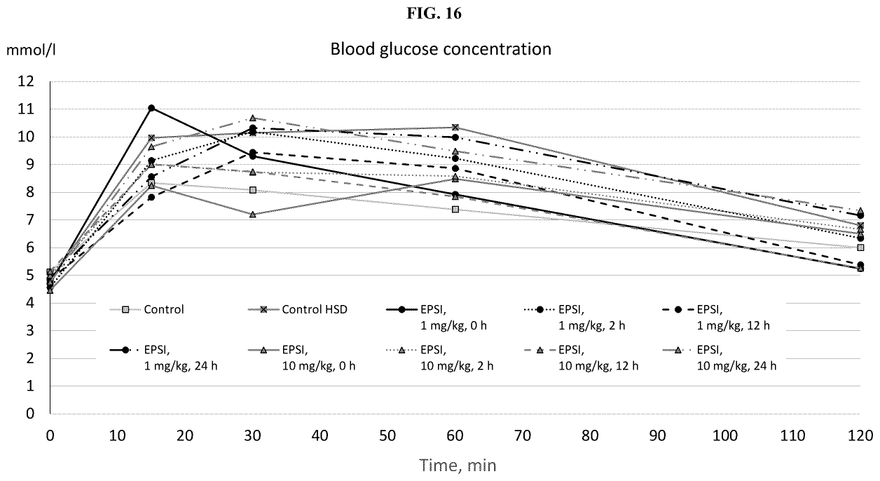

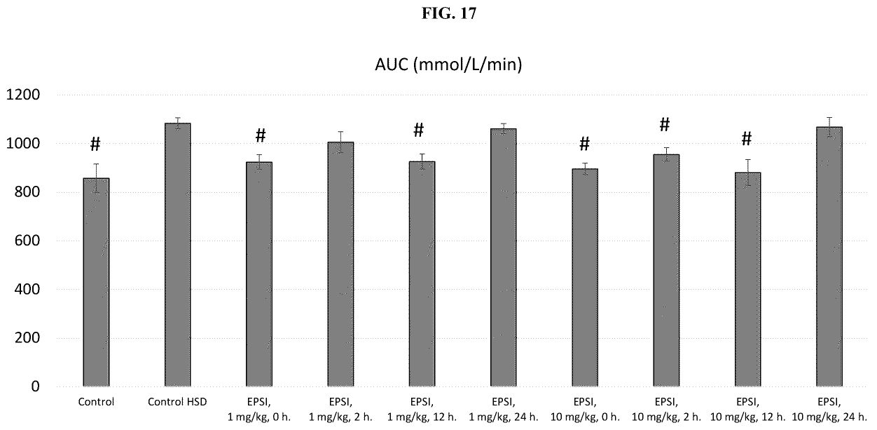

The glucose tolerance test showed a significant increase of AUC in HSD animals receiving saline compared to control rats (1083.2±22.89 mmol/L/min and 857.4±58.76 mmol/L/min, respectively) (FIG. 16). This indicates a glucose tolerance in animals given the HSD. Single EPSI administration was potent at reducing overall glucose concentration in the blood according to AUC values (FIG. 17). Compared to the HSD group, significant differences were shown for 1 and 10 mg/kg EPSI administered 12, 24 hours prior to and right before glucose injection.

Thus, the results indicate the hypoglycemic effect of EPSI peptide after single administration in the model of HSD-induced metabolic condition.

### 12.3.2. Stage 2

At this stage, the effects of EPSI (1 and 10 mg/kg) on blood serum insulin levels and lipid profile were evaluated in rats given the HSD for six weeks.

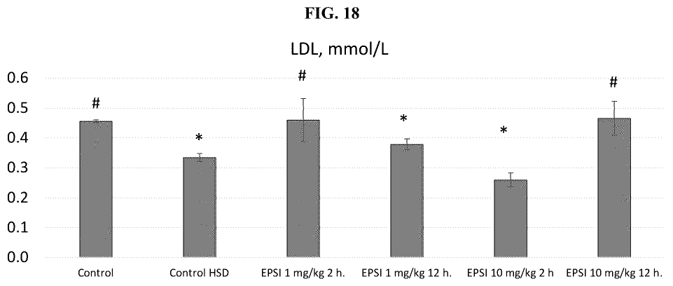

Chronic HSD led to a decrease in LDL (FIG. 18) in blood serum of rats. An increase in LDL relative to the “HSD Control” group (0.33±0.013 mmol/L) was observed for the 1 mg/kg EPSI, 2 h (0.45±0.072 mmol/L) and for 10 mg/kg EPSI, 12 h (0.46±0.057 mmol/L).

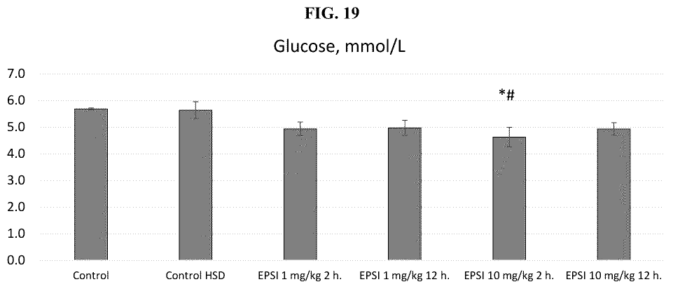

Blood serum glucose level was lower only in the 10 mg/kg EPSI, 2h group compared to the HSD Control group (FIG. 19).

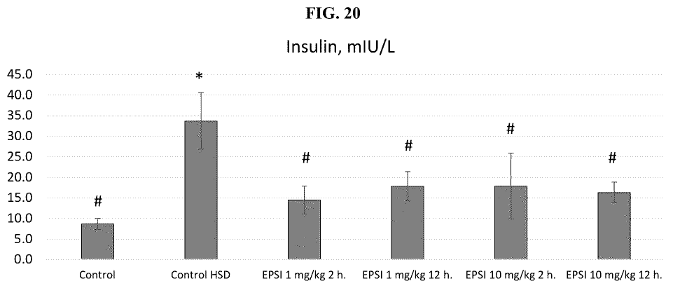

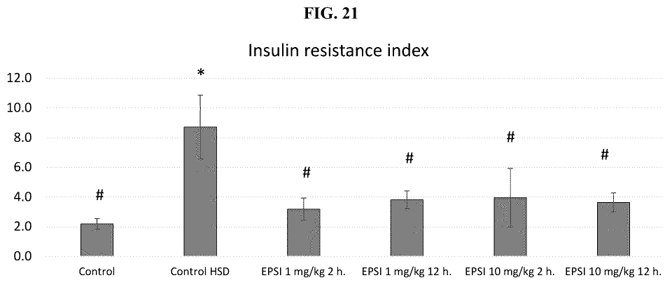

Insulin concentration and insulin resistance index HOMA-IR in the HSD Control group were significantly higher than similar values in the Control group, which confirms the presence of symptoms of insulin resistance in experimental animals caused by maintenance caused by the HSD (FIG. 20). EPSI administration in a dose of 1 and 10 mg/kg when administered 2 and 12 h before blood sampling led to a decrease in insulin concentration and the HOMA-IR (FIG. 21).

### 12.3.3. Stage 3

At this stage, the effects of EPSI (1 and 10 mg/kg) on the phosphorylation levels of proteins participating in intracellular insulin signaling in the liver of animals with symptoms of metabolic disorder were studied.

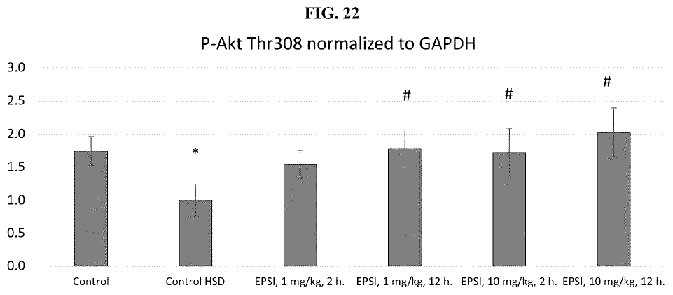

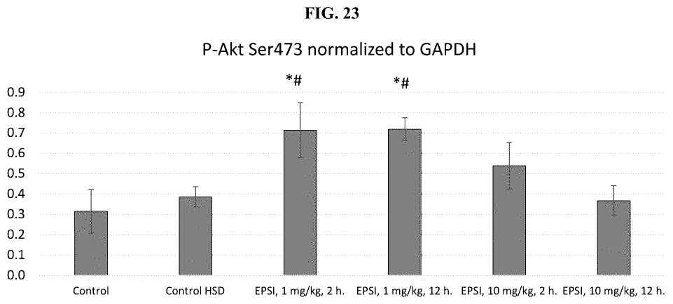

HSD led to a decrease in the level of AKT phosphorylation by Thr308 in liver cells in response to insulin administration compared to the control group on standard diet (FIG. 22). This result indicates impaired insulin signaling and the development of insulin resistance. The lack of the HSD effect on the phosphorylation level of p-AKT by Ser473 (FIG. 23) may be, without wishing to be bound by theory, due to the low severity of the pathological condition obtained in the HSD model.

The administration of the EPSI peptide to animals on the HSD resulted in:
Restoration of the level of p-AKT (Thr308) to the control level, which potentially indicates the ability of the peptide to normalize disturbances in insulin signaling and alleviate insulin resistance.
An increase in the level of p-AKT (Ser473) indicates an increase in the efficiency of insulin signaling and indicating a therapeutic effect in a more severe pathological condition.

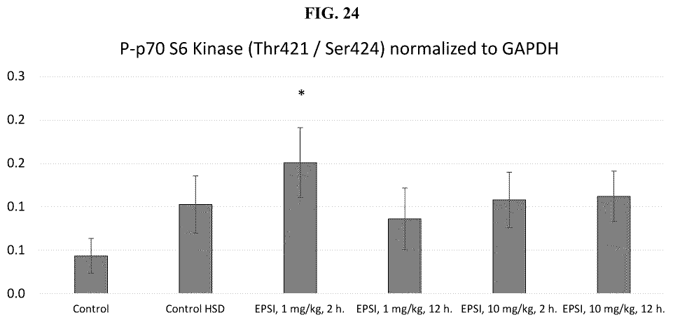

The level of the phosphorylated form p70S6k1 (Thr421/Ser424) was increased on a trend level in all groups of HSD animals compared to Control values (FIG. 24), showing the development of insulin resistance. EPSI administration does not lead to an even greater increase in p70S6k1 (Thr421/Ser424), despite the induction of the levels of p-AKT Ser473 and Thr308 (which could lead to the activation of p70S6k1 through mTORC1). This result is likely to indicate the absence of compensatory hyperinhibition of the insulin cascade (“rollback effect”) at a longer time after peptide administration.

### 12.4. Discussion

Rats kept on the HSD for more than five weeks developed signs of metabolic pathology. They showed an increased insulin concentration and HOMA-IR after overnight fasting. The AUC of blood glucose concentration was also significantly higher in the HSD group in the glucose tolerance test. A single i.p. administration of the EPSI peptide at a dose of 1 and 10 mg/kg at different time points (0 h, 2 h, 12 h, 24 h before glucose administration) was accompanied by a normalization of the glucose concentration AUC, insulin concentration and HOMA-IR to the control values.

The analysis of rat liver tissue samples using WB showed the ability of the EPSI peptide to induce the levels of p-AKT Ser473, and Thr308 in rats kept on the HSD. These changes follow the observed decrease in the HOMA-IR and can partially explain the mechanism of this effect.

In general, the results obtained indicate that EPSI activates intracellular processes in the liver, which are responsible for alleviating the state of insulin resistance, and the mechanism correlates well with data previously obtained in vitro:

Activation of p-AKT should be expressed in inhibition of GSK3p, which correlates with a decrease in the level of GSK3p expression under the influence of the EPSI peptide, which we observed in an experiment on mouse fibroblasts (see Example 3).

Activation of p-AKT in the absence of p70S6k1 activation indicates the induction of the IRS/PI3K/AKT cascade, which could supposedly be consistent with the data on the EPSI-induced increase in the level of IRS2 expression in mouse fibroblasts (see Example 5).

The effect of EPSI on insulin signaling components in the liver has a pronounced therapeutic focus. It is an additional argument favoring EPSI for alleviating pathologies associated with insulin resistance.

### 12.5. Illustrative Conclusions

1) A single i.p. injection of the EPSI peptide at a dose of 1 and 10 mg/kg was accompanied by a pronounced hypoglycemic effect in the glucose tolerance test in rats under HSD.

2) A single i.p. administration of the EPSI peptide at a dose of 1 and 10 mg/kg 2 h and 12 h before blood collection, normalized insulin concentration, and HOMA-IR altered by HSD.

3) EPSI administration led to the normalization of the pAKT (Thr308) level and induction of the p-AKT (Ser473) level without affecting p70S6k1 (Thr421/Ser424).

4) EPSI acts as an activator of the insulin receptor cascade.

### Example 13. The Study of the Effects of EPSI Peptide on the Severity of Symptoms of Metabolic Disorder in Laboratory Mice Caused by a High-Fat Diet

### 13.1. Study Objective

The study objective was to evaluate the effect of EPSI peptide administration on the severity of symptoms of metabolic disorder in laboratory C57BL/6 mice on a high-fat diet (HFD) and compare the results with the effects of positive control drug Metformin.

### 13.2. Materials and Methods

### 13.2.1. Animals and Housing

One hundred sixty-five male C57BL/6 mice aged 6-7 weeks were obtained from IBCh RAS nursery of laboratory animals “Pushchino”. The mean body weight before the experiment was 20.2±0.3 grams.

The animals were kept in a barrier-type animal-keeping room (barrier zone 2 LBT IBCh RAS, room number: 1.7). The housing and all procedures were carried out following the standards defined by the Directive 2010/63/EU on the protection of animals used for scientific purposes and the Guide for Care and Use of Laboratory Animals (National Academy Press, Washington D.C., 2011).

The animals were housed under controlled environmental conditions (temperature 20-24° C., relative humidity 30-70%, 12-hour lighting cycle (08:00-20:00—“day”, 20:00-08:00—“night”) and at least 10-fold change in the room air volume per hour). Temperature and humidity were constantly monitored in each animal-keeping room automatically using the Eksis Visual Lab system (EVL, Praktik-NTs OJSC).

### 3.2.2. Feed

A standard laboratory rodent pellet SNIFF RI/M-H V1534-30 (58% carbohydrates, 9% fat, 33% protein, 306 kcal/100 g) was given ad libitum to the control animals. For HFD groups, the high-fat feed was used. For 1 kg of feed, 610 g of ground SNIFF feed and 360 g of melted pork lard were taken, water (˜250 ml, 60-70° C.), 10 g of sodium chloride, 30 g of sodium glutamate were added. The mixture was brewed to a dough consistency, and food granules were formed. Then the granules were dried at 60-70° C. for 10-12 hours. The finished food for consumption was transferred to the animal-keeping area. The prepared food was stored at 4° C. for no more than 7 days. The approximate energy value of the HFD feed was 516 kcal/100 g (45% fat, 35% carbohydrates, 20% protein).

### 13.2.3. HFD Model of Metabolic Syndrome (MS)

The metabolic syndrome was modeled by keeping animals on a HFD for 16 weeks. The control group was kept on a standard diet (STD) during this period. Animals had free access to feed and water located in the cage lid.

### 13.2.4. Treatment Groups

The study was carried out in 3 Stages.

At stage 1 of the study, to replicate the metabolic syndrome, 25 mice were kept on a STD (group 1), 140 mice—on a HFD (groups 2-5). The animals' body weight was monitored weekly. By the end of the 16th week, mice with the maximum gain were selected among the animals kept on a HFD, and four groups of 25 animals were formed.

At stage 2, the effects of acute EPSI administration (groups 3 and 4) and metformin (group 5) were studied in the glucose tolerance test. On the day of the experiment, animals from groups 3-5 were injected with the tested substances: one half of the group (12 or 13 animals) 2 hours before the experiment and the other half—12 hours. Group 1 and 2 (STD control and HFD control) were injected with saline (Table 5).

At stage 3, the effects of chronic administration for 28 days of EPSI and metformin were studied. Groups of 10 animals were formed from the existing groups from Stage 2, with similar mean body weight (Table 6). The animals' bodyweight was monitored three times a week, water, and feed consumption—weekly. After the last treatment, the animals were euthanized for necropsy and their blood collected. The internal organs were examined for the presence of macro damage and weighed. The adipose tissue surrounding the epididymis was evaluated as a marker of obesity in animals. For histological examination, samples of adipose tissue and liver tissue were fixed in 10% formalin solution for further histological analysis.

TABLE 5
Stage 2 experimental groups

| | | | | | Drug | |
|---|---|---|---|---|---|---|
| | | | | | adminis- | |
| | | | | | tration | |
| | Diet/ | | Drug | | before | N of |
| No | Group | Treatment | dose | | the test | animals |
| 1 | STD | Single i.p. | | | 2/12 h | 12/13 |
| | Control | saline | | | | (total 25) |
| 2 | HFD | Single i.p. | | | | |
| | Control | saline | | | | |
| 3 | HFD | Single i.p. | 5 | mg/kg | | |
| | EPSI | EPSI | | | | |
| 4 | HFD | Single i.n. | 5 | mg/kg | | |
| | EPSI | EPSI, i.p. | | | | |
| | | saline | | | | |
| 5 | HFD | Single peroral | 250 | mg/kg | | |
| | Metformin | (p.o.) | | | | |
| | | Metformin + | | | | |
| | | i.p. saline | | | | |

TABLE 6
Stage 3 experimental groups

| No | Diet/Group | N of animals | Treatment | Drug dose | |
|---|---|---|---|---|---|
| 1 | STD | 10 | Chronic i.p. saline | | |
| | Control | | for 28 days | | |
| 2 | HFD | 10 | Chronic i.p. saline | | |
| | Control | | 28 days | | |
| 3 | HFD | 10 | Chronic i.p. EPSI | 5 | mg/kg |
| | EPSI | | 28 days | | |
| 4 | HFD | 10 | Chronic i.n. EPSI + | 5 | mg/kg |
| | EPSI | | i.p. saline for 28 | | |
| | | | days | | |
| 5 | HFD | 10 | Chronic peroral | 250 | mg/kg |
| | Metformin | | (p.o.) Metformin + | | |
| | | | i.p. saline for 28 | | |
| | | | days | | |

The administration volume was 10 ml/kg for drugs administered i.p., 6 μl/animal for i.n. administration and 5 ml/kg for drugs administered p.o.

### 13.2.5. Procedures

### 13.2.5.1. Food Deprivation

The animals were deprived of food for 12 hours before the glucose tolerance test and euthanasia. Animals had free access to water.

### 13.2.5.2. Bodyweight

The animals were weighed weekly on weeks 1-16 of the experiment (1-120 days of the study), during the administration of the tested substances (121-148 days)—three times a week. The bodyweight of a hungry animal immediately before necropsy was taken to calculate the percentage of organ weight to body weight.

### 13.2.5.3. Feed and Water Consumption

Each animal's food and water consumption was evaluated weekly by weighing the grid with food or a bottle of water before and after 24 hours.

### 13.2.5.4. Glucose Tolerance Test

The animals were deprived of food for 12 hours before the test. Baseline glucose level was measured at the end of fasting (0 min time point) in the blood drop taken from the tip of the tail by incision (with a needle) of the tail vein. The test substances were administered according to the treatment group. Half of the animals received drug injections 2 hours, and the other half—12 hours before the test. The glucose concentration in the blood was determined 15, 30, 60, 120 minutes after i.p. 40% glucose solution administration in a dose of 2 mg/kg with Satellite Express (Elta, Russia) test system.

### 13.2.5.5. Euthanasia and Blood Samples Collection

At the end of the study, the animals were euthanized. The animal was anesthetized with an injection of Zoletil/Xylazine, after which a terminal blood sample was taken from the inferior vena cava. A blood sample (about 0.8 ml) was collected in a test tube. After blood clotting, the samples were centrifuged to obtain serum, aliquoted in the required volumes, frozen, and stored at −20° C. until analysis.

### 13.2.5.6. Evaluation of Blood Glucose Concentration

Blood serum was analyzed for glucose concentration using the Randox GB reagent kit on an automatic biochemical analyzer Sapphire-400 (Tokyo Boeki LTD, Japan), following the manufacturer's instructions.

### 13.2.5.7. Evaluation of Insulin Concentration in Blood Serum

Insulin concentration in blood serum samples was measured by enzyme immunoassay using a standard commercial kit, following the manufacturer's instructions. Analysis was performed using a Multiskan™ GO spectrophotometer (Thermo Scientific). Based on the obtained data on the concentration of insulin and glucose measured in animals before necropsy, the derived parameter of insulin resistance HOMA-IR was calculated using the formula:

HOMA-IR=(glucose [mmol/L]×insulin [μU/ml])/22.5.

### 13.2.5.8. Evaluation of Pro-Inflammatory Cytokines in Blood Serum

The concentration of proinflammatory cytokine TNFa and IL1 was measured in blood serum samples by ELISA using standard commercial kits, following the manufacturer's instructions. Analysis was performed using a Multiskan™ GO spectrophotometer (Thermo Scientific).

### 13.2.6. Pathomorphological Evaluation and Histology

### 13.2.6.1. Organ Mass

After necropsy, the following internal organs were weighed: adrenal glands, kidneys, liver, pancreas, heart, spleen, and testicles.

### 13.2.6.2. Necropsy and Tissue Collection

During necropsy, the body's external state, internal surfaces, the cranial cavity, chest, abdominal and pelvic cavities with organs and tissues located in them, the neck with organs and tissues, the skeleton, and the musculoskeletal system were examined. The organs and tissues were fixed in 10% formalin. Liver and adipose tissue samples were weighed and stored in 10% formalin for subsequent histological analysis.

### 13.2.6.3. Histology

Histological analysis of liver and adipose tissue was performed for all euthanized animals. For this, the fixed tissues were dehydrated, soaked in paraffin. The paraffin blocks were then cut into sections. Sections were standardly stained with hematoxylin and eosin. Tissue specimens were examined by light microscopy.

Microscopic analysis was performed using a DMLA Leica transmitted light microscope (Germany), a Photometrics Cool SNAP cf video camera (USA), and Mekos software (Russia). Estimated parameters were the size of lipid granules in the cytoplasm of hepatocytes (in microns), the linear size of visceral fat adipocytes (in microns), thickness of the subcutaneous layer of parietal fat (in microns). One hundred adipocytes were measured on sections of the visceral fat samples in 15-20 random fields of view.

### 13.2.7. Statistical Analysis

The quantitative data were analyzed by one-way analysis of variance ANOVA, repeated-measures ANOVA, or nonparametric Kruskal-Wallis analysis. When the statistical analysis showed a significance level of P less or equal to 0.05, the Fischer test or Mann-Whitney test was used to compare groups. Statistical analysis was performed using the Statistica 7.1 software. Differences were determined at the 5% significance level.

### 13.3. Results

### 13.3.1. Inspection of Animals

HFD for 16 weeks resulted in a significant increase in body weight in 75% of animals. Five mice from the HFD group died during this period. The most common complication in experimental metabolic syndrome is ruptured kidneys, which could have caused the animals' death. Also, in group 5, after a single injection of Metformin, one animal died on the 116th day of the study (on the 3rd day after the glucose tolerance test), one died on the 127th day of the study (on the 5th day of chronic administration of Metformin). The cause of death is associated with the effect of the tested drug since deviations in the state of health of the animals were observed immediately after the first injection (hunching, tremor, weight loss). In group 2 (HFD control), one animal died on the 133rd day of the study. Dead animals were replaced with animals left over after forming groups and entered into the experiment according to the study schedule.

Animals' bodyweight at the beginning of drug treatment is shown in Table 7.

TABLE 7
Animal body weight before the administration of the tested substances, grams

| | Group 1 | Group 2 | Group 3 | Group 4 | Group 5 |
|---|---|---|---|---|---|
| Day | STD Control | HFD Control | EPSI i/p | EPSI i/n | Metformin p.o. |
| 112 | 28.25 ± 0.361# | 36.67 ± 0.807* | 38.26 ± 0.933* | 37.89 ± 0.8* | 36.94 ± 0.954* |

*P < 0.05 denotes a significant difference from group 1;
#P < 0.05 - from group 2 (one-way ANOVA, Fisher test).
N = 25 in each group. The results are presented as the mean ± standard error of meant (SEM).

### 13.3.2. Glucose Tolerance Test (GTT)

Table 8 shows the glucose tolerance test results on the 113^{th} day of the study.

TABLE 8
The glucose concentration (mmol/l) 0, 15, 30, 60, and 120 min after
2 mg/kg glucose injection in GTT 2 and 12 hours after drug treatment.
2 h post-treatment

| Group / time | | | | | | AUC (mmol/ |
|---|---|---|---|---|---|---|
| (min) | 0 (basal) | 15 | 30 | 60 | 120 | l/ min) |
| 1. STD | 8.1 ± 0.3# | 18.8 ± 0.8# | 16.4 ± 0.9# | 11.2 ± 0.6# | 8.5 ± 0.3# | 1475 ± 50# |
| Control | | | | | | |
| 2. HFD | 10.3 ± 0.3* | 24.5 ± 0.8* | 23.8 ± 1.9* | 17.1 ± 1.6 | 11.1 ± 0.5* | 2093 ± 124* |
| Control | | | | | | |
| 3. EPSI i.p. | 9.8 ± 0.4* | 20.6 ± 1.0# | 21.4 ± *1.2 | 17.0 ± 1.1* | 11.6 ± 1.1* | 1984 ± 97* |
| 4. EPSI i.n. | 10.6 ± 0.4* | 19.7 ± 0.8# | 21.3 ± 1.3* | 16.4 ± 0.9* | 10.6 ± 0.3 | 1919 ± 54* |
| 5. Metformin | 7.2 ± 0.374# | 17.61 ± 1.555# | 21.68 ± 2.106* | 15.55 ± 1.46* | 10.08 ± 0.875 | 1808 ± 131* |

12 h post-treatment

| Group / time | | | | | | AUC (mmol/ |
|---|---|---|---|---|---|---|
| (min) | 0 | 15 | 30 | 60 | 120 | l/ min) |
| 1. STD | 8.0 ± 0.1# | 15.3 ± 0.9# | 11.6 ± 0.4# | 9.1 ± 0.40# | 8.9 ± 0.5 | 1231 ± 35# |
| Control | | | | | | |
| 2. HFD | 9.6 ± 0.4* | 23.4 ± 0.9* | 21.5 ± 1.4* | 16.6 ± 1.32* | 10.4 ± 0.3 | 1971 ± 92* |
| Control | | | | | | |
| 3. EPSI i.p. | 9.3 ± 0.2 | 23.0 ± 0.8* | 23.2 ± 1.1* | 18.6 ± 1.18* | 11.8 ± 0.76* | 2132 ± 85* |
| 4. EPSI i.n. | 9.2 ± 0.3 | 21.7 ± 1.0* | 20.1 ± 1.4* | 14.5 ± 1.29* | 10.0 ± 0.6 | 1803 ± 97* |
| 5. Metformin | 7.0 ± 0.7# | 19.6 ± 1.3*# | 20.5 ± 1.8* | 15.7 ± 1.8* | 11.2 ± 1.4 | 1857 ± 149* |
| p.o. | | | | | | |

*P < 0.05 denotes a significant difference from group 1;
#P < 0.05 - from group 2 (one-way ANOVA, Fisher test).
N = 25 in each group.
The results are presented as the mean ± SEM.

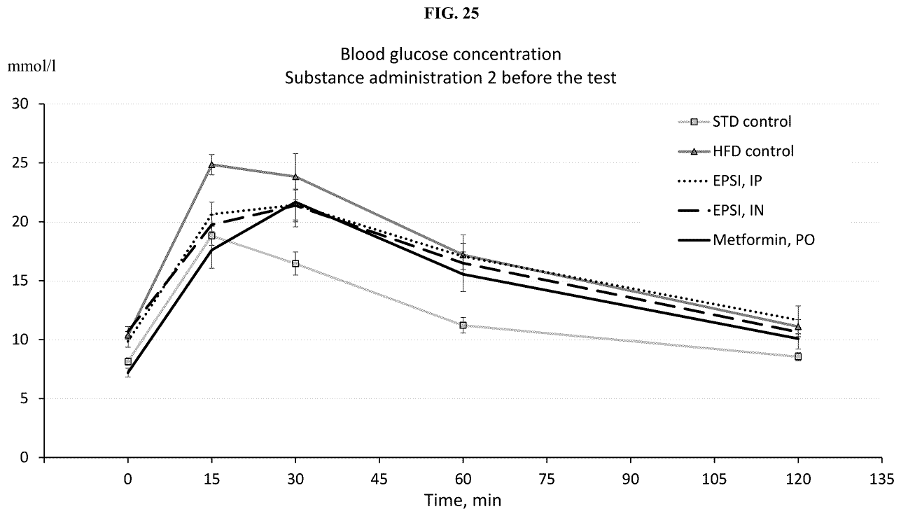

The mice kept on HFD had increased basal glucose levels. The exception was group 5, which was injected with metformin—the glucose concentration at 0 min of the test did not differ from the level in the STD control group. After glucose injection, all mice showed hyperglycemia at 15 minutes; the highest values were observed in the HFD group, injected with the solvent (group 2). Significantly increased glucose concentrations persisted up to 60 minutes in groups 2-5. By 120 minutes of the test, there was a recovery to the initial values in all groups (FIG. 25).

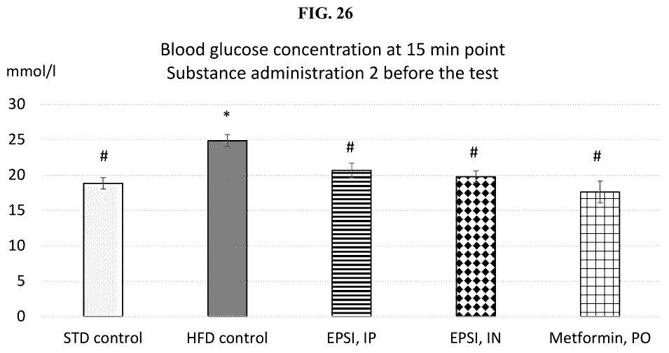

The drugs' effects were most clearly manifested when administered 2 hours before the test (FIG. 26). The single p.o. administration of the comparison drug “Metformin” at a dose of 250 mg/kg and i.p. and i.n. administration of the EPSI peptide at a dose of 5 mg/kg reduced the peak value of blood glucose concentration at 15 minutes after the glucose administration.

### 13.3.3. Bodyweight

The mean body weight of mice is presented in Table 9. The chronic administration of the test substances started on day 122 of the study.

TABLE 9
Bodyweight during the period the test substance administration, grams

| | 1 | 2 | 3 | 4 | 5 |
|---|---|---|---|---|---|
| Day | STD Control | HFD Control | EPSI i.p. | EPSI i.n. | Metformin p.o. |
| 122 | 28.4 ± 0.3# | 34.6 ± 1.3* | 36.3 ± 0.8* | 36.2 ± 0.8* | 34.3 ± 0.7* |
| 124 | 28.8 ± 0.4# | 35.4 ± 0.7* | 36.7 ± 0.8* | 36.0 ± 0.9* | 33.9 ± 0.7* |
| 126 | 28.6 ± 0.4# | 35.1 ± 0.7* | 35.9 ± 0.7* | 35.4 ± 0.9* | 33.4 ± 0.8* |
| 129 | 28.8 ± 0.4# | 34.6 ± 0.7* | 35.7 ± 0.6* | 35.3 ± 0.9* | 32.9 ± 0.7* |
| 131 | 28.8 ± 0.4# | 34.9 ± 0.9* | 35.8 ± 0.7* | 35.2 ± 1.0* | 33.1 ± 0.7* |
| 133 | 28.7 ± 0.3# | 34.9 ± 1.1* | 35.8 ± 0.8 | 35.3 ± 1.3* | 33.1 ± 0.8* |
| 136 | 29.5 ± 0.8# | 35.2 ± 0.9* | 35.9 ± 0.8 | 35.0 ± 1.2* | 32.4 ± 1.1* |
| 138 | 28.3 ± 0.4# | 35.1 ± 0.9* | 35.9 ± 0.8* | 34.5 ± 1.1* | 32.2 ± 1.1* |
| 141 | 28.5 ± 0.3# | 35.1 ± 1.0* | 36.0 ± 0.8* | 34.3 ± 1.2* | 32.9 ± 0.9* |
| 144 | 28.5 ± 0.3# | 35.5 ± 1.1* | 36.4 ± 0.8* | 34.6 ± 1.2* | 33.2 ± 1.0* |
| 147 | 28.4 ± 0.3# | 35.6 ± 1.2* | 36.6 ± 0.9* | 34.9 ± 1.4* | 33.5 ± 1.0* |

*P < 0.05 denotes a significant difference from group 1;
#P < 0.05 - from group 2 (repeated measures ANOVA, Fisher test).
N = 10 in each group. The results are presented as the mean ± SEM.

At the beginning of the drug treatment (day 122), animals' bodyweight kept on the HFD was significantly higher by more than 29% than in the STD control group. Animals from the experimental groups (3, 4, 5) did not differ from the HFD Control group initially.

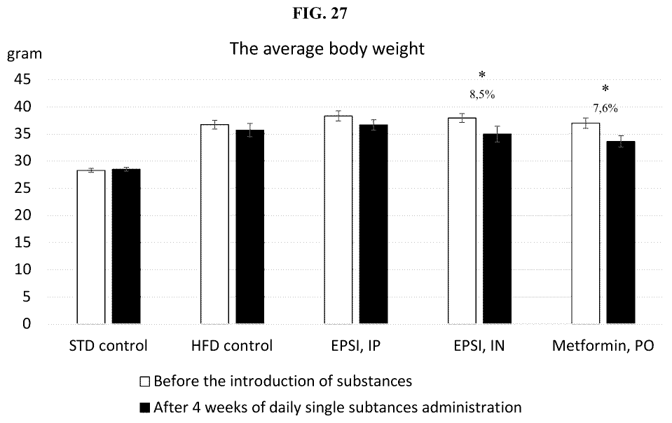

FIG. 27 shows the average body weight values before and after the drug administration. Chronic 4-week p.o. administration of the comparison drug “Metformin” led to a significant decrease in the weight by 7.6% compared to the values obtained before the substance administration. I.n. administration of the EPSI peptide led to a decrease in mice's weight by 8.5% (FIG. 27).

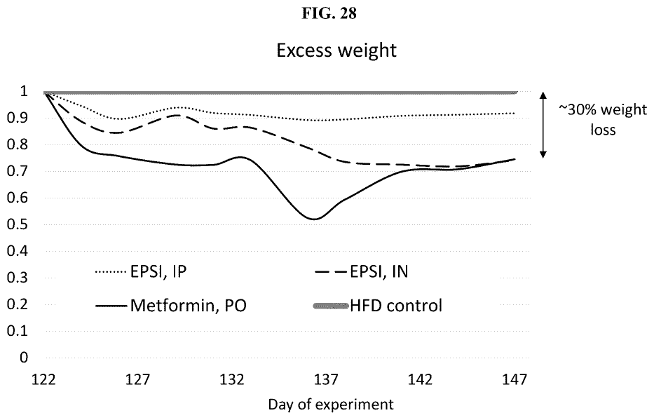

Excess weight was calculated as the difference between the mean values in the treatment and STD control groups on each day normalized to HFD control values (FIG. 28). Chronic i.p. administration of the peptide resulted in a 10% loss of excess body weight. I.n. administration of EPSI peptide resulted in a 30% loss of excess body weight, and this result was comparable to the effect of metformin chronic administration.

### 13.3.4. Water and Feed Consumption

Group average water and food intake are presented in Tables 10-11, respectively.

TABLE 10
Weekly water consumption, ml/kg/day.

| | 1 | 2 | 3 | 4 | 5 |
|---|---|---|---|---|---|
| Week | STD Control | HFD Control | EPSI i.p. | EPSI i.n. | Metformin p.o. |
| Week 1 | 172.9 ± 13.5 | 144.2 ± 15.4 | 123.5 ± 13.2* | 139.8 ± 12.4 | 157.7 ± 15.7 |
| Week 2 | 223.9 ± 12.7# | 160.3 ± 15.1* | 130.8 ± 13.7* | 147.5 ± 14.3* | 172.7 ± 13.6* |
| Week 3 | 241.9 ± 15.6# | 180.8 ± 19.9* | 146.9 ± 11.5* | 173.8 ± 15.5* | 216.7 ± 24.3 |
| Week 4 | 291.3 ± 21.1# | 207.1 ± 26.7* | 152.6 ± 11.7* | 205.5 ± 20.5* | 262 ± 26.3 |

*P < 0.05 denotes a significant difference from group 1;
#P < 0.05 - from group 2 (repeated measures ANOVA, Fisher test).
N = 10 in each group. The results are presented as the mean ± SEM.

TABLE 11
Weekly food consumption, kcal/kg/day.

| | 1 | 2 | 3 | 4 | 5 |
|---|---|---|---|---|---|
| Week | STD Control | HFD Control | EPSI I/P | EPSI I/N | Metformin p.o. |
| Week 1 | 506.5 ± 12.1 | 436.1 ± 29.1 | 356.3 ± 13.7* | 362.5 ± 22.5* | 458.4 ± 55.4 |
| Week 2 | 558.0 ± 26.0 | 648.1 ± 67.9 | 635.4 ± 32.1 | 621.9 ± 50.4 | 758.4 ± 51.4* |
| Week 3 | 521.3 ± 20.8 | 511.9 ± 37.2 | 526.3 ± 16.8 | 512.1 ± 30.4 | 567.6 ± 21.3 |
| Week 4 | 563.3 ± 17.9# | 669.8 ± 34.6* | 600.6 ± 12.1# | 641.0 ± 15.1* | 709.4 ± 22.7* |

*P < 0.05 denotes a significant difference from group 1;
#P < 0.05 - from group 2 (repeated measures ANOVA, Fisher test).
N = 10 in each group. The results are presented as the mean ± SEM.

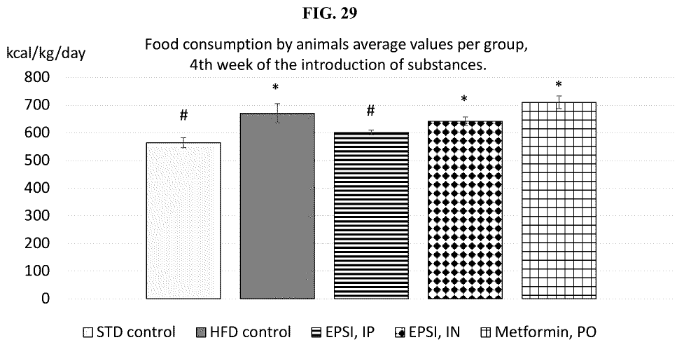

After two weeks of drug treatment, all groups on the HFD consumed less water than the STD control group. At the same time, there were no statistically significant differences in groups 3, 4, 5 in comparison to the HFD control group. In addition, food consumption in all experimental groups kept on the HFD did not significantly differ from the HFD control group in terms of the mean values for the first three weeks. However, the average food consumption on week four of observation showed that chronic i.p. administration of EPSI led to a decrease in feed consumption by more than 11% compared to the HFD control group (FIG. 29).

### 13.3.5. Glucose and Insulin Concentrations, HOMA-IR

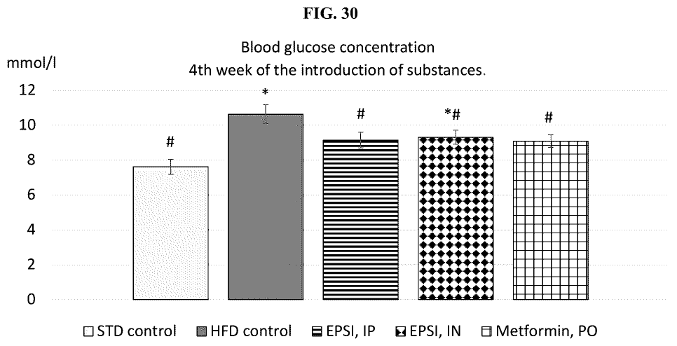

Four months of HFD led to an increase in glucose concentration in the blood. Chronic administration of metformin and EPSI administered i.p. and i.n. resulted in a decrease of this parameter compared to the HFD control group (FIG. 30).

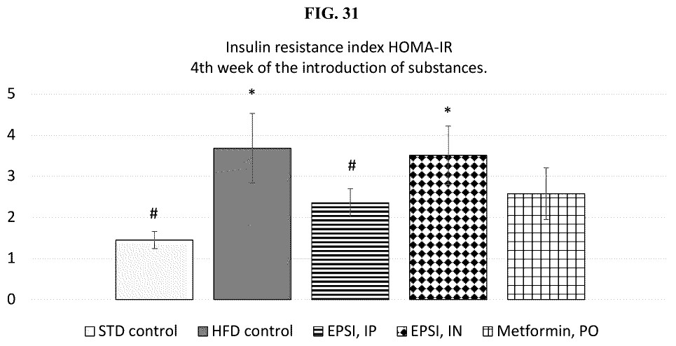

A detrimental effect of HFD on insulin and insulin resistance index was found, indicating a change in tissue sensitivity to insulin. Compared to STD control animals, HFD controls had a significant increase in HOMA-IR (FIG. 31). Four-week i.p. EPSI treatment resulted in the normalization of this parameter (FIG. 31).

### 13.3.6. Pro-Inflammatory Cytokines Concentration

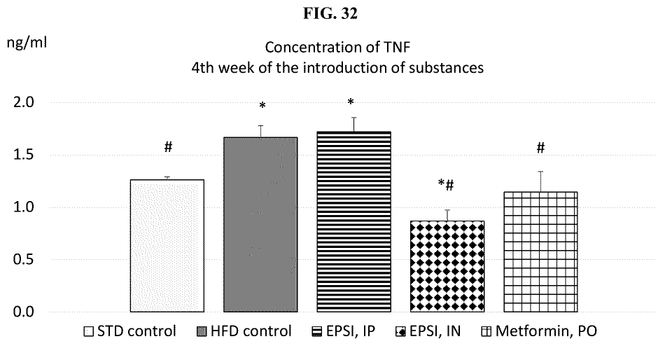

There was no change in IL-1 concentrations in blood serum observed in the HFD compared to the STD control group. At the same time, the concentration of the pro-inflammatory cytokine TNF-α in the blood serum of HFD control mice was significantly increased, indicating systemic inflammation in animals (FIG. 32). The four-week metformin reduced the concentration of TNF-α in comparison to HFD control group. Chronic i.n. administration of the EPSI peptide also decreased TNF-α concentration in the blood serum to the STD control level (FIG. 32).

### 13.3.7. Necropsy Results

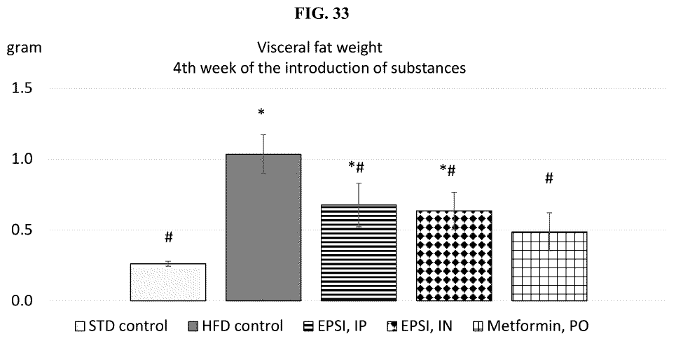

A necropsy study revealed an increased visceral fat (adipose tissue surrounding the epididymis) in the HFD control group (FIG. 33). Oral administration of metformin for four weeks led to a statistically significant decrease in visceral fat, and the same effect was found for both i.p. and i.n. EPSI administration (FIG. 33).

### 13.3.8. Histological Assessment

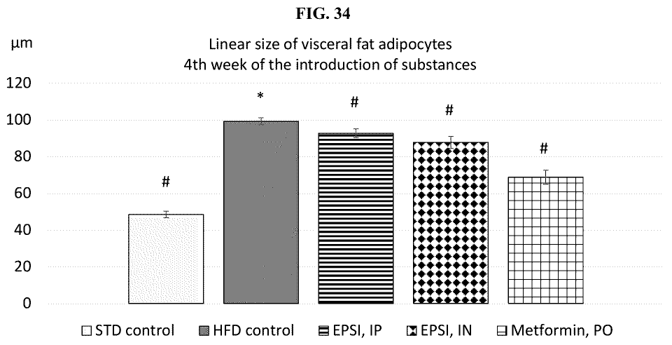

The formation of adipocytes occurs in the intrauterine period, starting from the last trimester of pregnancy and ending in the prepubertal period. After that, fat cells may increase in size with obesity or decrease with weight loss, but their number does not change throughout life. The thickness of parietal fat was measured on histological samples of subcutaneous adipose tissue. The morphometry data of the visceral fat is presented in FIG. 34.

Thus, under the HFD, the average size of fat cells exceeded those under the STD by an average of 1.75-2 times at different observation periods. Metformin administration for four weeks led to a statistically significant decrease in the average linear size of visceral fat adipocytes by 31% compared to HFD control. EPSI peptide treatment significantly reduced the size of adipocytes by more than 12% and 7% when administered i.n. and i.p., respectively (FIG. 34).

### 13.4. Discussion

This study aimed to investigate the efficacy of the EPSI peptide in a model of metabolic syndrome induced by HFD (45% fat content by weight). The effects of the peptide were studied after intraperitoneal and intranasal administration at a dose of 5 mg/kg. Metformin is a tableted antihyperglycemic drug for type 2 diabetes mellitus, and it was used as a comparison drug.

HFD increased the bodyweight of mice and led to the development of symptoms of hyperglycemia, hyperinsulinemia, insulin resistance, and increment of the pro-inflammatory cytokine TNF-α levels.

EPSI peptide single i.p. and i.n. administration 2 hours before glucose injection reduced peak glucose levels at 15 minutes in the GTT, similar to metformin treatment.

Four-week treatment with EPSI (i.n.) resulted in lower body weight in animals; the same effect was observed in mice after chronic metformin treatment. EPSI (i.n.) and Metformin-treated animals showed a 30% weight loss compared to untreated mice, while the EPSI (i.p.) administration led to a 10% weight loss. Chronic EPSI (i.n. and i.p.) and metformin treatment led to a decreased visceral fat weight, and the linear size of adipocytes compared to the HFD Control group.

At the same time, a significant decrease in feed consumption was noted only for the EPSI i.p. group on the 4th week of treatment.

A decrease in blood glucose concentration was shown for i.p. and i.n. EPSI and metformin treatments. In addition, for the EPSI i.p. group, there was also a decrease in the insulin resistance index (HOMA-IR) compared to the HFD Control group.

EPSI i.n. and metformin treatment normalized the concentration of the pro-inflammatory cytokine TNF-α to the STD control group level.

The death of 2 animals was recorded immediately after the metformin administration. Unlike Metformin, EPSI i.n. and i.p. administration caused no toxic effects throughout the entire duration of the experiment. In addition, no local irritating effect was detected after EPSI administration: the nasal mucosa after intranasal administration and the anterior abdominal wall's skin and subcutaneous fatty tissue after intraperitoneal administration were intact.

### 13.5. Illustrative Conclusion

Thus, chronic four-week EPSI peptide administration effectively alleviates symptoms of metabolic syndrome induced by the HFD in C57/BL mice similarly to Metformin, though without any adverse effects.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features. Further, the term “substantially” means that the recited characteristic, parameter, or value need not be achieved exactly, but that deviations or variations including, for example, tolerances, measurement error, measurement accuracy limitations, manufacturing tolerances and other factors known to those of skill in the art, can occur in amounts that do not preclude the effect that characteristic, parameter, or value was intended to provide. In the description presented herein, the term “about” or “approximately” refers to a range of values within plus or minus 10% of the specified number.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

What is claimed is:

1. A composition comprising a synthetic peptide, the synthetic peptide comprising or consisting of an amino acid sequence derivable or derived from one or more milk hydrolysate proteins, wherein the synthetic peptide is capable of modulating metabolism.

2. The composition of claim 1, wherein the synthetic peptide comprises or consists of about 4 to about 12 amino acids.

3. The composition of any one of claims 1-2, wherein the synthetic peptide comprises or consists of about 4 to about 8 amino acids.

4. The composition of any one of claims 1-3, wherein the synthetic peptide comprises or consists of about 12 amino acids, or about 11 amino acids, or about 10 amino acids, or about 9 amino acids, or about 8 amino acids, or about 7 amino acids, or about 6 amino acids, or about 5 amino acids, or about 4 amino acids.

5. The composition of any one of claims 1-4, wherein the synthetic peptide is defined by the general formula I:
X₁X₂X₃X₄R₁R₂R₃R₄Y₁Y₂Y₃ (I)
wherein:
X₁ is absent or a polar and negatively charged hydrophilic amino acid, optionally selected from aspartate (D) and glutamate (E);
X₂ is absent or a hydrophobic, aliphatic amino acid, optionally selected from glycine (G), alanine (A), leucine (L), isoleucine (I), methionine (M), and valine (V);
X₃ is absent or a polar and neutral charged hydrophilic amino acid, optionally selected from asparagine (N), glutamine (Q), serine (S), threonine (T), proline (P), and cysteine (C);
X₄ is absent or a polar and positively charged hydrophilic amino acid, optionally selected from arginine (R) and lysine (K);
R₁ is selected from a polar and negatively charged hydrophilic amino acid, optionally selected from aspartate (D) and glutamate (E);
R₂ is selected from a polar and neutral charged hydrophilic amino acid, optionally selected from asparagine (N), glutamine (Q), serine (S), threonine (T), proline (P), and cysteine (C);
R₃ is selected from a polar and neutral charged hydrophilic amino acid, optionally selected from asparagine (N), glutamine (Q), serine (S), threonine (T), proline (P), and cysteine (C);
R₄ is selected from a hydrophobic, aliphatic amino acid, optionally selected from glycine (G), alanine (A), leucine (L), isoleucine (I), methionine (M), and valine (V);
Y₁ is absent or a polar and neutral charged hydrophilic amino acid, optionally selected from asparagine (N), glutamine (Q), serine (S), threonine (T), proline (P), and cysteine (C);
Y₂ is absent or a polar and positively charged hydrophilic amino acid, optionally selected from arginine (R) or lysine (K); and
Y₃ is absent a polar and negatively charged hydrophilic amino acid, optionally selected from aspartate (D) and glutamate (E).

6. A composition comprising a synthetic peptide defined by the general formula I:
X₁X₂X₃X₄R₁R₂R₃R₄Y₁Y₂Y₃ (I)
wherein:
X₁ is absent or a polar and negatively charged hydrophilic amino acid, optionally selected from aspartate (D) and glutamate (E);
X₂ is absent or a hydrophobic, aliphatic amino acid, optionally selected from glycine (G), alanine (A), leucine (L), isoleucine (I), methionine (M), and valine (V);
X₃ is absent or a polar and neutral charged hydrophilic amino acid, optionally selected from asparagine (N), glutamine (Q), serine (S), threonine (T), proline (P), and cysteine (C);
X₄ is absent or a polar and positively charged hydrophilic amino acid, optionally selected from arginine (R) and lysine (K);
R₁ is selected from a polar and negatively charged hydrophilic amino acid, optionally selected from aspartate (D) and glutamate (E);
R₂ is selected from a polar and neutral charged hydrophilic amino acid, optionally selected from asparagine (N), glutamine (Q), serine (S), threonine (T), proline (P), and cysteine (C);
R₃ is selected from a polar and neutral charged hydrophilic amino acid, optionally selected from asparagine (N), glutamine (Q), serine (S), threonine (T), proline (P), and cysteine (C);
R₄ is selected from a hydrophobic, aliphatic amino acid, optionally selected from glycine (G), alanine (A), leucine (L), isoleucine (I), methionine (M), and valine (V);
Y₁ is absent or a polar and neutral charged hydrophilic amino acid, optionally selected from asparagine (N), glutamine (Q), serine (S), threonine (T), proline (P), and cysteine (C);
Y₂ is absent or a polar and positively charged hydrophilic amino acid, optionally selected from arginine (R) or lysine (K); and
Y₃ is absent a polar and negatively charged hydrophilic amino acid, optionally selected from aspartate (D) and glutamate (E).

7. The composition of any one of claims 1-6, wherein: X₁ is aspartate (D).

8. The composition of any one of claims 1-7, wherein: X₂ is leucine (L).

9. The composition of any one of claims 1-8, wherein: X₃ is serine (S).

10. The composition of any one of claims 1-9, wherein: X₄ is lysine (K).

11. The composition of any one of claims 1-10, wherein: R₁ is glutamate (E).

12. The composition of any one of claims 1-11, wherein: R₂ is proline (P).

13. The composition of any one of claims 1-12, wherein: R₃ is serine (S).

14. The composition of any one of claims 1-13, wherein: R₄ is isoleucine (I).

15. The composition of any one of claims 1-14, wherein: Y₁ is serine (S).

16. The composition of any one of claims 1-15, wherein: Y₂ is arginine (R).

17. The composition of any one of claims 1-16, wherein: Y₃ is glutamate (E).

18. The composition of any one of claims 1-17, wherein:
R₁ is glutamate (E);
R₂ is proline (P);
R₃ is serine (S); and
R₄ is isoleucine (1).

19. The composition of any one of claims 1-18, wherein:
X₃ is serine (S);
X₄ is lysine (K);
R₁ is glutamate (E);
R₂ is proline (P);
R₃ is serine (S);
R₄ is isoleucine (1); and
Y₁ is serine (S).

20. The composition of any one of claims 1-19, wherein:
X₁ is aspartate (D);
X₂ is leucine (L);
X₃ is serine (S);
X₄ is lysine (K);
R₁ is glutamate (E);
R₂ is proline (P);
R₃ is serine (S);
R₄ is isoleucine (1);
Y₁ is serine (S);
Y₂ is arginine (R); and
Y₃ is glutamate (E).

21. The composition of any one of claims 1-20, wherein the synthetic peptide consists of amino acids that do not include an aromatic, polar and positively charged hydrophilic amino acid, optionally a histidine (H).

22. The composition of any one of claims 1-21, wherein the synthetic peptide consists of amino acids that do not include a hydrophobic, aromatic amino acid, optionally selected from phenylalanine (F), tryptophan (W), and tyrosine (Y).

23. The composition of any one of claims 1-22, wherein the peptide is chemically modified.

24. The composition of claim 23, wherein the chemical modification is selected from amidation, methylation, and acetylation of one or more of X₁, X₂, X₃, X₄, R₁, R₂, R₃, R₄, Y₁, Y₂, and Y₃.

25. The composition of claim 23, wherein the chemical modification is selected from addition of formyl, pyroglutamyl (pGlu), a fatty acid, urea, carbamate, sulfonamide, alkylamine, or any combination thereof, to one or more of X₁, X₂, X₃, X₄, R₁, R₂, R₃, R₄, Y₁, Y₂, and Y₃.

26. The composition of any one of claims 23-25, wherein the chemical modification incorporates non-natural amino acids into the peptide.

27. The composition of claim 26, wherein the non-natural amino acids are selected from D-amino acids, N-methylated (or N-alkylated) amino acids, alpha-substituted alpha-amino acids, beta-substituted alpha-amino acids, beta-amino acids, and gamma-amino acids.

28. The composition of any one of claims 1-27, further comprising a pharmaceutically acceptable carrier.

29. The composition of any one of claims 1-28, further comprising a delivery vehicle.

30. The composition of claim 29, wherein the delivery vehicle is selected from a liposome, a nanoparticle, and a polysaccharide.

31. The composition of claim 30, wherein the polysaccharide is selected from cyclodextrin, chitosan, cellulose, and alginate.

32. The composition of any one of claims 1-31, wherein the composition is formulated for intranasal administration.

33. The composition of any one of claims 1-32, wherein the composition comprises at least one inhibitor of nasal mucosa proteases.

34. The composition of any one of claims 1-33, wherein the inhibitor is selected from bestatine, comostate amylase, leupeptin, aprotinin, bacitracin, amastatine, boroleucine, puromycin, a bile salt, and a fusidic acid.

35. The composition of any one of claims 1-34, wherein the composition is formulated for administration by inhalation.

36. The composition of claim 1-35, wherein the administration by inhalation is performed using a dry powder intranasal device.

37. The composition of any one of claims 1-36, wherein the composition is formulated for intravenous administration.

38. The composition of any one of claims 1-37, wherein the composition is formulated for oral administration.

39. The composition of any one of claims 1-38, wherein the composition is formulated for parenteral administration.

40. The composition of any one of claims 1-39, wherein the composition is formulated for subcutaneous administration.

41. A pharmaceutical composition comprising a therapeutically effective amount of the composition of any one of claims 1-40 and at least one pharmaceutically acceptable carrier, diluent, or excipient.

42. The composition of any one of claims 1-41, wherein the synthetic peptide is a regulatory peptide.

43. The composition of any one of claims 1-42, wherein the synthetic peptide is a biologically active peptide.

44. The composition of any one of claims 1-43, wherein the synthetic peptide is capable of modulating neuropeptide S receptor 1 (NPSR1)

45. The composition of any one of claims 1-44, wherein the synthetic peptide is an antagonist of free fatty acid receptor 2 (FFAR2).

46. The composition of any one of claims 1-45, wherein the synthetic peptide is an antagonist of G-protein-coupled receptor 43 (GPR43).

47. The composition of anyone of claims 1-46, wherein the synthetic peptide is an antagonist of G protein-coupled receptor 109A (GPR109A).

48. The composition of any one of claims 1-47, wherein the synthetic peptide is a positive allosteric modulator of lysophosphatidic acid receptor 3 (LPAR3).

49. The composition of any one of claims 1-48, wherein the synthetic peptide is an inverse agonist of muscarinic acetylcholine receptor 2 subtype (M2).

50. The composition of any one of claims 1-49, wherein the synthetic peptide induces stimulatory G protein α-subunit (Gsα)-cAMP axis in different tissues, optionally resulting in activation of intracellular Stat3 signaling.

51. The composition of any one of claims 1-50, wherein the synthetic peptide induces intracellular Stat3 signaling.

52. The composition of claim 51, wherein the synthetic peptide induces intracellular Stat3 signaling in the brain, optionally in the hypothalamus.

53. The composition of any one of claims 1-52, wherein the synthetic peptide modulates appetite regulation, glucose homeostasis, insulin resistance, and/or fat mass decrease.

54. The composition of any one of claims 1-53, wherein the synthetic peptide aregulates expression of genes involved in the pathogenesis of various metabolic conditions.

55. The composition of any one of claims 1-54, wherein the synthetic peptide activates insulin receptor substrate 2 (IRS2) gene expression.

56. The composition of any one of claims 1-55, wherein the synthetic peptide triggers a downstream anti-inflammatory effect.

57. The composition of any one of claims 1-56, wherein the synthetic peptide reduces expression levels of one or more pro-inflammatory cytokines elevated in the presence of lipopolysaccharide (LPS), optionally selected from interleukin 6 (IL-6) and tumor necrosis factor α (TNFα).

58. The composition of any one of claims 1-57, wherein the synthetic peptide lowers blood glucose and/or reduces body weight and fat mass.

59. The composition of any one of claims 1-58, wherein the synthetic peptide regulates appetite and/or an eating behavior.

60. The composition of any one of claims 1-59, wherein the synthetic peptide reduces an insulin resistance index.

61. The composition of any one of claims 1-60, wherein the synthetic peptide reduces general inflammation, optionally due to a high-fat diet.

62. The composition of any one of claims 1-61, wherein the synthetic peptide modulates insulin sensitivity, glucose tolerance, and/or inflammatory response.

63. The composition of any one of claims 1-62, wherein the synthetic peptide normalizes glucose levels in a subject with a metabolic disorder.

64. The composition of any one of claims 1-63, wherein the synthetic peptide reduces insulin concentration in a subject when administered.

65. The composition of any one of claims 1-64, wherein the synthetic peptide reduces insulin resistance index values in a subject when administered.

66. The composition of any one of claims 1-65, wherein the synthetic peptide alleviates insulin resistance conditions and/or normalizes insulin signaling when administered.

67. The composition of any one of claims 1-66, wherein the synthetic peptide reduces the body weight of a subject when administered.

68. The composition of any one of claims 1-67, wherein the synthetic peptide decreased visceral fat mass index and the linear size of adipocytes in a subject when administered.

69. A food product comprising the synthetic peptide of any one of claims 1-68, wherein the synthetic peptide is an active ingredient in the food product.

70. The food product of claim 69, wherein the food product is selected from bars, shakes, juices, yogurts, drinks, or the like.

71. The food product of claim 70, wherein the food composition includes any non-active ingredients.

72. A method for treating a related condition in a patient in need thereof, comprising administering a therapeutically effective amount of the composition of any one of claims 1-71 to a patient in need thereof.

73. The method of claim 72, wherein the condition is a metabolic disease or disorder.

74. The method of claim 72 or claim 73, wherein the condition is an NPSR1-mediated condition.

75. The method of any one of claims 72-74, wherein the condition is selected from diabetes mellitus (DM) (optionally, selected from Type 1 diabetes, Type 2 diabetes, hybrid form of diabetes (optionally, selected from immune-mediated diabetes of adults, ketosis-prone type 2 diabetes), hyperglycemia first detected during pregnancy (optionally, selected from DM in pregnancy and gestational DM)), intermediate hyperglycemia (optionally, selected from impaired fasting glucose, impaired glucose tolerance, other specified intermediate hyperglycemia, and unspecified intermediate hyperglycemia), another insulin-resistance syndrome, other specified or unspecified disorders of glucose regulation and pancreatic internal secretion, overweight (optionally, selected from overweight in infants, children or adolescents, overweight in adults, and localized adiposity), obesity (optionally, selected from obesity due to energy imbalance including but not limited by obesity in children or adolescents and obesity in adults, drug-induced obesity, obesity hypoventilation syndrome, Prader-Willi syndrome, other specified obesity, and unspecified obesity), feeding or eating disorders (optionally, selected from bulimia nervosa, binge eating disorder, and other specified feeding or eating disorders), non-alcoholic fatty liver disease (optionally, selected from non-alcoholic fatty liver disease without non-alcoholic steatohepatitis and non-alcoholic steatohepatitis), hyperlipoproteinaemia (optionally, selected from hypercholesterolaemia, hypertriglyceridaemia, mixed hyperlipidaemia, and other specified hyperlipoproteinaemia), and inborn errors of metabolism (optionally, selected from inborn errors of carbohydrate metabolism, inborn errors of lipid metabolism, and inborn errors of energy metabolism).

76. The method of any one of claims 72-75, wherein the diabetes is selected from monogenic diabetes, disease of the exocrine pancreas, endocrine disorders, drug- or chemical-induced diabetes, infection-related diabetes, uncommon specific forms of immune-mediated diabetes, and other genetic syndromes sometimes associated with diabetes.

77. The method of any one of claims 72-76, wherein the metabolic disorder is type 2 diabetes.

78. The method of any one of claims 72-77, wherein the metabolic disorder is feeding or eating disorder.

79. The method of any one of claims 72-78, wherein the metabolic disorder is intermediate hyperglycemia selected from impaired fasting glucose, impaired glucose tolerance, other specified intermediate hyperglycemia or unspecified intermediate hyperglycemia.

80. The method of any one of claims 72-79, wherein the metabolic disorder is an insulin-resistance syndrome, or other specified disorders of glucose regulation and pancreatic internal secretion, or unspecified disorders of glucose regulation and pancreatic internal secretion.

81. The method of any one of claims 72-80, wherein the metabolic disorder is overweight or obesity.

82. The method of any one of claims 72-81, wherein the metabolic disorder is feeding or eating disorder.

83. The method of any one of claims 72-82, wherein the metabolic disorder is non-alcoholic fatty liver disease optionally selected from non-alcoholic fatty liver disease without non-alcoholic steatohepatitis and non-alcoholic steatohepatitis.

84. The method of any one of claims 72-83, wherein the metabolic disorder is hyperlipoproteinaemia optionally selected from hypercholesterolaemia, hypertriglyceridaemia, mixed hyperlipidaemia and other specified hyperlipoproteinaemia.

85. The method of any one of claims 72-84, wherein the metabolic disorder is an inborn error of metabolism optionally selected from inborn errors of carbohydrate metabolism, inborn errors of lipid metabolism, inborn errors of energy metabolism.

86. The method of any one of claims 72-85, wherein the synthetic peptide is administered in combination with at least one additional therapeutic agent.

87. A method for modulating one or more of NPSR1 receptor, GPR109A (HCAR2) receptor, FFAR2 receptor, CHRM2 receptor, and LPAR3 receptor in a cell by contacting the cell with the composition of any one one of claims 1-68.
